(19) United States
(12) Patent Application Publication
(10) Pub. No.: US 2005/0085769 A1
(43) Pub. Date: Apr. 21, 2005

(54) Fluid exchange system for controlled and localized irrigation and aspiration

(75) Inventors: John M. MacMahon, Mountain View, CA (US); Thomas G. Goff, Menlo Park, CA (US); Brian K. Courtney, Toronto (CA)

Correspondence Address: ORRICK, HERRINGTON & SUTCLIFFE, LLP, 4 PARK PLAZA, SUITE 1600, IRVINE, CA, 92614-2558, US

(73) Assignee: Kerberos Proximal Solutions

(21) Appl. No.: 10/769,519

(22) Filed: Jan. 30, 2004

Related U.S. Application Data
(63) Continuation-in-part of application No. 10/198,718, filed on Jul. 17, 2002, now Pat. No. 6,827,701.
(60) Provisional application No. 60/306,315, filed on Jul. 17, 2001.

(51) Int. Cl. A61M 29/00

(52) U.S. Cl. 604/96.01

(57) ABSTRACT

The control of fluid introduction into and out of body conduits such as vessels, is of great concern in medicine. As the development of more particular treatments to vessels and organs continues it is apparent that controlled introduction and removal of fluids is necessary. Fluid delivery and removal from such sites, usually referred to as irrigation and aspiration, using fluid exchange devices that control also need to be considerate of potential volume and/or pressure in the vessel or organ are described together with catheter and lumen configurations to achieve the fluid exchange. The devices include several electrically or mechanically controlled embodiments and produce both controlled and localized flow with defined volume exchange ratios for fluid management. The applications in medicine include diagnostic, therapeutic, imaging, and uses for the introduction or removal of concentrations of emboli within body cavities.

### CROSS-REFERENCE TO OTHER APPLICATIONS

This application is a continuation-in-part of U.S. Provisional Patent Application Ser. No. 60/306,315, filed Jul. 17, 2001, and regular U.S. utility application Ser. No. 10/198,718, filed Jul. 17, 2002.

### FIELD OF THE INVENTION

The devices and related methods of the invention relate to the controlled introduction and removal of fluids in diagnostic, therapeutic and imaging applications within the body. Specifically, the invention relates to the advantageous use of a fluid exchange device in combination with a specially designed catheter to produce a system for controlled aspiration and irrigation. The systems of the invention also include fluid circuits that enhance the ability of a user to achieve selective and localized exchange of fluids within a body conduit, for example, in the diseased region of a blood vessel having a blockage or lesion. The devices of the invention, and the methods enabled by the use of the devices, have several different components that can be used individually or integrated into a system for use within an organ and within the vasculature of the body where controlled and localized irrigation and aspiration are performed together as a therapeutic or diagnostic procedure or in tandem with a separate therapeutic procedure.

### BACKGROUND OF THE INVENTION

Irrigation and aspiration are clinically important in many surgical procedures when fluids are selectively introduced into and removed from a target site within the body, usually while a surgery or other therapeutic medical procedure is performed. When the site of the therapeutic treatment is inside a body cavity or in the vasculature of the body, such as in a blood vessel, the irrigation and aspiration functions require special apparatus and methods to introduce or “irrigate” and remove or “aspirate” fluids from the target site. Surgical and percutaneous systems that both irrigate and aspirate have been developed, and some of these systems are catheter-based such that the introduction and removal of fluids is performed within an organ or a vessel by using the catheter as the conduit to introduce and remove fluids from a target site. As will be readily appreciated, the catheter allows the elements that control the fluid circuitry that directs the flow of irrigation and aspiration fluids to be remotely located, e.g., outside the body. Accordingly, the user can select from a variety of actual irrigation and aspiration functions that are provided locally within the body. Typically, the user orients the distal end of the catheter to the target site and then activates the fluid circuitry to supply and remove fluids as desired. In some cases, a medical procedure is completed simply by targeted fluid exchange, in other cases, the irrigation and aspiration functions accompany a therapeutic procedure that is performed at the target site along with the irrigation and aspiration functions.

Catheter-based irrigation and aspiration systems are unique in many respects due to their use in clinical situations where blockages or lesions exist inside a blood vessel, such as a coronary or carotid artery, and dangers arise from the creation and release of tiny particles of debris called “emboli” within the vessel. In many intravessel therapeutic procedures, the danger from the creation of emboli is an unavoidable aspect of the therapeutic procedure whenever a catheter is introduced to a target site. For example, lesions of atherosclerotic plaques inside a blood vessel are treated by several therapeutic procedures including endarterectomy, atherectomy, the placement of intravessel stents, balloon angioplasty, surgical ablation of the lesion, thrombectomy, OCT, dialysis shunt clearing and others that involve placement of a catheter near the lesion in the vessel. However, while each of these procedures offers therapeutic value in treating the lesion, each carries the risk of creating emboli during the procedure. In addition to the creation of emboli, there exists the risk of microemboli, thrombotic or otherwise in nature, which can cause substantial blockage of the microvasculature and microcirculation resulting slow flow or no reflow phenomena.

In some cases, the basic performance of the procedure inherently creates emboli, whereas in other procedures, the manipulation of the vessel and the insertion or removal of a therapeutic or diagnostic catheter is the cause of emboli generation. As with any procedure conducted in the cardiovascular system, the risk is particularly great where emboli created from plaque dislodged from inside a blood vessel travel to the brain and cause serious brain injury or death. For example, treating lesions of the carotid vessels in the neck necessarily involves high risk because any emboli that are created travel immediately to the brain. Currently, carotid treatments are attempted together with deployment of a filter or distal balloon to attempt to trap emboli generated by or released from a carotid lesion. Unfortunately, the process of moving a distal device through a clogged vessel and across a carotid lesion can generate emboli that lead to a cerebral ischemia or stroke. Schlueter et al. 2001, Circulation 104 (17) II-368. Moreover, studies have shown that crossing a carotid lesion with a structure as small as a catheter guide wire can generate emboli. Al-Mubarak et al.: Circulation 2001 October 23:104 (17): 1999-2002. Also, some lesions carry such a high risk of generating emboli that therapeutic treatments are attempted only in the most severe cases. Where a chronic total occlusion (an untreatable total blockage) exists, the diagnosis is particularly poor because it is impossible for medical personnel to place a structure beyond the point, or “distal” of the occlusion, such that emboli generated by the removal of the occlusion can be captured before entering the circulation of the bloodstream. Such chronic total occlusions can only be treated by removing the occlusion from the “proximal” side, where emboli removal is uniquely difficult. Accordingly, if the capability existed to dramatically reduce the dangers of emboli creation during therapeutic or diagnostic procedures inside a vessel or organ of the body, the existing procedures would be safer and more widely practiced, and new procedures could be performed without the problem of introducing non hazards to a medical procedure.

The generation and/or release of emboli is a concern virtually anytime a structure is passed through a susceptible vessel. Such circumstances include the placement of a balloon or stent, the placement of a filter, or simply the use of a catheter or guide wire for imaging, diagnostic, or any other procedure. In many procedures, the internal portion of a vessel is occluded to provide a segregated region of a vessel through which fluid does not flow.

For example, in the common practice of placing a stent inside an artery, a filter may be placed distally of the stent to attempt to collect emboli generated when the stent is expanded to engage plaques or lesions inside the vessel. To be effective, all such filter devices are placed distal at the treatment site and require that the filter be passed across the lesion. As noted above, virtually anytime a structure passes across, a lesion emboli of some quantity and significance are created. Thus, even when a filter is used as an added safety feature, such systems cannot protect the patient against the potential harm inherent in the placing the device itself. Additionally, once the stent is in place, the filter must be removed by pulling it through the portion of the vessel in which the stent has been inserted. This carries the risk that the filter will impact the vessel and cause the release of emboli and/or contact the stent and either displace the stent or similarly cause the release of embolic particles at the end of the procedure.

A variety of systems to contain and remove emboli have been proposed wherein a portion of a vessel that contains a lesion is segregated by two occluding members, typically two balloons, which are inflated inside the vessel at one point proximate to the lesion and at a second point and distal to the lesion. The design of these systems is to seal the inside of a region of the vessel containing a lesion prior to treatment of the lesion so that fluid exchange only occurs at the isolated region between the two occluding members. Once treatment is complete, embolic particles such as dislodged plaque are removed by applying suction between the balloons. However, the tissue of the inside walls of a vessel that is affected by a lesion is notoriously delicate and the treatment of the lesion has the capability to generate or release emboli whenever any mechanical manipulation of the portion of the vessel containing the lesion occurs.

Filters also have inherent drawbacks that cannot be completely eliminated. For example, embolic particles smaller than the filter pore size, commonly on the order of 100 microns evade filters, which must not be so small that physiologically important elements such as red and white blood cells are captured by the filter. Also, particles larger than the pore size tend to become trapped in the filter such that the filter itself becomes an occlusive element and blood flow through the filter is impeded.

Disadvantages of a two-balloon system also arise from the placement of balloons on both sides of a lesion and the nature of the blood flow that occurs in the region of the vessel containing the lesion once the balloon is removed. At the point of contact between the balloons and the vessel, plaque may be compressed underneath the balloons and may become dislodged upon reestablishment of flow through the vessel. Furthermore, many clinicians have observed that the region distal of a lesion is more likely to exhibit plaque formation than the region proximal of a lesion. This results from the disruption in the haemodynamics of the flow in the vessel due to the restriction caused by the lesion, resulting in further disease downstream. Thus, the use of any occluding member distal of a lesion does not eliminate the risk of creating emboli that may enter the vessel. The risk is particularly great when a second balloon is used because the balloon is not advantageously placed for the removal of emboli created by the use of the balloon itself and because the balloon must be removed by passing it across the lesion upon completion of a procedure. This drawback is present in all circumstances when a balloon is advanced across a lesion because, when any occluding member is placed distally of the lesion, the occluding member must be drawn back across the lesion to remove the occluding member at the end of a procedure.

Also, the placement of two balloons requires additional time to inflate the second balloon and adds to the complexity of a device due to an additional lumen that must be incorporated into the catheter to inflate the balloon. In a finite number of cases, the occluding member that is distal of a lesion, and is required to retain emboli in a defined area within the vessel, has been observed to fail, thereby releasing the emboli into the bloodstream. Because the second balloon is relied upon to prevent the flow of emboli past the region of the vessel containing the lesion, the failure of the balloon is a critical event that threatens the health of a patient undergoing the procedure. Furthermore, due to geometric constraints, the second balloon often acts as the guide wire as well. When delivering tools to perform the therapeutic or diagnostic procedure within the vessel, the balloon may move and disrupt the vessel wall or compromise the retrieval of emboli. Introduction of tools and other manipulations of a distally located balloon can also result in deflating the balloon or otherwise causing the balloon to lose patency on the interior of the vessel.

Anytime that a balloon is placed distal to a lesion, the contact between the balloon and the lesion carries the risk of damaging the vessel. For these reasons, the use of balloons inside the vessel is preferred to be minimized and the length of time and extent of contact between a balloon and the inside of a vessel should be reduced. Anytime a structure is used as an occlusive member inside a vessel, the structure must deform the vessel from the inside to create a seal about the periphery thereof with the internal surface of the vessel. For example, to make the seal tight enough to prevent the passage of fluid and emboli past the balloon, the expansion of the balloon typically deforms the vessel outward and may disrupt plaque in and about the point of contact between the vessel and the balloon. Moreover, any plaque that becomes dislodged outside the barrier formed by the balloon is released into the blood stream because there is no mechanism distal of the balloon to remove the emboli. For this reason, irrigation and aspiration proximate to the lesion are particularly important.

Ideally, the balloon or other occluding member could be placed proximal to a lesion so that the area containing the lesion would be isolated. To achieve this, the irrigation and aspiration functions would have to be provided by a structure that is positioned distal of the occluding element, such that the occluding element could be placed proximal of the lesion, and the aspiration and irrigation functions achieved distal of the occluding member.

Even under existing technologies where aspiration and irrigation are applied in a catheter based system, the parameters of fluid flow, as well as the placement of the aspiration and irrigation ports relative to an occluding member, are important to the physiological outcome for any given procedure. For example, removal of fluid and/or embolic particles by simple suction from within a body conduit may only remove a portion of the fluid present in the vessel and may leave emboli in place even if all of the fluid is removed and replaced. Deposits of plaque and other debris that may exist inside a vessel have a tendency to adhere to one another and particulate emboli tend to adhere to the sidewalls of the vessel. Thus, a system that provides limited fluid exchange is particularly unlikely to achieve a complete removal of emboli. Also, given that the interior walls of a vessel may have been contacted from within during a therapeutic procedure, a high likelihood exists that additional particles may be dislodged upon the establishment of a robust fluid flow through the vessel.

Ideally, a system for aspirating and irrigating the interior of a vessel or organ would provide both fluid exchange and fluid flow parameters that are at least similar to that experienced during ordinary physiological functions and preferably would create a turbulent fluid flow that would proactively assist in the removal of particles and other emboli. Fluid circuitry could be created outside the body that enabled high volumes of irrigation or aspiration together or independently, and either simultaneously or at selected ratios or intervals. Such a system would require both a catheter element that achieved aspiration and irrigation as well as a fluid exchange apparatus that could be coupled with the catheter to produce the desired fluid flow rates and other fluid parameters while being flexible in design and to accommodate different clinical situations and a complete range of surgical and therapeutic procedures. Because of the wide variation in intravessel procedures and the location of disease, an irrigation and aspiration system would also be particularly useful if the catheter element could be selectively positioned along a specified length of a vessel where emboli may be created together with operation of the fluid exchange apparatus to control the irrigation and aspiration flow. This capability in the catheter element is most readily created with only a single balloon system having a separate, movable, irrigation and aspiration catheter, that can move along a length of the catheter in the absence of any occluding member such as a balloon located distally of the region when fluid exchange occurs.

In the prior art two-balloon system described above, where a region of a vessel is segregated by a pair of balloons located both proximally and distally of a lesion, the area of fluid flow is limited to the region defined by the placement of the two balloons. Under these circumstances, the portions of the vessel distal of the lesion have been contacted by a balloon and are then exposed to a higher volume of fluid flow than existed before the procedure. In the context of a typical patient, a vessel which had become slowly blocked due to the deposit of plaque over a large number of years has been physically expanded by the use of an occluding member during the treatment of the lesion. Further, the therapeutic treatment at the upstream point subjects the region in which the lesion is located, and those downstream internal portions, to a fluid flow rate and volume of fluid flow that has not been experienced in the many years since the vessel began to become occluded. Under these circumstances, an even greater risk exists that plaques located downstream from the lesion will be dislodged and will enter the circulation causing serious injury.

As with ordinary irrigation and aspiration in an open surgery, the irrigation and aspiration that are applied through existing catheter systems are typically regulated only by setting the positive or negative pressure that is applied to the aspiration or irrigation lumen of the catheter and is in turn communicated to the distal end of the catheter to insert or remove fluid respectively. However, to create the specific fluid flow parameters that maximize the removal of emboli and the fluid displacement within a vessel, thereby establishing fluid change in the vessel in the most physiologically relevant manner, a specialized fluid exchange device would have to be created to regulate the fluid flow parameters of both the irrigation and aspiration functions of the system.

Accordingly, several component parts of an ideal system would be designed and implemented to maximize the therapeutic effect of the localized fluid exchange. Extracorporeal fluid circuitry components can be designed to allow rapid and volumetric fluid exchange or to allow selective irrigation or aspiration with new or recirculated fluids. At the other end of the apparatus, the distal end of the catheter can be provided with a rinse nozzle that is movable independently of the remaining structure of the catheter, and specifically, can be articulated relative to the occluding member. Moreover, the fluid parts through which the irrigation fluid is expelled into the vessel can be specifically designed to encourage fluid flow patterns that maximize the therapeutic potential of the fluid exchange process. Usually, this design encourages physiologically relevant fluid flow and flow parameters that remove loosely associated emboli from the vessel walls.

An ideal irrigation and aspiration system could be an additive component to several other apparatus that are used in therapeutic, diagnostic, or imaging applications in the body such that the capability of the system would not be exclusive of other technologies that have been applied to enhance the safety of an intravessel procedure.

Although certain portions of the discussion herein are directed towards a preferred embodiment of the apparatus of the invention used in an intravessel procedure, the devices and methodologies of the invention can readily be applied to non-vessel sites within the body such as within any body conduit such as an ear canal, colon, bowel, intestine, the trachea, lung passages, sinus cartilages, or any internal volume wherein a controlled and localized irrigation and aspiration function are desired. For example, in a diagnostic colonoscopy an endoscope may be introduced to aid in optical visualization of the site. However, the colon responds to fluid pressure changes and thus while trying to clear the field the tissue of note may move. To aid in this diagnostic situation, a controlled introduction of a clear fluid could be introduced in concert with an equivalent aspiration of dirty fluid. As such, the tissue may remain in the field of view while the process occurs. For imaging purposes the introduction of a contrast agent while simultaneously extracting an equivalent fluid will allow a vessel or organ to maintain its normal fluid level and pressure. As the imaging is completed, the same system could then return a more normal fluid to the site while extracting the foreign contrast agent. Imaging “pig-tail” catheters are presently used to introduce contrast agents to vascular system, even though radiopaque contrast agents are known to maintain a level of toxicity (Solomon, Kidney International, 1998, vol. 53, pp. 230-242). If the field of contrast was introduced and extracted as proposed by Courtney, et al., the patient's exposure would be substantially reduced.

One important medical application outside the cardiovascular system involves hollow structures in need of fluid exchange for both therapeutic and diagnostic purposes. Cysts, pseudocysts, hematomas, abscesses and effusions are variants of cavities that frequently develop within mammalian bodies and cause or are accompanied by a range of pathological conditions. All of these have different etiologies, different common locations within the anatomy, and other clinical differences, but share a generally common structure consisting of a pathologically-derived fluid or viscous material that may be contained within one or more neighboring cavities or compartments. A common feature of these structures that need medical intervention is a protected environment in which infectious pathogens can harbor and grow, can accumulate collections of toxic, pro-inflammatory and/or necrotic materials, can expand and cause mechanical interference, and can affect the proper functioning of their resident or neighboring tissues. Abscesses that rupture can lead to recurrent infections or septic shock. Cysts can rupture, leading to hemorrhage, pain or irritation. Pleural effusions can limit the ventilation capacity of lungs. Pseudocysts can rupture leading to auto-digestion of visceral organs.

A common standard of care procedure for disorders characterized by encapsulated fluid is to place a catheter or other tube within a cavity so that the fluid may be aspirated or drained. Such practice is typically carried out by interventional radiologists and other practitioners. Fluids such as antibiotics or hypertonic saline may be introduced into abscesses and other cavities suspicious for harboring infections. Similarly, thrombolytics such as tissue plasminogen activator and others may be used to help breakdown some of the fibrin-based material within hematomas and abscesses, making drainage more successful. Such fluids are typically delivered via the same catheters or tubes that are used for draining, or via a hypodermic needle. Draining catheters are often left in place for several days or weeks to allow the cavity to drain over a long period of time. However, the longer the catheters are left in place, the more time there is for an infection to occur as a result of catheter insertion. Small abscesses (e.g. <5 cm in diameter) are often simply aspirated, followed by removal of the catheters or needles used for aspiration, without allowing for any significant period of further drainage.

Pseudocysts are a special variant of abscesses that often contain enzymes produced by the pancreas and may or may not be infected. These enzymes are capable of degrading body fat and digesting proteins within the body that are necessary for normal function and structure. Pseudocysts can become very large and compartmentalized, can encroach on neighboring structures and can cause mechanical interference with proper function. Rupture of a pseudocyst is an event associated with a very high frequency of morbidity and mortality.

Accordingly, for all of the reasons described above, a novel system is needed that improves the utility of fluid exchange systems for both therapeutic and diagnostic indications, where individual parameters in fluid irrigation and aspiration and can been selectively altered and wherein the use of the system improves patient outcome in a broad range of important medical procedures.

### SUMMARY OF THE INVENTION

The present invention provides selective control of fluid exchange, including cooperative and separate irrigation and aspiration functions at a selected location within a body cavity or conduit, such as a target region of a blood vessel. The region of the body cavity which an irrigation and aspiration function are provided may include both a therapeutic treatment site, the site proximal to the placement of a balloon, or a length of a vessel both proximal to and distal of a lesion wherein a surgical treatment was performed, where a diagnostic or therapeutic procedure caused the insertion of a dye or other solution, such as a clot dissolver, or where a total chronic occlusion occurs. In one embodiment, the irrigation and aspiration functions are performed simultaneously, the fluid exchange apparatus of the invention is able to simultaneously regulate both irrigation and aspiration in a manner that advantageously controls the fluid flow rates and fluid flow parameters. This capability can be achieved both by controlling the flow rates using an electronic control system, as well as providing a mechanical apparatus that controls irrigation and aspiration flows when actuated by a user. In another embodiment, the irrigation and aspiration functions are performed separately and independently and may include traditional one-way irrigation or aspiration wherein fluid is delivered to or removed from a treatment site by direct communication through an uninterrupted fluid conduit such as an intra-catheter lumen. In another embodiment, the system may be designed to achieve internal fluid cycling wherein a turbulent flow is created at the treatment site but where net fluid exchange does not occur. In this embodiment, the fluid would be internally circulated within the fluid conduits of the invention without providing irrigation and aspiration in the conventional sense. This embodiment is particularly valuable when high value or systemically harmful pharmaceutical products are introduced to a treatment site and the circulation volume is desired to be limited for increasing concentration and reducing the amount of agent needed.

When the catheter and fluid exchange device are combined into the system of the invention, the combination provides unique capabilities for treating or diagnosing a selected location within a body conduit, particularly a pathological condition present in a body cavity or a lesion contained within a vessel. The unique capabilities are principally derived from the ability to control fluid exchange, including fluid recirculation, with turbulent flow at a treatment site while being able to selectively control the parameters of fluid exchange or fluid flow using the combined apparatus of the invention. The component parts of the invention and the design in which the components are arranged recognize that the treatment process for a pathological condition or lesion as described above is typically a multi-step process that requires special clinical, therapeutic considerations in combination with the design parameters of the medical device. For example, the treatment of a lesion in a blood vessel may involve pre-treatment prior to a therapeutic treatment, which might require ablation of a lesion or placement of a stent or expansion of the diameter of the vessel, i.e., through an angioplasty procedure, followed by or preceded by a procedure that achieves fluid exchange at the treatment site.

In a diagnostic embodiment, control of fluid exchange or circulation is important when dye or other diagnostic markers can be infused distally of an occluding member and proximate to a lesion while avoiding the potential hazards of passing a collapsed balloon across the lesion. This provides a diagnostic capability which has substantially reduced risk relative to a therapeutic treatment that requires expansion of an occluding member distal of the lesion. Moreover, the ability to selectively and independently control irrigation and aspiration functions provides the user of the system of the invention with the ability to rapidly convert from diagnostic to therapeutic indications, such as where as diagnostic dye or other marker is used to localize the placement of a catheter device within a vessel or body conduit, followed by the immediate application of a therapeutic treatment without introducing additional devices or excess fluids to the treatment site. The ability to achieve these functions while locating the catheter device of the invention, and its occlusive element, proximal of the treatment site provides an added safety margin as described above. Because of the added safety margin, both diagnostic and therapeutic procedures can be more readily performed without the risk of producing emboli and thus are more available to the clinician in treating a variety of disorders.

Preferably, the system of the invention includes a catheter element having specific features designed to facilitate the desirable fluid flow parameters when connected to a fluid exchange apparatus or to fluid conduits that control fluid exchange or fluid circulation at a treatment site. When coupled with an apparatus that provides controlled and regulated fluid flows for both aspiration and irrigation, the catheter works in tandem with the apparatus to create both controlled and localized irrigation and aspiration through a catheter-based system. For example, the apparatus of one embodiment of the invention allows the user to automatically and simultaneously control the irrigation and aspiration flow volumes, and by virtue of a specially designed catheter system, provide improved fluid flow parameters that facilitate quantitative volume exchange within a vessel or other cavity. This capability produces defined fluid flow parameters in a region bordered by an occluding element that is located proximal to a treatment site and in most cases, in a configuration absent a second more distal balloon that establishes occlusion between the treatment site and the remainder of a patient's vasculature. In this configuration, the portion of the patient's vasculature distal to the occlusion is open to the remaining circulatory system of the body to achieve the avoidance of embolism function described above. However, in this configuration, transient use of filters or other occluding elements may be used as part of a treatment procedure. Advantageously, when the second, more distal occlusion device or filter is deployed, the second device can be deployed and removed while retaining the fluid exchange and fluid circulation capabilities of the invention that can be employed to remove the embolic or other risks typically associated with the use of a second balloon or filter element.

Accordingly, the aspiration and irrigation functions provided by the fluid exchange device can be added to several existing devices such as balloon occluding elements or filters, or can be used alone as a catheter-based fluid exchange system without any additional device. Thus, the fluid exchange capabilities can be added to an existing device such as a straight catheter or filter, or an existing device can be integrated into the remaining components of the present invention to provide the advantageous irrigation and aspiration functions as described herein. For example, to decrease time during a therapeutic or diagnostic procedure, the portion of the catheter element providing the irrigation function could be combined with a catheter used to perform an angioplasty procedure.

As will be appreciated from the foregoing and following discussions, the operative irrigation and aspiration components of the invention are frequently described in the context of a catheter-based system having an occlusive element at a distal end thereof, which is often used in combination with a separate therapeutic system such as an angioplasty balloon, apparatus for placement of a stent, atherectomy, or other intravessel treatment. Furthermore, the components of the invention are frequently described in terms of the advantages derived from placement of the occlusive member at a point proximal to the treatment site while fluid exchange and replacement occurs distally of the most distal occlusive element. The integration of the irrigation and aspiration functions provides the ability to select the parameters of the fluid exchange or replacement as described here and in the examples that follow. When so integrated, the irrigation and aspiration functions are provided by irrigation and aspiration lumens that communicate fluid along the length of a catheter, irrigation and aspiration ports that are located in special configurations pursuant to this invention at the distal end of the catheter, and fluid circuitry or fluid conduits at the proximal end of the catheter that allow for selective insertion of irrigation fluids, removal of aspiration fluids, or controlled circulation of fluids within the catheter system and the treatment site accessed by the distal end of the catheter. The irrigation and aspiration lumens can be designed such that the aspiration and irrigation ports are located at any point along the catheter device, though typically at points distal to an occluding member. However, ports on opposite sides of an occluding member or other structure can be included such that a direct irrigant to aspirant volume exchange may or may not occur in the lesion of a vessel.

In preferred embodiments of the system of the invention, the catheter element provides turbulent, rather than laminar, flow within the vessel. Turbulence is introduced locally at the treatment site within the body, either through traditional fluid exchange achieved through irrigation and aspiration lumens, or through selective fluid recirculation as described below. In either case, as described below, there are several orientations for the irrigation and aspiration ports, located at the distal end of the catheter, that achieve the desired turbulent flow. Turbulent flow is specifically preferred because it reaches the walls of a body structure and facilitates both fluid exchange and dislodging of particulate matter. In a turbulent flow, the velocity at a point fluctuates at random with high frequency and mixing of the fluid is much more intense than in a laminar flow. The variations encompassed by the scope of the invention include both the placement, direction, number, and size of the ports with the ultimate goal of creating a turbulent fluid exchange within the body conduit or vessel. In one embodiment, the irrigation ports are oriented so that irrigation fluid exits the catheter element in the direction of the vessel wall. To accomplish this, the catheter element preferably has ports that facilitate fluid exit orthogonal to the wall of the distal end of the irrigation lumen of the catheter.

Also, in a turbulent flow, the velocity at a point fluctuates at random with high frequency and mixing of the fluid is much more intense than in a laminar flow. This is of particular value when attempting to clear any site of debris. Without turbulence, the flow along the sides of a vessel/lumen is approximately zero. When trying to remove/clear or exchange fluids thoroughly is it imperative to facilitate mixing. Mixing can only reach the vessel walls through the creation of fluid that affects emboli at the vessel wall. With this invention, effective, meaning therapeutically valuable, fluid turbulence can be achieved without high-powered injection systems that would carry physiological risks associated with their inherent power and abnormally high flow rates.

In more scientific terms, when a laminar flow is made turbulent, then the velocity of fluid flow will become more uniform and higher, and as a result, the vessel walls receive an improved cleansing. This turbulence is generally local to the irrigation area and controlled by the dimensions and orientation of the ports of the irrigation lumen.

The flow and velocity exchange rate through the entire system is not altered significantly because the turbulence is localized to the area around the irrigation ports. But, a turbulent flow in comparison to an equivalent laminar flow volume produces a much more uniform flow across the vessel. This results in higher velocities along the wall where emboli and thrombus are known to be in residence. From a physiological relevance standpoint, blood clots, or thrombi, are much more likely to be released into turbulent than in laminar flow. (Berne & Levy, 2001, Cardiovascular Physiology, p. 126).

Because flow is proportional to viscosity, the introduction of irrigation fluids, with any number of physiologically compatible fluid types, can increase the flow in comparison to simple aspiration of a site. For example, the viscosity of blood is 5 times that of water in a vessel larger than 0.3 mm in diameter, (from graph 5-14, in Berne and Levy, p. 129). The resulting combination of turbulence and the introduction of various fluids allows for substantially variable fluid flows which cannot be achieved without the combination herein disclosed.

Those of skill in the art will appreciate that the fluid exchange and circulation capabilities and fluid flow parameters provided by the invention can be integrated into a number of systems to provide irrigation and aspiration and essentially any physiological context where near quantitative removal of fluid or particles from a site is desired. As noted above, the enhanced fluid flow parameters can be strategically oriented relative to the placement of an occluding member, such as a balloon, to effectively remove fluids or solid matter either proximal to or distal of the occluding device. The catheter element of the apparatus can also be positioned to facilitate the removal of dyes, or therapeutic or diagnostic compounds as part of the fluid exchange function of the apparatus of the invention.

In a preferred embodiment, the invention provides both irrigation and aspiration in a selected region of a vessel proximate to a lesion, but without any occlusion distal of the lesion such that the occluding element may be both inserted and removed without passing across the lesion. As noted above, in this configuration, the vasculature distal of the occluding member of the invention is open to a patient's circulatory system and is in the absence of a more distal occluding member. In this context, it is important to appreciate that the ability to place balloons more distally of the occluding member of the invention can be provided on a temporary basis or under circumstances where a second occlusion member is placed in a separate vessel or side branch. Thus, the advantages of the invention can be provided at a local treatment site within a first vessel that may have one or more openings to the downstream patient vasculature, while a branch of the downstream vasculature is occluded. The references herein to the absence of a more distal balloon is meant to represent the absence of a more distal balloon that completely occludes the same first vessel in which the proximal occluding member is placed. Thus, one may derive the benefits of the invention by proximal placement of an occluding balloon in a first vessel, with more distal placement of a ancillary occluding member in a second vessel. This configuration provides the benefits of the invention without suffering from the recognized drawbacks of conventional two-balloon systems. Because the catheter containing the irrigation and/or aspiration components is moveable or articulatable relative to the occluding member, the introduction and removal of fluids can be achieved at several points along the vessel, either proximate to, adjacent to, or distal to a lesion within the vessel. Importantly, the point at which fluid exchange or circulation occurs is variable relative to the distal most balloon. Thus, the user of the invention can always achieve fluid exchange across a plurality of points that are distal to the most distal balloon and can ensure that emboli created distal of the most distal occluding element can be aspirated through the aspiration lumen of the catheter.

Because of the design of the catheter-based system, a single catheter element may both aspirate and irrigate and may be moved within the vessel whether or not used in combination with other apparatus. When used in combination with an occluding element, the irrigation and aspiration components may be fixed in place proximate to a lesion within a vessel or may be movable such that a single catheter element having both aspiration and irrigation functions can be advanced into an area distal of an occluding member and either proximate or distal to a lesion. When the system is actuated to perform the irrigation and aspiration function, the fluid exchange or circulation occurs both near proximate to the lesion and distal to the occlusion element. Conversely, if a more distal device is used (such as a filter or occlusion balloon), this system can be activated to accomplish the following clinical benefit. The irrigation ports being just proximal, but not exclusively proximal, to the aspiration port, the vessel can be actively irrigated with the local flow moving prograde. This drives the emboli up against the most distal occluder/filter and the aspiration port and lumen can evacuate the emboli. Thus, when used in concert with existing filters or balloons, this results in optimum retrieval of emboli from the site of active irrigation, aspiration, or fluid exchange. This embodiment does not require a proximal occlusion for clinical benefit. Additionally, this embodiment could be used independently as a therapeutic or diagnostic treatment without the addition of other interventional devices. A single catheter that both rinses and aspirates in a forward looking manner could effectively remove thrombus or other material with or without adjunctive therapies.

In procedures where emboli may be present, this device may be used as part of a method to extract the emboli generated during either a therapeutic, surgical, imaging or diagnostic procedure. The fluid volume exchange or circulation provided by the current invention is also adapted to facilitate removal of fluids within a measured portion of a vessel where vessel dimensions and fluid volumes are known. In some embodiments of the invention, the device affords a simple mechanical means through which these may occur in concert. Primary applications have been identified that produce a 1:1 exchange of fluids, but further applications include pulsatile exchange rates, ratios other than 1:1, and a closed or open loop fluid recirculation system.

The aspect of the invention that qualitatively controls fluid flow is derived in part from measured volumes that may be inserted and removed through a catheter system comprising an irrigation lumen and an aspiration lumen in fluid communication with irrigation and aspiration port(s) that insert and remove a defined or predetermined volume of solution. The design of the catheter and the fluid flow parameters achieved at the target site produce specific fluid dynamics within a vessel or body conduit that promote the removal of emboli and/or the near quantitative removal of a fluid contained in the region of a body conduit. In a preferred embodiment, a catheter coupled to a fluid exchange apparatus is actuated to create turbulence within the vessel or organ and proximate to the ports or exit holes of the irrigation lumen. As described in detail below, the size and orientation of the ports and lumen changes the fluid flow parameters such that defined flow rates, volumes, vortices, turbulence and ratios of fluids exchanged within the body can be custom designed for any application, vessel, or organ, as well as for specific diagnostic, therapeutic or imaging applications.

Because many of the embodiments of the invention are used within the cardiovascular system, the irrigation and aspiration function can be designed such that fluids move into the vasculature in a pulsatile manner as with the movement of blood within the vessel caused by the beating heart. This type of fluid movement and fluid exchange provided by the aspiration and irrigation functions of the invention is advantageous because the insertion and removal of fluid in this manner exposes the vessels or other structures to fluid flow that is physiologically relevant while a protective, emboli-remaining apparatus is still in place. Thus, the vessel experiences fluid flow that is similar to that experienced after the therapeutic, diagnostic, or imaging procedure is performed and any emboli that would be released following the procedure are more likely to be released during the irrigation or aspiration process performed by the devices of the invention. This is particularly important because the generation or release of emboli during a surgical procedure or in the immediate aftermath thereof is known to contribute to brain injury and immeasurable neurological deficit that can accompany some valuable medical procedures.

As described in more detail below, the design also facilitates a defined fluid exchange rate, such as 1:1 volume exchange that avoids damage to the vessel while producing turbulence to facilitate the removal of emboli. Generally, turbulent flows provided by the device of the invention are localized and controlled in both volume and location and are typically higher than that provided by the existing devices in terms of both flow and velocity. Target flows of 1 cc/sec are relevant to vessels such as the vein grafts, flows up to 2 cc/sec are relevant for vessels such as the carotids. (Louagie et al., 1994, Thorac Cardiovasc Surg 42(3):175-81; Ascher et al., 2002, J Vasc Surg 35(3):439-44).

As noted above, an advantage of the invention is the generation of localized turbulence in the vicinity of the infusion catheter such that volume exchange or fluid circulation within the vessel promotes the removal of debris within a vessel and the disruption of embolic particles that are only loosely attached to the interior walls of a vessel. This advantage is derived from both the design of the distal end of the catheter, including the number, orientation, and dimensions of irrigation ports, this also affects the relative location in which fluids are inserted and removed into a vessel or an organ, as well as the specific design and function of the fluid exchange apparatus that, when coupled with the catheter of the invention, combine to produce improved fluid exchange and fluid flow parameters. For example, in an ordinary vessel that is roughly cylindrical within a defined axial distance along the length of a vessel, the mere removal of liquid through simple aspiration with a conventional apparatus generally produces a laminar flow through the center of the annular structure of the vessel and the fluid along the walls of the vessel are largely left in place. With a turbulent fluid flow profile, the fluid introduced into the vessel causes an exchange between the irrigant and the existing fluid that is localized along the vessel walls, and generally causes a more thorough mixing of the fluids within the vessel such that a more complete fluid volume exchange occurs and the removal of embolic particles is enhanced.

Although the particular parameters vary according to the designs described below, the fluid exchange and fluid circulation achieved by the apparatus of the invention results in an insertion and removal of a volume of fluid from within a treatment site within a body conduit. As described in further detail below, the overall system is comprised of a fluid exchange apparatus that may have a mechanical or electrical (or both) fluid exchange component that converts a defined volume of fluid exchange with a defined axial movement of the catheter such that the volume of fluid exchanged per measure of distance of axial movement of the catheter through a vessel is known. Preferred embodiments of the fluid exchange apparatus are a substantially closed system wherein a reservoir containing irrigating fluid is combined with a reservoir containing the aspirated fluid. This invention provides several embodiments wherein known volumes are exchanged through a system that is essentially “closed” except for the exchange site within the vessel. The terms “substantially closed” mean that the system is closed because the volume of fluid inserted as irrigant solution is removed as aspirant solution in a predetermined ratio and any deviance from the ratio is attributed to only a volume of solution that is retained within the body at the target exchange site.

For example, when a system of the invention is applied to irrigate and aspirate fluid from within a vessel, the system is substantially closed because the only difference between the fluid inserted as irrigant and removed as aspirant is that which is purposefully left behind in the vessel. When the volume exchange ratio of the device is set at a 1:1 ratio, the volumetric exchange of fluids is very near to equivalent. The fluid exchange apparatus may also be actuated in such a manner that the flow produced by actuating the fluid exchange apparatus is a defined increment. Thus, a known volume of fluid is exchanged at the target site and the clinician knows with certainty the volume of irrigant fluid that is inserted as well as the volume of fluid that is aspirated out of the target site.

In one embodiment of this aspect of the invention, the fluid is recirculated within the irrigation and aspiration lumens and associated fluid conduits of the apparatus of the invention. As described in more detail below, fluid flow can be reversed in either the irrigation or aspiration lumen to provide for fluid recirculation through the target site. In this embodiment, a defined volume of fluid that is contained, in at least a portion of the catheter device, is moved in two directions within the irrigation or aspiration lumen to recirculate a defined quantity of liquid. Thus, a portion of fluid present in the irrigation lumen is introduced to the treatment site and withdrawn through the aspiration lumen, through manipulation of the fluid conduits that are external to the catheter device, the fluid flow is reversed such that the defined volume of fluid originally present in the irrigation lumen, and having passed through the treatment site and into the aspiration lumen, is reversed. This defined volume of fluid passes through the treatment site for at least a second time and may re-enter the irrigation lumen.

As noted above, and in the pertinent example that follows, this embodiment is particularly useful for high value pharmaceutical products where concentrated exposure in the therapeutic site is valuable. For example, enzymes and other therapeutic compounds that alleviate a blockage or lesion within a treatment site, such as urokinase, tissue plasminogen activators, and other such compounds, can be concentrated and continually recircled throughout a treatment site without performing quantitative volume exchange as described elsewhere herein. To enable such a system, several embodiments are possible wherein the catheter is manufactured to provide for the capability to install a closed loop to recirculate fluid. Advantageously, a simple valve system can be added to the catheter embodiment at a point external to the catheter through simple connections to the irrigation and aspiration lumen. The structural details and operation of this embodiment are described in further detail below.

In another embodiment, the device of the invention provides a 1:1 ratio of irrigation to aspiration fluid exchange such that the volume of fluid introduced to a vessel or organ is exactly matched by the volume removed. Through control of the location and movement of the device of the invention, the interior of a vessel or organ can undergo a complete fluid exchange by advancing the infusion catheter along the length of a vessel where removal of fluid is desired. By this process, several results are achieved that are beneficial therapeutically. First, as noted above, the vessel experiences a turbulence and a fluid flow that is physiologically relevant in the sense that both the volume of fluid moving across a vessel as well as the turbulence are similar to the parameters that the vessel would experience under blood pressure. This similarity has several aspects. First, the turbulence that occurs in a vessel is similar to the turbulence caused by the motion of blood moved by a beating heart. Second, the pulsatile nature of the fluid exchange is also similar to the varying pressures and pressure profile caused by ventricular contraction and the ordinary movement of blood throughout the arterial system. Finally, these specific fluid flow characteristics are achieved without producing substantially increased pressures within a vessel and without distending the vessel through the application of increased fluid pressures. Thus, the combined irrigation and aspiration of controlled volumes of liquid treat the vessel with a physiologically relevant fluid profile.

Because the device of the invention offers the ability to introduce and remove a defined volume of fluid, the clinician can have a high degree of certainty that the entire internal volume of a region of a vessel has been rinsed with an irrigation fluid by knowing the approximate internal volume of the vessel and the length of the vessel in which irrigation and aspiration are performed. This is true both for the embodiments described above wherein quantitative fluid exchange occurs in a single direction, as well as for the embodiments described wherein fluid circulation is achieved. In both cases, a known quantity of fluid exists in the system and quantitative removal of introduced fluids is possible. For example, assuming that a specified region of a vessel has an internal volume of 20 ml over a defined axial length. The device of the invention can be used to insert predetermined volumes of solution greater than, less than, or equal to 20 mls over the defined length of the vessel. Depending on the clinical environment, the ratio may be altered to remove greater volume by establishing a smaller ratio of irrigation to aspiration. One could, for example, irrigate with one volume of solution while removing twice the volume through the aspiration portion of the system to yield a 1:2 irrigation to aspiration volume.

In a preferred embodiment, the fluid exchange device has the ability to perform a controlled exchange of fluid with predetermined ratios including a 1:1 irrigation to aspiration ratio and varying ratios particularly values ranging between a 1:2 irrigation to aspiration ratio and a 2:1 irrigation to aspiration ratio. Preferably, this is achieved by having irrigant and aspirant reservoirs of defined volumes built into the fluid exchange device. However, the device can also feature a selectable control that alters the ratio of fluid exchange between a minimum and a maximum as a function of the operation of the device. In the mechanical embodiment of the fluid exchange device, each actuation of the device may cause a defined volume of fluid to be propelled through an outlet that is in fluid communication with the irrigant lumen of a catheter element. In combination, the device also features an aspirant reservoir which is expanded by a predetermined volume relative to the volume of the irrigant that is expelled.

The control of fluid exchange and fluid recirculation aspects is the result of designing the fluid flow components to cooperate with both conventional catheters as well as those specially designed to produce turbulent flow at the target fluid exchange site. The fluid control functions of the exchange device can also cooperate with the catheter element by incorporating the capability for the fluid exchange device to control motion of the catheter, specifically axial movement of the distal end of the catheter, and accordingly, axial movement of the irrigation and aspiration ports, within a body conduit such as a blood vessel. In this embodiment, the catheter element is coupled to the actuation of the fluid exchange device by a coupled translation mechanism wherein, as described in further detail below, each actuation of the device results in automatic advancement or retraction of the catheter. Thus, a defined exchange of fluid volume or a defined fluid recirculation at the target site occurs in combination with advancement or retraction of the aspiration and/or irrigation element of the catheter by a defined distance. In this manner, repeated actuation of the device provides a step-wise motion of the irrigation and evacuation functions and can insure a near quantitative volume exchange or recirculation over a defined distance. As will be apparent from the following description, this aspect of the invention provides the ability to insert, remove, or recirculate a defined volume of fluid distal of an occluding member, a capability that is enhanced with an approximate knowledge of the dimensions of the vessel. As with the other embodiments, the operation of the system may provide a pulsatile fluid flow by virtue of the application and dissipation of pressure achieved through the catheter.

Any number of designs for the fluid exchange apparatus can be used to provide controlled volumes of irrigation and aspiration fluid, through the catheter element of the invention to the target exchange site. The simplest embodiment of the invention provides a squeeze bulb wherein the irrigant and aspirant reservoirs are typically separated by a membrane and are in fluid communication with a irrigation and aspiration lumen that communicate fluids to and from the target site. In this embodiment, a one-way valve is provided preferably on both the irrigant and aspirant side of the fluid flow, to prevent aspirated fluid from flowing back to the target site. In another embodiment, a mechanical device causes pressure to be exerted on an irrigant reservoir that is in fluid communication with an irrigation lumen that provides fluid flow to at least one irrigation port at the distal end of a catheter. The catheter element also comprises an aspiration lumen, that may or may not be integral with the irrigation lumen, and which facilitates fluid communication of the aspirant fluid back to an aspirant reservoir. In this embodiment, the irrigant is expelled from a reservoir by the application of mechanical force to reduce the volume of the irrigation reservoir and the mechanical force is preferably coupled to an expansion of the volume of the aspirant reservoir to yield a defined fluid exchange between the irrigant reservoir and the aspirant reservoir.

In one preferred embodiment, a hand-held mechanical device is actuated by a trigger to insert and remove controlled volumes of fluid through the catheter element. The hand-held embodiment is comprised of an actuator such as a movable trigger that is mechanically operated by being grasped by the hand and pulled towards a stationary structural housing of a complementary portion of a housing to cause a reduction in the volume of an irrigant reservoir and, accordingly, fluid movement through an irrigation lumen and out one or more irrigation ports at the distal end of a catheter. Fluid provided to the target site in this manner is recovered through one or more aspiration ports and communicated through an aspiration lumen and returned to the aspirant reservoir of the fluid exchange device. The irrigant and/or aspirant fluids are preferably contained in a sealed reservoir system such as a cylindrical chamber having a piston and a rod wherein the piston is mechanically coupled to the actuating element. Motion of the actuating element transfers force to the piston and causes contraction of the irrigant reservoir and expulsion of liquid from the reservoir. Simultaneously, the motion of the actuator causes the expansion of the volume of the aspirant reservoir and causes withdrawal of fluid through the aspiration lumen and into an aspirant reservoir. In such an embodiment, the actuation of the trigger may translate into varying amounts of fluid flow depending on the mechanical expedients used. A single actuation of the trigger may translate into an incremental movement of a piston that exerts force on an irrigant and/or aspirant reservoir.

By the use of several conventional mechanical apparatus, such as a ratchet and gear mechanism, a lever and pivot system, or others, the mechanical fluid exchange device exerts a direct control over the exchange of fluid communicated through the irrigation and aspiration lumens. The control of the fluid and the particular features can be provided in several designs that achieve the same function. For example, in addition to the hand-held apparatus described below, the force needed to create the fluid flow in both the aspiration and irrigation sides of the system could be provided by a mechanical foot pump, vacuum pump or virtually any component device that provides controllable fluid flow. Moreover, to provide total reproducibility in the operation of the system, a console controlled by a computer with appropriate commands or a software program is readily used to produce the same fluid flows, fluid exchange parameters, including exchange ratios, and essentially all of the functions of the purely mechanical embodiments described below. Therefore, those of ordinary skill in the art will appreciate that any number of mechanical or electrical variations give rise to the same fundamental principle wherein controlled volumes are applied to a target site through a segregated irrigation and aspiration system, preferably comprised of irrigation and aspiration lumens that pass through at least one catheter element and engage in fluid exchange at a target exchange site by virtue of specially designed irrigation and aspiration ports at the distal end of the catheter element.

By altering the dimensions of the irrigation reservoir and the aspiration reservoir, the ratio of fluid exchange between the irrigant and aspirant reservoirs is altered and, accordingly, the fluid exchange in the target vessel is adjusted. For example, where the irrigant reservoir and aspirant reservoir are of identical sizes, an actuation of the fluid exchange device may yield a 1:1 fluid exchange within the target vessel. Where, as described above, a different fluid exchange ratio is desired, the difference in the ratio may be achieved by a corresponding difference in the dimensions of the irrigant and aspirant reservoirs that are emptied and filled through the operation of the fluid exchange device. Also, variations in ratio may be accomplished by corresponding changes in the dimensions of in-line chambers as described below. Likewise, with a 1:1 ratio, equal volumes of irrigant and aspirant are exchanged in a single cycle of the fluid exchange apparatus. In the 1:1 embodiment, the entire irrigation and aspiration volumes may be exchanged within a defined number of cycles of the apparatus. For example, one may provide that each cycle of the hand-held apparatus provides 1 ml of irrigant volume and removes 1 ml of aspirant volume. By providing an irrigation and aspiration reservoir with known volumes, a known number of cycles translates into a known volume of irrigation and aspiration.

As noted above, in one specific embodiment, the actuation of the device also causes translation of the infusion catheter along a defined axial path such that a known volume of solution is provided in both the irrigation and aspiration aspects as a function of the distance that is traveled by the infusion catheter. As noted above, in some clinical situations, turbulent flow is desired without complete fluid replacement such that fluids are desired to be recirculated through the treatment site. In these situations, it is desirable to cycle fluid back and forth near the distal end catheter using the infusion and aspiration lumens and ports without causing a new replacement of fluid. An example of such a situation would be in the use of therapeutic thrombolytics, where the benefits of turbulent flow in combination with the enzymatic action of the agent break down a clot. In addition to cost, many such agents depend on a blood component, such as plasminogen, to be effective. Also, if a volumetric fluid exchange resulted in removal of all the blood in the region to be treated, no remaining blood component, such as plasminogen, would be left to be activated by the drug. Furthermore, temporarily introducing an agent in an irrigation fluid and then removing it quickly through aspiration may unnecessarily remove active agent and add to the cost of treatment by requiring a higher amount of the drug to be used.

A simple adaptation of the system of the invention enables a closed recirculation system having modes of operation. The first mode incorporates the uni-directional flow patterns described herein, where an infusion lumen infuses fluid and an aspiration lumen removes the liquid through direct aspiration. The second mode of operation effectively disables the function of the one-way valves and cause a different flow sequence as a result. In this mode, the flow in each of the lumens would be bidirectional. For example, the infusion lumen continues to infuse fluid into the treatment site and the aspiration lumen continues to aspirate fluid from the treatment site. (Alternatively, the designated “infusion” lumen could aspirate first and the designated “aspiration” lumen could infuse first). However, the aspiration lumen would re-infuse the fluid just aspirated, and the infusion lumen would aspirate fluid from the treatment site, a portion of which would likely be fluid that had just been infused. The net effect of this second mode of operation would be to cause turbulent pulsatile flow in the region proximate to the distal end of the lumens without causing a net replacement of the fluid in that region.

Clearly, the irrigation reservoir may advantageously be divided into subparts and is not limited to ordinary aqueous solutions used in a surgical context. Given the utility of the present device for diagnostic and imaging applications, the irrigation reservoir could be filled with dyes, contrast agents, or other solutions that aid in the diagnosis or treatment of the vessel. Given that the fluid exchange device of the invention also provides unique fluid flow parameters, the irrigation reservoir could contain any therapeutically valuable solutions such as heparinized ringers lactate, antibiotics, anti-angiogenics, anti-neoplastics, or any other thrombus or emboli treatment fluids that are used to perform the therapeutic procedure on the internal portion of a vessel or organ. Given the ability to specifically tailor the fluid exchange and fluid circulation parameters for a target vessel, the device offers the ability to use therapeutic compounds that might not otherwise be available because the clinician can be certain of the enhanced ability to remove solutions introduced via the irrigation reservoir. The fluid exchange apparatus can also be used to promote absorption of a therapeutic layer on a vessel wall. If a drug coated stent is produced that can reabsorb drugs after they have eluted, then with this device a high concentration of the drug can be introduced and pooled about the stent for a brief period. This high dose may then be absorbed or bonded back to the structure or one of its components and thereby recharging the drug coated stent.

In a system where it may be advantageous to have ratios other than 1:1 in the system it is also directly applicable. For example, in another vascular situation a virtual shunt may be created where a proximal fluid can be circulating and a fluid is infused distally. This would involve a ratio of greater than 1:1 irrigation to aspiration. Furthermore such an arrangement could introduce a second fluid to be the primarily distally delivered fluid. The second fluid could be blood, blood substitute, plasma or oxygenated fluid to produce a virtual shunt.

In the diagnostic use of optical coherence tomography, OCT, the fields of applications are presently limited by the need for a clear field. Similarly the use of intravascular ultrasound, IVUS, is somewhat limited by the attenuation associated with the blood in vivo. A substantial volume exchange of the vessel region in proximity of the distal end of the OCT or IVUS catheter would provide the opportunity to replace blood or other fluids with transparencies other than that found in blood, thus improving and/or modifying the imaging quality. In applications outside the cardiovascular area, a significant advancement in the standard of care for many fluid-filled cavities, such as those described above, would be to replace the drainage catheter with a catheter element of the present invention that provides multiple lumens to enable the simultaneous infusion and aspiration of fluids to achieve fluid exchange at the treatment site. Such a system would enable the replacement of potentially harmful contents of the cavity with more physiologically and pathologically inert material, such as saline. This replacement or exchange of fluids could also be an adjunct to the normal draining that is the current standard practice. The draining could occur through any of the one or more of the lumens described for infusion or aspiration on a combination thermal and preferably accompanies use of the fluid exchange apparatus described herein.

There are several potential advantages of replacing fluids within cavities either in place of, or in combination with subsequent aspiration or drainage. By introducing a less viscous fluid into the cavity by irrigation, any subsequent drainage could occur more quickly, and some of the fluid may be reabsorbed. By substantially removing and replacing infectious pathogens and their products (e.g. products of degradation or secreted toxins) with sterile fluids, or fluids of a lower infectious potential, the likelihood of complications secondary to infection may decrease substantially. Such complications include, but are not limited to, dissemination of infection to other tissue sites, septic shock, and disseminated intravascular coagulation, the latter two of which are associated with extremely high rates of morbidity and mortality. In the particular case of pseudocysts, the highly dangerous auto-digestive enzymes would also be removed or substantially diluted via a fluid replacement system.

In some digital situations, delivery of a therapeutic fluid to such cavities during the process of exchanging the fluid is indicated. One important indication requires the delivery of antibiotic agents to abscesses and other potentially infected cavities. Antibiotics are currently introduced using a single catheter system, such as through a drainage catheter, but not in a system that incorporates the concept of substantial fluid exchange or replacement. Some antibiotics, such as aminoglycosides, have their efficacy determined by their peak concentration rather than their average concentration over time. By introducing such an agent in high concentration to a localized region for a short period of time, and then removing or replacing a substantial portion of the fluid, a highly therapeutic effect can be achieved, with minimal side effects such as nephrotoxicity as in the case of aminoglycosides. Fluids containing materials toxic to pathogens are also introduced pursuant to this invention, such as hypertonic or hypotonic saline, alcohols, antiseptics and others. The ability to locally deliver and to subsequently remove such substances from the cavity, which may be toxic to the patient if they were to disperse elsewhere in the body, increases the range of fluids which could be used to treat such localized pathogens.

Moreover, chronic pleural effusions, abscesses, pseudocysts and hematomas can develop loculations (localized regions that are partially or completely walled-off from the rest of the cavity). These loculations are thought to result initially from fibrin cross-linking, followed by scar-tissue development and are sometimes treated with small caliber catheters to deliver fibrinolytic agents such as tissue plasminogen-activator, streptokinase or urokinase, followed by traditional drainage and/or aspiration. The combination of irrigating and aspirating such agents, or introducing such agents followed by simultaneous irrigation and aspiration a short time thereafter may accelerate the treatment of these effusions. The aforementioned fibrinolytic agents are often referred to as indirect fibrinolytics, as they activate native plasminogen which must be present in the region in order to produce plasmin which degrades fibrin. The current invention may also provide similar or enhanced efficacy if used with direct-acting fibrinolytics, such as Alfimeprase and other enzymes similar in action to fibrolase. These direct-acting fibrinolytics, originally extracted from the venom of certain species of snakes, are not dependent on the presence of native blood components, such as plasminogen, and are capable of directly cleaving fibrin.

Regardless of the specific fibrinolytic agent, the ability to introduce fibrinolytic agents and then safely remove them using a fluid-replacement system would provide significant advantages by speeding up the drainage of these cavities as the degree of loculation would be reduced. An acidic or alkaline solution may also be useful in breaking down these loculated buildups within the cavities, as may a solution which is heated above normal body temperature. By delivering these agents locally, and having the ability to remove these agents via simultaneous irrigation and aspiration with another fluid, such as saline, the systemic and/or toxic effects of these therapeutic agents can be substantially minimized and higher concentrations of these agents may be used locally for greater efficacy of action.

Other possible agents that could be introduced and then replaced for this specific therapeutic purpose and others described herein include radioactive components, cytotoxic agents, alcohol solutions or other materials and formulations that have the potential to alter the biological function of the cells that reside near the surface of a cavity's walls. Pseudocysts, cysts and effusions are often lined by secretory cells that cause the accumulation of secreted fluids within the cavity. The induction of an inflammatory and/or fibrotic reaction via an irritant such as an alcohol, or the induction of cell death or a change in cell function for these secretory cells would provide benefit by reducing the likelihood of recurrence of fluid accumulation within the cavity.

As noted above, these different types of cavities can often be compartmentalized, necessitating the insertion of several drainage catheters to be able to drain each of the compartments to achieve the desired effect. Due to imaging limitations, it is often not known how many compartments within the cavity exist, or what their boundaries are. In conventional treatments, catheters are inserted in a few locations based on a best approximation of the compartment boundaries seen on imaging. However, several days may be needed before the medical practitioner can detect that not all compartments of the fluid collection have been catheterized to allow for sufficient drainage. This delay in complete treatment often complicates the course of disease and extends the overall length of time for effective treatment. With a fluid replacement system, it will be possible to better visualize the boundaries of these compartments. One method for such an improved visualization could occur by substantially replacing the contents of a cavity compartment with a radio-opaque solution via simultaneous irrigation and aspiration. The region of the cavity can then be imaged using radiographic techniques, such as CT, to see if the compartment that has been accessed via the catheters is representative of the entire region of pathological interest, or if there are other compartments within the same pseudocyst or other such cavity that require further treatment. Other imaging modalities may also be used with appropriate contrast agents being introduced into the cavity, such as gadolinium for magnetic resonance imaging, microbubbles or echolucent fluid (such as saline) for ultrasound imaging, and radioactive isomers for nuclear medicine scanning. Alternatively, the use of a fluid that is substantially translucent in the wavelengths of interest (e.g. visible spectrum for visible light, infrared spectrum for infrared light) could facilitate direct visualization within the cavity by delivering a fiber optic or imaging detector (such as a CCD camera) into the cavity via one of the lumens provided by the system.

The pattern of the irrigation parts near the distal end of, and in fluid communication with, the infusion lumen(s) can be such that the flow pattern resulting from the irrigation is either focal, or diffuse. A focal infusion can be used to induce a relatively simple flow pattern between the point of infusion and the point of aspiration. At the other extreme, a diffuse, multi-port spray pattern can more globally perturb the contents of the cavity which may cause material clinging to the walls of the cavity to be released such that it can be aspirated.

A 1:1 ratio between irrigation and aspiration is often desired because no net effect is made on the size of the cavity and 1:1 may be the ideal ratio for general fluid replacement. However, it may be desirable to transiently irrigate use more fluid than is aspirated in order to encourage the cavity to expand. This could be of use in those cases where the clinician deems it desirable to temporarily mechanically expand the cavity. By temporarily increasing the volume of the cavity, some regions of the cavity that might not be fluidly communicating with the region of the aspiration catheter, either due to collapsing of the walls or scar tissue holding opposing surface together, may be made to enter fluid communication with the fluid replacement system. On the other extreme, it is an important goal of many of these procedures to reduce the volume of cavity contents and a ratio of less than 1:1, such as 0.5:1, would be desirable in those instances.

With respect to methods of use for the fluid replacement system, a combination of traditional draining and a series of 1:1 fluid replacements may be used to provide the benefits described above. A typical sequence would be to introduce one or more draining catheters into the cavities of concern and allow for some initial draining as the contents of the cavity may be under increased pressure relative to their surrounding environment. Subsequently or simultaneously therewith fluid exchange is used, typically in a 1:1 ratio, although other ratios may be desirable under different circumstances. By way of example, an initial attempt to rid the cavity of its potentially dangerous contents could be done using saline as the replacement fluid of choice. Optionally, the saline may include some imaging contrast agent to assist in visualizing the efficacy of fluid replacement by fluoroscopic or other means. The operator may elect to use several times more fluid to rinse the cavity than the cavity actually contains so that a more effective rinsing and dilution of the pathological contents can take place. The simultaneous irrigation and aspiration of fluid makes this possible in a very convenient manner. Once a substantial portion of the native contents of the cavity have been removed, the operator may then use a fluid that contains some therapeutic or diagnostic purpose to replace the fluid that was used for initial rinsing of the cavity's contents. This use may be repeated several times over with combinations of different agents for the desired therapeutic or diagnostic effect and the periods of fluid replacement may be separate by periods of time to allow these agents to take effect. Then, the therapeutic and diagnostic fluids could optionally be replaced by saline or some other substantially physiologically inert fluid such as saline. One or more of the catheters introduced may be removed. One or more may be left to allow for continued draining as per the traditional therapeutic regimen of draining, and/or to facilitate access for further fluid replacement treatment in the near future.

The benefits of such a system would include improved therapeutic outcomes, reduced hospitizations and repeat procedures, and reduced time of treatment, all of which have substantial significance in the well-being of patients as well as for the efficient and economic delivery of health care. The broad applicability of a system that provides for fluid replacement within these cavities suggests that the system is highly generalizable, and the aforementioned set of uses, agents and fluids that could be delivered via such a system are non-limiting examples of the scope of range of uses.

One of the important aspects of the infusion portion of the fluid replacement system is that the irrigation can consist of a locally turbulent flow that can increase the concentration gradient of active variants of a drug near a surface. Furthermore, such irrigation can provide mechanical impetus for the breakdown of the components that are to be removed and can increase the likelihood of releasing their attachment to the walls of the cavity. Several different optimizations in shape and profile of the irrigation catheters as described herein could be envisioned to assist in the flow patterns produced to optimize their benefit within such cavities. These catheters may also be designed to be translatable relative to an occlusive member and are rotatable to allow for some directional control of the irrigant flow that is released.

Those catheters whose outer walls are in direct contact with the wall of the cavity at the point through which the cavity was entered via interventional means may incorporate expanding members on their outer surface to prevent them from slipping out of the cavity prematurely, and to improve the seal at the site of entering the cavity in order to reduce the likelihood of noxious substances from escaping the cavity by means other than the lumens of the catheters. Such expandable members can include one or more balloons, or structures made of open-cell foam that is self-expanding. Optionally, there may be a soft, pliable sheet of material attached to the outer wall of the catheter that can deployed within the cavity. This sheet could act like an apron that helps seal the site of entry into the cavity.

Those skilled in the art of medical devices will appreciate that all of the component parts of the invention are assembled from biocompatible materials, typically medical plastics or stainless steel. The syringes described below may be ordinary medical-use syringes or may be custom fitted to be replaceable and to fit engagingly with the fluid exchange apparatus. An irrigant reservoir that is integral with the device may be pre-filled or a pre-filled syringe may be used to supply the irrigant fluid. In either a stainless steel or plastic embodiment, the device is sterilized. Typically, stainless steel devices are exposed to heat and steam in an autoclave, while medical plastics may be exposed to gamma irradiation or microbicidal gases such as EtO. The methods of the invention specifically include the use of any component of the system of the invention followed by sterilization of the components, or the entire system, and re-packaging for subsequent use. Although plastic embodiments are designed for single use, sterilization may be performed to functionally reconstruct the utility of the device after use with a patient.

### DESCRIPTION OF THE DRAWINGS

FIG. 1 shows the basic components of the device necessary for implementation with the optional inclusion of components that generate a minimum flow rate of exchange, components that incorporate an upper flow rate of exchange, and a configuration where a combination of flow threshold and ceiling provide a flow rate bandwidth.

FIGS. 2A-2D are cross-sections of a vessel showing the catheter element of the invention with aspiration and irrigation lumens combined in the same catheter element and terminating at an aspiration and irrigation port, respectively. FIG. 2A is a section of the catheter showing the aspiration and irrigation lumens. FIG. 2B is insertion of the catheter element into an exchange region established at a terminal lumen characterized by a total occlusion such as a clot, lesion, abscess, a ball of wax or a body conduit or organ that is closed-ended such as an ear canal. FIG. 2C shows a cross-section of the system with an occlusion balloon to establish a defined region of fluid exchange between the irrigation lumen and the aspiration lumen. FIG. 2D shows one example of the placement of an aspiration port and an irrigation port that is in fluid communication with the aspiration lumen and irrigation lumen, respectively.

FIGS. 3A-3G show the catheter element in various configurations and illustrate the difference between laminar and turbulent flow. FIG. 3A is a catheter element having an occlusion member and comprising an occluding guiding catheter having an aspiration lumen and with the irrigation provided by a separate catheter to aid in defining a field of exchange. FIG. 3B shows a catheter element providing an isolated, localized region for fluid exchange that is maintained by irrigation occurring both proximal and distal to a centrally disposed aspiration port. FIG. 3C shows a typical laminar flow that fluids will naturally assume when passing through a cylindrical tube. The flow velocities are highest at the center of the tube and approach zero velocity at the walls of the tube. The length of the arrows indicate the magnitude of the velocity.

FIG. 3D shows the turbulent region of flow created by a catheter element of the invention adjacent to a region where the flow transitions to a laminar flow, but still has a comparatively higher velocity along the walls of the tube. At a distance from the irrigation ports, the flow achieves laminar flow.

FIG. 3E shows a catheter element with 3 rows of perfusion holes. The figure illustrates how the turbulent flow is most pronounced in the immediate vicinity of the infusion ports and begins to assume laminar characteristics until the next row of infusion ports is encountered. In the region designated “A,” turbulent flow is provided by the irrigation port geometry. In region “B,” flow is tending toward laminar flow. In region “C,” laminar flow is established.

In FIG. 3F, the various regions of flow show the relative distances necessary for each activity. The transition region has typically been shown to be about the same length as the perforated region of the catheter element. In FIG. 3G, a two-catheter system for fluid exchange is provided without an occlusive member at the distal end of either catheter. The two catheters are concentrically oriented and slidable relative to one another.

FIG. 4A is a schematic of an embodiment of the fluid exchange device that produces pulsatile flow through the application of leverage to a hand-held unit that is actuated to communicate force to the irrigant reservoir and which collects fluid in the aspirant reservoir. FIG. 4B is an embodiment that accepts interchangeable fluid cartridges, similar to syringes, for irrigation and aspiration and where the exchange rates can be altered to other than a 1:1 ratio. In this example there is a 2:1 ratio of irrigant to aspirant dictated by the relative sizes of the fluid cartridges. FIG. 4C is an embodiment to create a layered approach to aspiration (or irrigation) wherein an additional fluid compound is attached to a lumen to provide a manually adjustable added fluid flow.

FIG. 5A is a fluid exchange device incorporating a segregate irrigant reservoir that uses different types of irrigants, while FIG. 5B segregates the irrigant fluid into a sample to be inserted both proximal to and distal at a point of the target site.

FIG. 6 is a tabletop version of the fluid exchange device that is suitable for either a mechanically drive hand system or an electronically controlled, pump-driven system, including an optional in-line air trap for the irrigant and a filter for the aspirant.

FIGS. 7A and 7B are a grip lever activated embodiment of the hand-held fluid exchange device of the invention wherein the actuation of a trigger relative to the body of the handle translates into the motion of a piston that propels fluid from the irrigant chamber and collects fluid in an aspiration chamber (not shown).

FIG. 8 is a preferred embodiment of the hand-held fluid exchange apparatus of the invention having a spring tensioned trigger mechanism that is actuated by manual motion of the trigger relative to the body of a handle. Actuation causes linear or incremental motion of a dedicated irrigant and aspirant carriage that move in opposite directions to control the force supplied to the irrigant and aspirant reservoir, respectively.

FIGS. 9A and 9B illustrate an embodiment at the hand-held fluid exchange device having an adjustable pivot point on a trigger to produce different flow rates and peak pressures.

FIG. 10 is an embodiment wherein the control of the movement of pistons that propel fluid from a cylindrical irrigant reservoir and into an aspirant reservoir is provided by a ratchet mechanism.

FIG. 11 is a fluid exchange device with two chambers, such that both an irrigation and aspiration chamber are arranged to operate in concert, with one filling and one expelling fluid in each direction and having separate input and output pathways for connecting to the reservoir and lumen elements.

FIGS. 12A and 12B show the apparatus configured as a compressible ball squeezed by the hand with the internal volume divided into irrigant and aspirant chambers and designed to be connected in-line with irrigation and aspiration lumens and reservoirs.

FIGS. 13A and 13B are an embodiment wherein the fluid exchange device is a hand ball pump configured with an internal reservoir of irrigant fluid and a flexible member to separate the irrigant from in-flowing aspirant fluid. This device is initially loaded with a volume of irrigant that encompasses most of the initial internal volume of the ball and which flows through the target site to the internal aspirant reservoir. FIG. 13C is an embodiment having a substantially rigid external housing and an internal balloon. The interior of the housing is filled with fluid and an internal balloon containing air or a non-volatile gas. A volumetric pump changes the internal configuration of the balloon to force fluid from an internal irrigant reservoir to an internal aspirant reservoir.

FIG. 14 is a device with both irrigant and aspirant chambers combined into one housing separated by a movable piston into two distinct chambers to allow for the simultaneous rinsing and aspirating.

FIG. 15 shows a slidable and threaded combination configuration where an irrigant can be driven out and an aspirant simultaneously drawn in by both a sliding and a screw-type mechanism. The sliding provides gross travel and the rotation of the member about the axis produces a fine-tuning mechanism.

FIG. 16 is an embodiment of the fluid exchange device that can be comprised of as few as the structural elements that preferably attach to a cylinder body of one reservoir and piston of the other.

FIGS. 17A and 17B are a mechanical fixture for providing a self-advancing or retractors catheter element in combination with the fluid exchange device.

FIGS. 18A-18C are an embodiment of the invention with a staging capability such that the means for aspiration and irrigation are linked mechanically to travel in equivalent and opposite directions.

FIGS. 18D and 18E are embodiments of the invention with an isolated irrigation or aspiration function provided by a single fluid compartment device that may be actuated by hand. Advantageously, a stepwise increase in pressure can be provided using a one-way valve.

FIGS. 19A through 19F are embodiments of the invention providing a recirculation loop to control and repeatedly cycle fluid through a treatment site.

FIG. 20A is an embodiment of the distal end of the catheter element of the invention having an aspiration port distal to irrigation ports.

FIG. 20B shows an embodiment with the irrigation ports 6 and aspiration ports 9 strategically arranged for use with the selective “fluid exchange” or “recirculation” embodiments. In this embodiment, the ports 6, 9 are larger and geometrically arranged to allow for greater exchange of fluids and material in the recirculation mode. Additionally, this arrangement has forward (distally) oriented ports 6 which aid in the delivery of fluids and agents ahead (distal) of the catheter 7.

FIG. 21A shows an arrangement of ports which increase slightly in size approaching the proximal end of the catheter. The inverse, with the holes increasing in size toward the distal end, is also possible.

FIG. 21B shows an arrangement of ports in which the ports have an increased density toward one end of the catheter.

FIG. 22 shows a series of uniform ports along the distal end of the catheter. The length of the region of introduction ports could be varied to achieve the desired clinical result.

FIG. 23A shows another arrangement of ports on the rinse tip to provide a longitudinally expanded area of rinse.

FIG. 23B shows a multiplicity of ports in the rinse tip to provide expanded coverage of the rinse and increased diffusion of the force and/or velocity of the fluid that is to be ejected.

FIG. 24A is a perspective drawing of another rinse tip configuration that provides for the redirection of flow proximally.

FIG. 24B is a side view of a rinse head showing one manner in which the redirection of the fluid could be achieved.

FIGS. 25A and 25B show another configuration for a rinse head. FIG. 25A shows the tip in the unexpanded configuration for a minimal profile for insertion and/or removal. FIG. 25B shows the tip in its expanded form, such expansion provided by the forceful ejection of fluid through the holes located to one half of the balloon. This allows for flow of the rinsing agent in the proximal desired direction.

FIGS. 26A and 26B show another configuration for a rinse head to achieve proximal flow of fluid. FIG. 26A shows the tip in its unexpanded form for minimal profile for insertion and removal. FIG. 26B shows the expanded structure which creates a flow in the proximal direction as a result of its geometric arrangement.

FIGS. 27A and 27B show a possible configuration of a rinse tip that would provide directional flow. FIG. 27A shows the tip in the passive state with a low profile for insertion and removal. FIG. 27B shows the tip in the active state, with the flap pushed out by the ejecting fluid.

FIGS. 28A, 28B, 28C show a few construction techniques which could be used to achieve a design with an over flap. FIG. 28A shows a band that circumferentially secures the flap to the rinse tip. FIG. 28B shows the flap secured by heat bond or adhesive. FIG. 28C shows the flap as a co-molded piece with the rinse tip.

FIG. 29 shows a construction technique where the rinse flap is inset so that it is either flush with the edge profile of the catheter, or alternatively, it could be recessed.

FIGS. 30A and 30B show a manifestation where the flap is pleated to allow for expansion and reduction. The flap could also be made of an elastic material that expanded under pressure, or a non-conforming material with some plasticity. FIG. 30A shows the undeployed, low profile situation. FIG. 30B shows the device with the flap in the expanded configuration.

FIGS. 31A and 31B show a configuration of the flap containing support ribs connected by webbing. FIG. 31A shows the undeployed configuration for insertion and removal. FIG. 31B shows the expanded flap supported by the ribs.

FIGS. 32A and 32B are longitudinal cross sections showing a tip of the fluid introduction catheter where the flap covers only a portion of the ejection ports. FIG. 32A shows the tip with the flap in the default, collapsed position. FIG. 32B shows the tip with the flap extended by the ejecting fluid.

FIGS. 33A and 33B are longitudinal cross sections showing the distal rinse tip catheter with a flap of variable thickness. Varying the thickness of the flap allows for the achievement of desired response in flexibility of the flap, and allows for a thin, soft outermost edge of the flap. FIG. 33A shows the tip with the flap in the default, collapsed position offering a low profile for insertion and removal. FIG. 33B shows the tip with the flap in the extended arrangement with fluid being expelled by the catheter.

FIGS. 34A and 34B are cross sectional drawings showing another configuration of the rinse catheter where each port, or ring of ports, has its own dedicated cover flap. FIG. 34A shows the flaps in the default, collapsed setting offering a low profile for insertion and removal. FIG. 34B shows the flaps in the expanded configuration with fluid being ejected through the ejection ports.

FIGS. 35A and 35B show side views of the catheter tip illustrating the flaps that cover the ports. FIG. 35A shows the flaps in the collapsed position for insertion or removal. FIG. 35B shows the flaps expanded, allowing fluid to be ejected through the ports.

FIG. 36 is a cross-sectional side view of the distal tip of the catheter. The arrows show a slight suction applied to the interior lumen of the catheter to hold the flaps in close to the catheter. This technique secures the flap(s) to the catheter for a low profile upon insertion and removal.

FIGS. 37A and 37B show another embodiment of an expandable ejection membrane for the introduction of fluid into the vessel. FIG. 37A shows the device in its collapsed, low profile form for insertion and removal. FIG. 37B shows the device expanded and delivering fluid to the vessel.

FIGS. 38A and 38B show the distal tip of a fluid introduction catheter utilizing cover flaps to direct the flow of the fluid being injected. FIG. 38A shows cover flaps open under the pressure of an ejecting fluid. FIG. 38B shows similar cover flaps that are angled to direct the ejecting fluid creating a circular motion.

FIGS. 39A and 39B show the distal tip of a catheter element with cover flaps folded to the interior lumen of the catheter.

FIGS. 40A and 40B show the distal tip of a catheter with flaps attached to the catheter on the bottom, or proximal edge of the ports. These flaps could be more rigid in nature to direct fluid in the desired direction.

FIGS. 41A, 41B, 41C illustrate a mechanism that could be implemented in the distal tip of the catheter to regulate the flow patterns distal and proximal of the tip. FIG. 41A shows fluid escaping from the distal end of the tip. FIG. 41B shows fluid pressing against a lever to close the distal ports and open the side ports of the tip. FIG. 41C shows a system similar to that in A and B expanded to incorporate multiple such ports.

FIGS. 42A and 42B show a construction technique that would allow for the manufacture of small scale ports with directionality of flow. FIG. 42A shows a catheter with a section cut out of its side near the distal tip. FIG. 42B shows that same catheter with a small cover placed over a portion of the hole, lending directionality to the fluid to be ejected.

FIGS. 43A, 43B, 43C, 43D are top view cross-sectional diagrams to show some variations of rinse tips that would result in a circular or rotational flow of either the fluid or the distal end of the catheter or both.

FIG. 44 shows a cross-sectional top view of a rinse head with the addition of relief areas of detail designed to enhance the fluid flow surrounding the catheter.

FIG. 45 shows another cross-sectional top view of the distal tip of the catheter where the details are slight protrusions designed to enhance the fluid flow around the catheter.

FIGS. 46A and 46B show a tip for a catheter that would allow for rotation driven by the ejecting fluid. FIG. 46A is a cross-sectional side view illustrating a joint that would allow for rotation of the tip. FIG. 46B is a side view showing the ejection ports that would expel fluid with some directionality.

FIGS. 47A and 47B show a tip with channels arranged to tend to eject fluid more in the distal direction.

FIG. 48 shows a cross-sectional top view of a vessel interior with the catheter inside it. The arrows indicate a possible desired fluid flow that could be created by the arrangement of the rinse head.

FIG. 49 is a side view diagram with the distal end of the catheter at the top of the page. This diagram shows a possible path of a molecule of rinsing fluid. This fluid flow pattern would be induced by the design of the irrigation ports.

FIGS. 50A, 50B, 50C, 50D show side views of the distal tip of the catheter with a mechanism for opening and closing the ejection ports. A piece of compressible material is the main component of this passive gate device. FIG. 50A shows the compressible material covering the ejection ports. FIG. 50B shows the compressible material pushed distal and out of the way by the force of the fluid. FIG. 50C shows a cross-section of A. FIG. 50D is a cross-section of B.

FIGS. 51A and 51B are cross-sectional views of the distal tip of the catheter showing a simple spring mechanism used to activate a closure system for the fluid ports. FIG. 51A shows the spring in its natural state, extended and thus positioning a plug to block the ports. FIG. 51B shows the spring compressed by the fluid and therefore positioning the plug distal to the ports, thereby allowing fluid to escape.

FIGS. 52A and 52B are cross-sectional side views of the distal tip of the catheter showing a threaded plug that could be opposed by an optional compressible material to allow for gating of the ports.

FIG. 53 is a simple cross-section illustrating that any of the compressible materials referred to could be layer of differing materials or densities to yield the desired material response.

FIGS. 54A and 54B are side views of the distal tip of the catheter showing a variation of the port geometry to achieve desired fluid flow response. FIG. 54A has the port getting more slender at the distal tip. FIG. 54B has the port widening toward the distal tip.

FIGS. 55A, 55B, 55C, 55D are side view cross-sections of the distal tip of the fluid introduction catheter showing an arrangement that would allow for a pulsing flow of the ejection fluid. FIG. 55A shows the catheter's default arrangement with the side balloon deflated and the plug in the proximal position and the ports covered. FIG. 55B shows the side balloon filling with fluid as pressure builds within the catheter. FIG. 55C shows the plug moving distally as the pressure has increased enough to overcome the resistance of the spring (or equivalent). FIG. 55D shows the tip in the process of ejecting fluid with the additional pressure provided by the side balloon. The system would then return to its default position and the cycle would continue.

FIGS. 56A and 56B are cross-sectional side views of the distal tip of the catheter combined with a balloon that could be used for purposes similar to those of angioplasty. FIG. 56A shows both balloons inflated. FIG. 56B shows the inner balloon deflated and the outer balloon full of fluid which is being ejected through holes in this case placed on the proximal half of the balloon.

FIGS. 57A and 57B show side views of the distal tip of the fluid introduction catheter within a vessel. FIG. 57A is a single lumen catheter which ejects fluid both distally and proximally through ports in accordance with their geometries. FIG. 57B shows a two-lumen catheter allowing for greater differentiation of the amount or type of fluids being ejected.

FIG. 58 is a side view of the distal tip of the catheter showing the addition of a direction-giving channel at the very distal tip to help guide the fluid exiting there.

FIGS. 59A and 59B are side views of the distal tip of the fluid introduction catheter illustrating a system for selectively allowing fluid to escape through the distal tip of the catheter using in internal balloon. FIG. 59A shows the balloon deflated and fluid passing through the very distal port. FIG. 59B shows the internal balloon inflated, thereby blocking fluid flow to the distal most port.

FIGS. 60A and 60B are side views showing the combination of the catheter with an angioplasty catheter. FIG. 60A shows the angioplasty balloon inflated. FIG. 60B shows the angioplasty balloon deflated before or after the treatment and the fluid introduction catheter ready to dispense fluid.

FIGS. 61A and 61B are side views illustrating the combination of the catheter with a stent deployment device. FIG. 61A shows the arrangement in its low-profile, insertion state. FIG. 61B shows the stent deployed and the fluid introduction catheter ready to apply fluid to the region.

FIGS. 62A and 62B are side views of the distal tip of the catheter showing a configuration to send fluid significantly in the distal direction. FIG. 62A shows the device in its low-profile, insertion state. FIG. 62B shows the device deployed, the cover flap expanded by the pressure of the ejecting fluid, with the cover flap sending fluid largely in the distal direction.

FIG. 63 is a side view showing the distal end of the catheter. Here is a construction which allows for the direction of fluid in both the distal and the proximal direction. The multi-lumen construction allows for different fluids to be sent in the two directions.

FIG. 64 is a side view showing the distal end of the catheter. This arrangement shows two flap structures, one for the distal and one for the proximal. Multiple lumens allow differentiation of the fluids sent proximally and distally.

### DETAILED DESCRIPTION OF THE INVENTION

The present invention may be used in a number of different environments and for a variety of purposes including, but not limited to all physiological uses of peristaltic or other pump for aspiration and irrigation including, IVUS, OCT, angioplasty, endarterectomy, cardiac stent placement, vessel treatment, diagnosis and repair, surgical placement of non-cardiac stents, insertion of pig-tail catheters, ear rinsers, etc. The following detailed description is exemplary of possible embodiments of the invention.

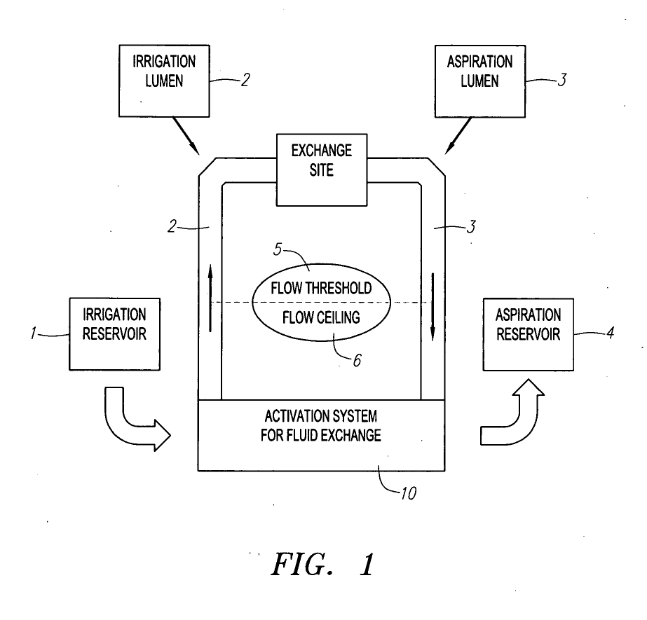

Referring to FIG. 1, a schematic representation of the invention shows the basic components of the device necessary for implementation. The core fluid exchange or activation system maintains a substantially closed loop system with the target site for fluid exchange, e.g. the site within the body where aspiration and irrigation are applied. The irrigation component of the invention is conveniently provided by a dedicated irrigation reservoir 1, particularly when the fluid exchange system is the mechanical embodiment described in greater detail below. The exchange site is in fluid communication with the fluid exchange system via the irrigation lumen 2 and the aspiration lumen 3 which have exit or entry ports (not shown) at the distal end of each lumen. The aspiration component may also feature an aspiration reservoir 4 in fluid communication with the aspiration lumen 3 and aspiration ports (not shown) such that fluids removed from the exchange site are stored in the aspiration reservoir 4. As is apparent to one of ordinary skill in the art, the irrigation 1 and aspiration 4 reservoirs may be controlled electronically by valves or pumps to provide the controlled fluid exchange ratios described herein. Thus, while the embodiments of the invention featuring fluid exchange apparatus that are mechanically controlled by the user are preferred in certain versions of any system, controlled rate of fluid exchange at a target site may be used in a system of the invention. Alternatively, fluids in the aspiration reservoir 4 may be discarded. In one embodiment of the invention, fluids communicated from the target exchange site through the aspiration component of the invention are analyzed for chemical or particulate content to determine a level of removal of fluids or solid matter from the exchange site.

Referring again to FIG. 1, an optional configuration of the components includes a flow valve 6 that produces a minimum lower threshold for irrigation flow. This minimum delivery flow is beneficial to ensure a minimum amount of exchange flow when the clinical indication dictates maintaining a minimum flow through the irrigation catheter. The flow threshold insures that the fluid exchange does not fall below a predetermined ratio as described herein. For example, although 1:1 fluid exchange rates are provided in several embodiments described herein, the exchange ratio may be altered such that a larger volume of fluid is aspirated compared to that which is used for irrigation or vice versa. Under such circumstances, the fluid exchange ratio would vary to, for example, a 1:2 irrigation to aspiration ratio under circumstances where a larger volume of liquid is desired to be removed from the exchange site.

The components of the invention could also incorporate an upper flow rate of exchange or flow ceiling 6. When conditions dictate that there is motivation to limit the velocity or overall flow parameters during a usage, a configuration that provides an upper limit may be provided. Accordingly, this embodiment would apply where a larger volume of fluid was desired to be inserted by irrigation compared to that which is removed by aspiration and the corresponding irrigation to aspiration exchange ratio would be increased to, for example, 2:1. The combination of a flow threshold and flow ceiling capability provide a flow rate bandwidth yielding a range of values between two extremes. In this embodiment, the exchange site can be irrigated and aspirated at a consistent level that is either fixed or varies within a range. This may also allow the activation system to sustain a change in the pressure level at the exchange site while delivering irrigant fluid or removing aspirant fluid at a steady rate or within a range of rates. As will be appreciated by one of ordinary skill in the art, the irrigation side of the system of the invention requires active force provided by the fluid exchange apparatus such that irrigant fluid flow is established at the target site. However, while the aspiration side may also be controlled through application of force to withdraw fluid from the target site, the aspiration side may also be passive such that the inherent pressure at the target site propels the aspirant fluid. The inherent pressure is typically provided both by the fluid pressure inside the body, e.g. the blood pressure within a vessel, and the pressure of the irrigant fluid entering the target site. This characteristically passive flow may be described as an efflux flow, see U.S. Pat. No. 4,921,478 which is specifically incorporated by reference herein. The passive flow of aspirant fluid is one way through the aspiration lumen and the fluid pathway is comprised of one-way valve, such as conventional duck bill valves having a minimal cracking pressure to allow passive fluid flow while preventing retrograde flow through the aspiration side of the system. This capability provides for constant extraction of embolic particles throughout a clinical procedure while irrigant fluid flow is maintained and/or when fluid existing at the target site flows from endogenous body pressure.

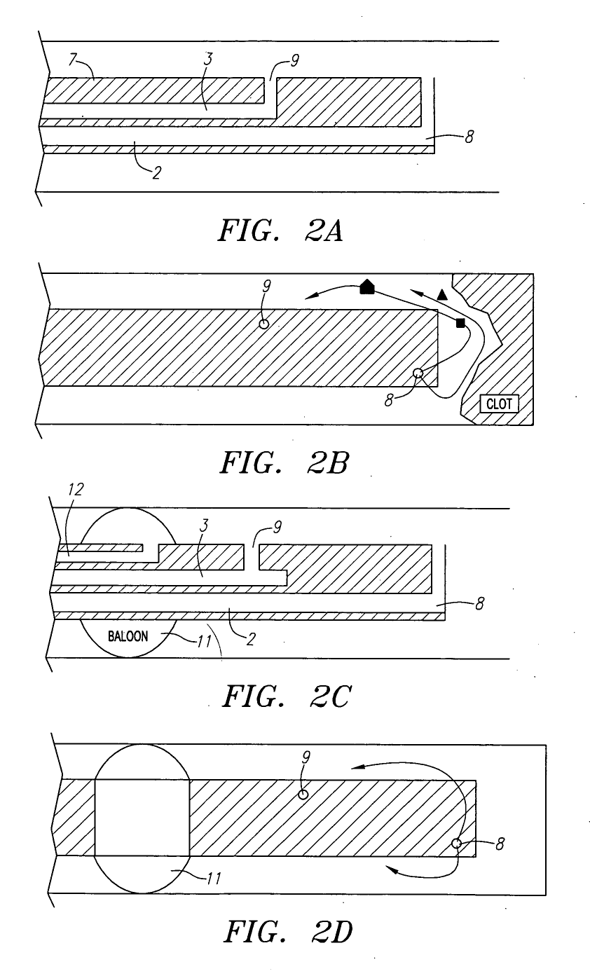

FIG. 2A is a cross-section of a catheter element 7 of the invention at the exchange site. The irrigation lumen 2 in this configuration terminates at or proximate to the distal end of the catheter element. While the aspiration lumen 3 terminates proximally and both lumens terminate with exit ports 8, 9. FIG. 2B depicts the insertion of fluid into an exchange region at a terminal lumen. The irrigation port 6 in this depiction is dislodging a terminal occluding clot. The terminal occlusion may include but is not limited to a clot, lesion, abscess, a ball of wax or an ear canal. In such situations, simple aspiration may not eliminate the lesion and a non-traumatic irrigation of the lesion with a therapeutic formulation, in concert with aspiration after an improved treatment methodology. For example, even if the irrigation fluid is able to produce a substantial breakdown of a terminal occlusion, the occlusion itself must still be cleared. Moreover, the combination of irrigation and aspiration to yield fluid exchange after the ability to introduce pharmaceutical agents proximate to the occlusion and the ability to remove the agents before they enter the bloodstream. A specific example of this is a thrombolytic agent used to remove the occlusion or potentially dangerous thrombus, wherein the thrombus or occlusion must be both treated and removed to treat the condition and wherein the necessary dosage of the agent exceeds that which could otherwise be introduced without drug-related toxicity.

FIG. 2C is a cross-section of the catheter element of the system incorporated with a proximal occlusion balloon 11 to establish a defined region of fluid exchange. This configuration may be useful for, but is not limited to, occluding flow, limiting a diagnostic agents field of deployment or limiting the bodies exposure to a high intensity agent. A dedicated balloon lumen 12 is provided for inflation of the occluding device. FIG. 2D is the catheter element of the system of the invention having an occlusion member 11 to aid in establishing an exchange site and having irrigation and aspiration functions distal to the occluding member wherein the arrows depict the general direction of fluid flow, distal to proximal, relative to the occluding member 11. In certain situations, it is neither convenient nor necessary to use a proximally placed occluding balloon while performing simultaneous infusion and aspiration as in FIG. 2C. Instead, it may be desirable to merely increase the resistance to flow along the region surrounding (outside of) the irrigation and aspiration lumens proximal to the site of rinsing and aspiration. This can be performed by incorporating one or more balloon members (not shown) that expand only during activation of the flow of irrigation solution, but which deflate automatically. The automatic deflation can occur by means of having an intentional leak in the system comprising the balloon lumen 12 that inflates the balloon(s) such that when they are not actively being inflated, their default state is to collapse by forcing fluid out of the balloon lumen 12 via one or more orifices (not shown). The extent of the rate of pressure release through these orifices may be controllable via a valve to accommodate for different clinical applications and/or anatomic variants. The “leaks” or orifices can exist anywhere within the region in fluid communication with the balloon lumen 12, such as the balloons 11 themselves, or more proximally, at the proximal end of the balloon lumen 12. These balloons 11 do not necessarily have to be large enough to fill the entire region/vessel that they reside in to achieve the desired effect of further localizing the aspiration of fluid to that fluid situated generally distal to the catheter element 7. Such a design may be useful in the delivery of rapidly-acting thrombolytics to sites of thrombus in acute myocardial infarctions and/or strokes. Such a system would provide a method to locally deliver and remove fluids in the region near the distal end of the irrigation lumen 2 and aspiration lumen 3, yet would have a safe default state of being in the deflated state, thus minimizing the time during which the tissues downstream of the balloons 11 do not receive perfusion.

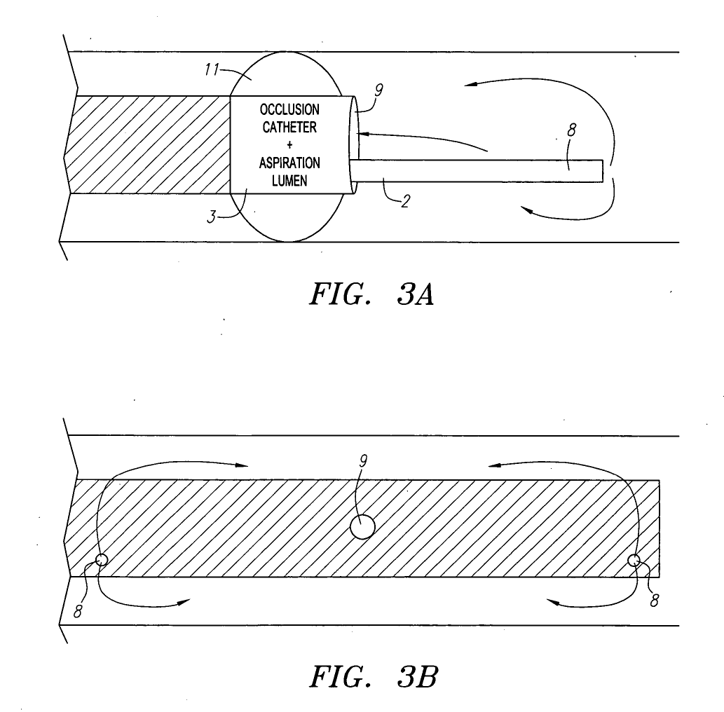

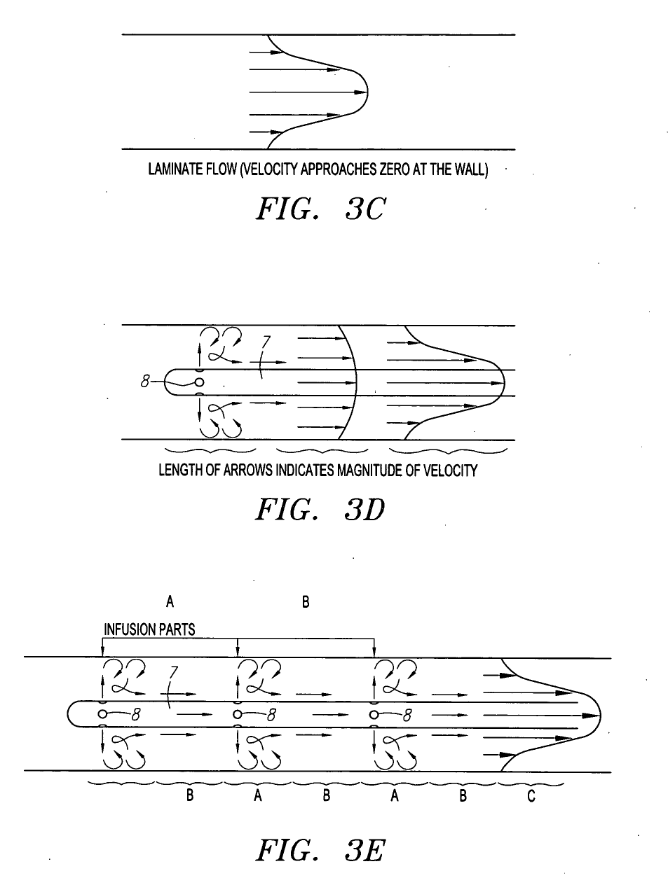

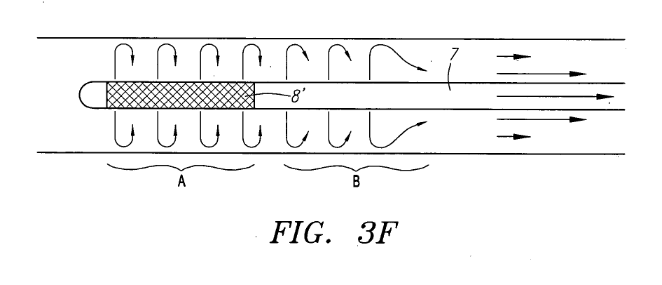

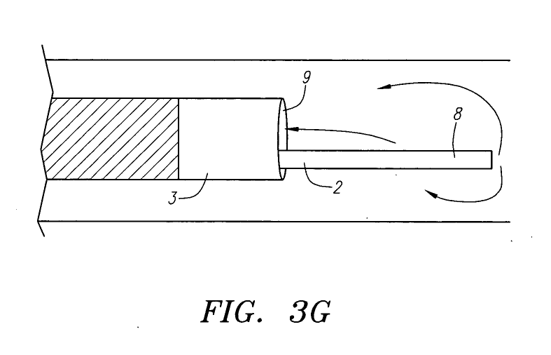

FIG. 3A is the device incorporated with a combined aspiration lumen 3 and occluding element 11 integral in the same catheter element with the irrigation driven by a separate catheter 2 to aid in defining a target site or field of fluid exchange. The irrigation lumen's 2 independent travel affords a means of adjusting the location of the fluid exchange site while maintaining the occlusion at a predetermined location. Furthermore, a treatment, diagnostic or imaging tool (not shown) can also be affixed to the irrigation catheter 2. This is productive where the resident fluids are desired to be replaced with a dye or contract agent and then removed in turn prior to re-establishing normal blood flow. In optical coherence tomography (OCT), for example, it is advantageous to introduce and remove a low attenuating fluid. FIG. 3B is a fluid isolated region that is maintained by irrigation occurring through ports 8 located both proximal and distal to the aspiration port 9. This configuration presents a means of maintaining a controlled introduced field of fluid between the proximal and distal irrigation ports 8. As in the embodiment of FIG. 3A, a treatment, diagnostic or imaging tool could be attached or moved along in concert between the irrigation ports. Referring to FIG. 3C, a catheter element (not shown) that merely inserts and removes fluid from a vessel achieves only laminar flow in the direction of the arrows and with velocity illustrated by the size of the arrows. Near the vessel wall the total fluid flow approaches zero such that fluid containing emboli at the walls is not disturbed and loosely affixed emboli remain in place. FIG. 3D is a preferred embodiment of the catheter element of the invention having orthogonally disposed aspiration ports 8 located at the distal end of the catheter element 7. The region “A” experiences turbulent flow, while region “B” experiences a flow profile that is in transition from turbulence to laminar flow. FIG. 3E shows a series of irrigation ports 8 spaced at intervals along the length of the distal end of a catheter 7 such that either turbulent flows, designated as “A” or regions where turbulence is transitioning to laminar flows, designated as “B” are established along a length of the catheter 7 to substantially eliminate areas of laminar flow. FIG. 3F shows a configuration wherein the irrigation ports are provided as a perforated region 8′ at the distal end of the catheter 7. The arrows indicate the direction and magnitude of flow showing that the perforated region establishes turbulence in a defined region, and as the distance away from the perforated portion 81 increases, the flow reverts to a laminar flow at a certain distance along the length of the vessel. FIG. 3G shows an embodiment useful in clinical circumstances where a two-catheter system for fluid exchange is advantageous that is similar to FIG. 3A without the occlusion. In this embodiment, a catheter comprising an irrigation lumen is advanced concentrically through another, larger aspiration catheter comprising an aspiration lumen. This second catheter can be considered similar to the occluding catheter described elsewhere, but without the occlusion. These catheters are attached to the fluid exchange system and fluid is irrigated and aspirated at a ratio between 1:1 and 1:3. In a preferred embodiment, the system would irrigate and aspirate in a 1:2 ratio. The rinsing catheter would deliver the irrigation fluid distal of the sight of interest. Simultaneously, the aspiration catheter 3 would remove twice as much fluid proximal of the site of interest.

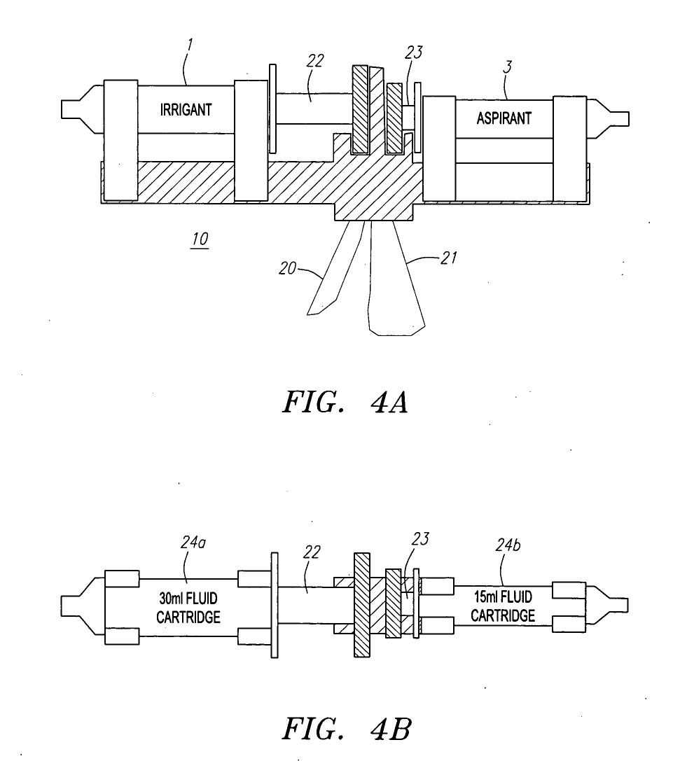

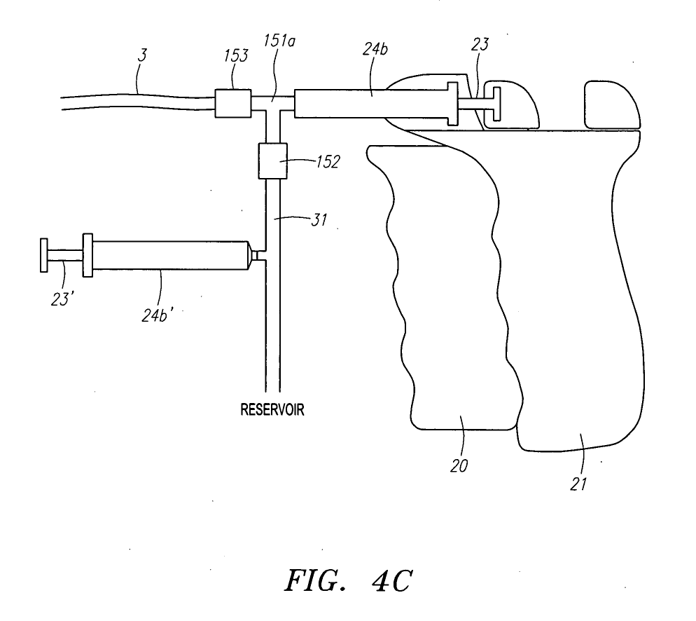

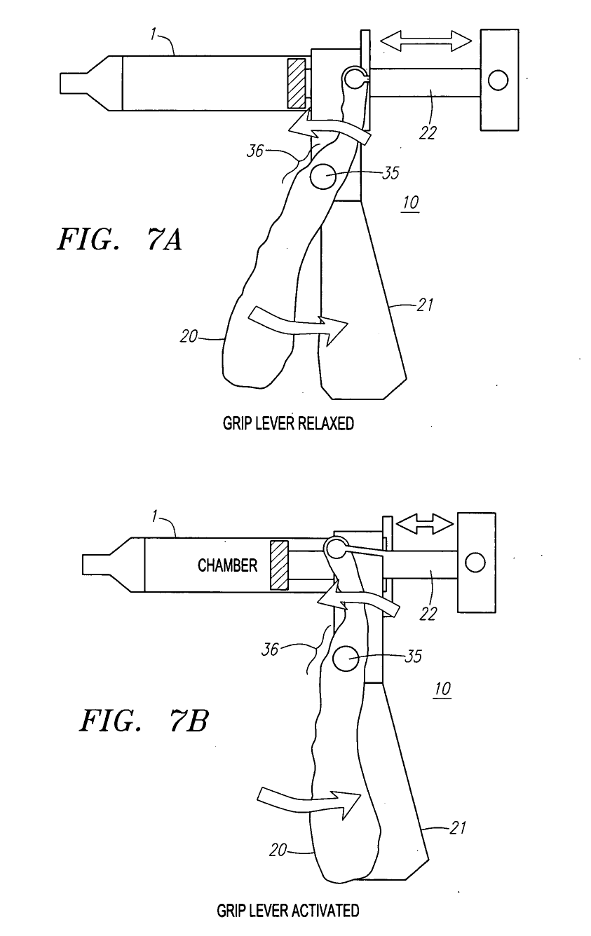

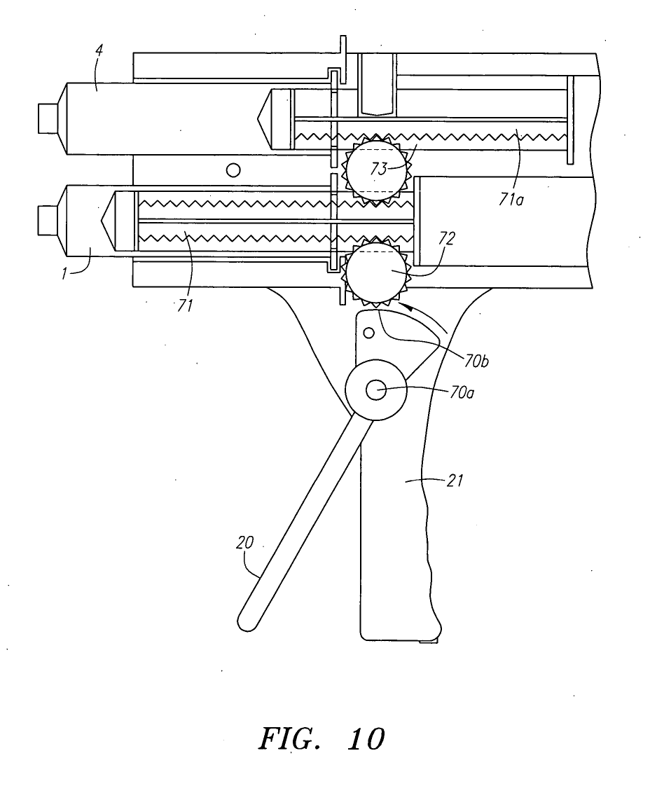

FIG. 4A is an embodiment of the device 10 that produces pulsatile flow through the application of a mechanical force to an apparatus that propels fluid through the catheter element of the invention. In use, the action of a trigger 20 pulled toward a handle 21 exerts a force on a dedicated irrigant piston 22 that compresses the irrigant reservoir 1 thereby reducing the volume of the irrigant reservoir 1 and forcing fluid through the irrigant lumen (not shown) and simultaneously withdraws the dedicated aspirant 23 piston of the aspirant reservoir 4 to accomplish the fluid exchange at the target site. Actuation of the trigger 20 may cause the relative motion of the pistons 22, 23 by connection handle to a ratchet or other gear mechanism that provides the exertion of force in an incremental amount based on the actuation of the handle in a cyclical fashion. See e.g. FIG. 10 below and accompanying text. As shown in FIG. 4A, the irrigant and aspirant reservoirs may advantageously be provided by conventional syringes or similar devices that provide for fluid containment and the controlled application of fluid flow. The syringes of FIG. 4A are merely examples of the use of replaceable cartridges that may be readily inserted and removed from the device. Such cartridges are particularly useful when pharmaceutically active solutions are pre-filled and used in specific clinical procedures where medicaments are provided to a body conduit or vessel by the system of this invention. In this respect, the use of this invention allows the selective introduction of pharmaceutical compositions of any type during the performance of an ordinary irrigation and aspiration operation. In the embodiment of FIG. 4A, the syringes comprising the irrigant reservoir 1 and aspirant reservoir 3 may be removably inserted into the hand-held fluid exchange apparatus 10 and used to both provide and expel a predetermined volume of fluid through the target exchange site. In this manner, both the volume and content of the irrigant fluid can be controlled by exchanging syringes and the contents of the aspirant reservoir can be retained and analyzed for fluid or particular content. The operation of preferred embodiments of the hand-held embodiment of the invention is also described at FIGS. 7-10 below and the accompanying text.

FIG. 4B is an example of interchangeable fluid cartridges 24a 24b, similar to the syringes described in other embodiments, for irrigation and aspiration. As described in greater detail herein, the irrigant 1 and/or aspirant 3 fluid reservoirs may be provided by cartridges or reservoirs of differing sizes to accomplish the predetermined volume exchange ratio desired for the particular clinical indication. In the embodiment of FIG. 4B, the irrigant fluid cartridge 24a has double the volume of the aspirant cartridge 24b thereby providing a 2:1 fluid exchange ratio of irrigant to aspirant at the target site. In this respect, the loop established by the fluid exchange system is not a completely closed loop, but is described as a substantially closed loop, in that a difference exists between the volume expelled through the irrigant reservoir 1 via the irrigant lumen 2 and into the exchange site versus the difference in the aspirant volume taken up through the aspirant lumen and into the aspirant reservoir 40 although the volumes are not identical, the volumes are predetermined and known with certainty as is the volume of fluid that remains at the target site, which is the difference between the volume of the irrigant fluid introduced to the site and the volume of the aspirant fluid removed therefrom. As in the embodiment of FIG. 4A, the irrigant fluid cartridge 24a has a dedicated piston 22 for expelling fluid from the cartridge. The aspirant cartridge 24b similarly has a dedicated piston 23 for collecting fluid introduced to the aspirant reservoir via the aspiration lumen 3. In this specific embodiment, more irrigant fluid is introduced due to the larger cross-section of the irrigant cartridge 24a while the overall length of the cartridge that fits into the fluid exchange apparatus remains constant. This technique for providing varying fluid cartridge volumes is advantageous when the irrigant and aspirant cartridges are replaceable in a fluid exchange device.

Referring to FIG. 4C, an added fluid flow component having a fluid reservoir, chamber or other mechanism to promote fluid flow can be inserted on either of the irrigation or aspiration side of the fluid conduits of the invention. For example, where the intent of a therapeutic procedure is to withdraw emboli from a site known to contain a high risk of emboli generation, an added fluid flow component such syringe or equivalent can be attached to be in fluid communication with the aspiration side of the system. In this embodiment, the supplemental aspiration syringe removes an additional amount of fluid from the aspiration side, wherein the amount can be predetermined or selected at the operator's discretion. Of course, the fluid flow component 246 can be inserted at any point along the aspiration side, and preferably has one-way valves 152, 153 to prevent infusion of material in a retrograde direction. The system includes a branched lumen 151 a to facilitate attachment of the component 246 and the one-way valves 152, 153. Typically, the additional fluid aspirated in this manner is in addition to the fluid exchange or fluid recirculation achieved with the remaining components of the system and provides the operator/clinician with the ability to continually alter the fluid flow parameters.

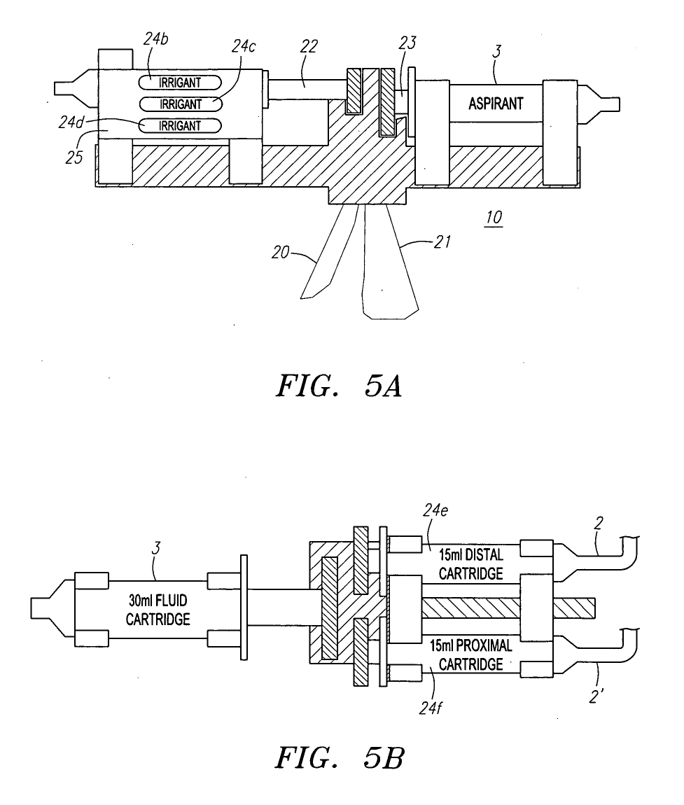

FIG. 5A is a revolving cartridge 25 that can rapidly provide a series of irrigant solutions. This revolver-style orientation of irrigant solution is advantageous for delivery of a sequence of different fluids or for delivery of a pharmaceutical composition at an intermediate point during a procedure. In use, the revolving cartridge 25 is oriented such that the series of irrigant fluids 24b, 24c, 24d are positioned in line with the dedicated irrigant reservoir piston 22 to expel the selected irrigant solution placed in line with the piston 22. Under certain clinical circumstances, the application of the system of the invention may provide an ordinary rinsing solution such as saline at the beginning of a procedure to clear resident fluids and/or emboli from a site, followed by the introduction of a pharmaceutical solution, followed by the removal of the pharmaceutical solution and the subsequent introduction of a neutral solution. In such a use, the saline solution would be confined in the first irrigant reservoir 24b, which would be infused by actuating the handle 20 as in the embodiment of FIG. 4A described above. Subsequently, the contents of the second irrigant reservoir 24c, such as a thrombolytic agent, dye, contrast agent or other formulation, is inserted by rotating irrigant reservoir 24c in line with the irrigant reservoir piston 22, and similar actuation of trigger 20. Once the desired effect provided by the solution of reservoir 24c has been achieved, the solution may be rinsed from the vessel by rotating the dedicated irrigant reservoir 24d into place and actuating the fluid exchange system as above. Similarly, a variety of aspirant chambers (not shown) can be used to facilitate collection and testing of the aspirant fluid by segregating discrete volumes into containers that can be processed for analysis.

FIG. 5B is an embodiment where two different irrigant fluids can be delivered at equal time and measure in a pair of cartridges 24e, 24f that are designed to be delivered through one or a pair of irrigant lumens 2, 2′ such that one irrigant lumen 2 delivers fluid distal to a predetermined point at the target site and the other irrigant lumen 2′ delivers fluid proximal to a predetermined point at the target site. In such a case, each of the two irrigant lumens 2, 2′ has a dedicated irrigant port or ports located at the distal end of the catheter element. The division of the irrigant reservoir 1 into two components 24e, 24f allows for the selective introduction of irrigant fluids, which may be the same solutions or different solutions at two or more points within the target site. The predetermined-point in the target site that separates the proximal and distal delivery of irrigant fluid may be an aspirant port located therebetween (as in the embodiment of FIG. 2D) or any other structure where separation of irrigant fluid is desired. For example, some irrigants may mix advantageously only at the exchange site and could not be combined outside the body based on their chemical reactivity.

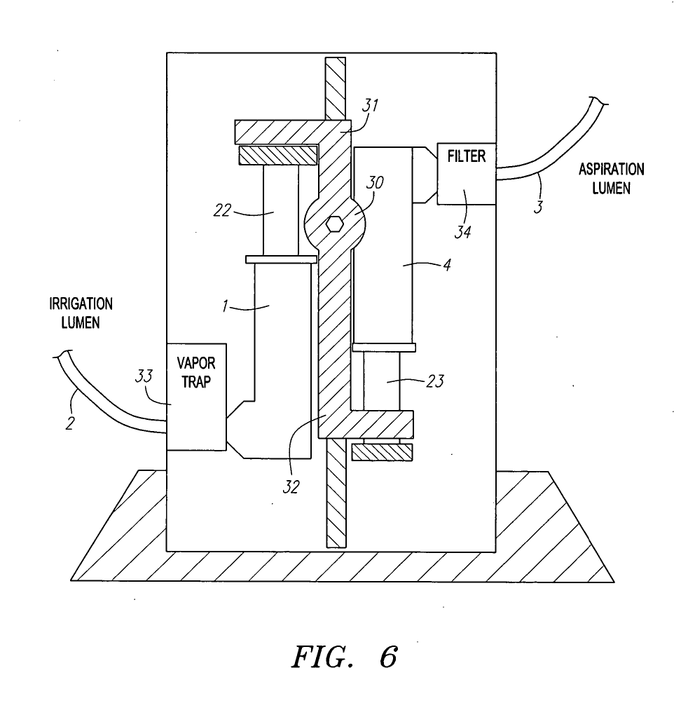

FIG. 6 is a tabletop version of the fluid exchange device of the invention. As is described elsewhere herein, the fluid exchange apparatus of the invention may be controlled by the simple mechanical operation of a device by a user or by an electronic system that controls a mechanical or electrical pump- or valve-driven system to control the irrigant 1 and aspirant 4 reservoirs. In the embodiment of FIG. 6, a variable position lever 30 drives the stroke of a dedicated piston 22, 23 that forces fluid from the irrigant reservoir and draws fluid into the aspirant reservoir. As with the embodiments described above, the cycle and the volume of the reservoirs or motion of the pistons can be altered to match the fluid exchange volume needed for any flow in the vessel or body conduit. Because the rotation of the individual levers is variable, the ratio of fluid exchange can be achieved by different positioning of the lever arms 31, 32 rather than by altering the volume of the individual irrigant 1 and aspirant 4 reservoirs. Although this embodiment shows the mechanical application of force through levers, a tabletop version of the apparatus of the invention is advantageous when electronically controlled pumps are provided to control the fluid exchange and fluid exchange ratios. The embodiment of FIG. 6 also may include an in-line air trap 33 for the irrigant reservoir 1 and/or a filter 34 for the aspirant reservoir 4. As it may be advantageous to eliminate debris upon extraction of irrigant fluid and/or prevent air upon entry of irrigant fluid, the inclusion of a filter or trap 33, 34 for air and/or emboli is appropriate in some cases.

FIGS. 7A and 7B show the internal structure and function of a fluid exchange device 40 where a pair of reservoirs control fluid flow via the force exerted by pistons or plungers following the action of a trigger 20 and handle 21 connected to or integral with a lever 36 that rotates about a pivot 35. In this embodiment, the actuation of the trigger 20 rotates the level 36 about pivot 35 and forces the irrigant reservoir piston 22 into the irrigant reservoir 1 and simultaneously withdraws the aspirant reservoir piston 23 out of the aspirant reservoir. From the relaxed position (FIG. 7A), the trigger 20 can be activated to drive the pistons 22, 23 through either a direct coupling or a mechanism for incremental cycles. If desired, the trigger 20 can return to the relaxed position after a cycle from spring action in the handle or pivot 35 other automatic return mechanism. The reservoirs may be integral to the device 10 or the volume of the reservoir 1 may be attached to a separate reservoir (not shown) together with the appropriate lumens, and preferably in-line one-way valves, to facilitate the exchange between the separate reservoir and the chamber of the device. In the former embodiment, the reservoirs are integral to the handle-operated device such that the piston exerts a direct force on the irrigant 1 and/or aspirant 4 reservoir to exert the force necessary for fluid exchange. In the above embodiment, the internal structure of the device acts as an in-line chamber that is intermediate between the separate reservoir and the lumen such that irrigant fluid residing in a separate reservoir is drawn into the chamber prior to being expelled from the chamber through the irrigation lumen. In this embodiment, a pair of lumens are required, a first intermediate lumen connecting the separate reservoir to the chamber, and a second lumen communicating the irrigant fluid from the chamber through the irrigant lumen and via the irrigant ports to the target exchange site.

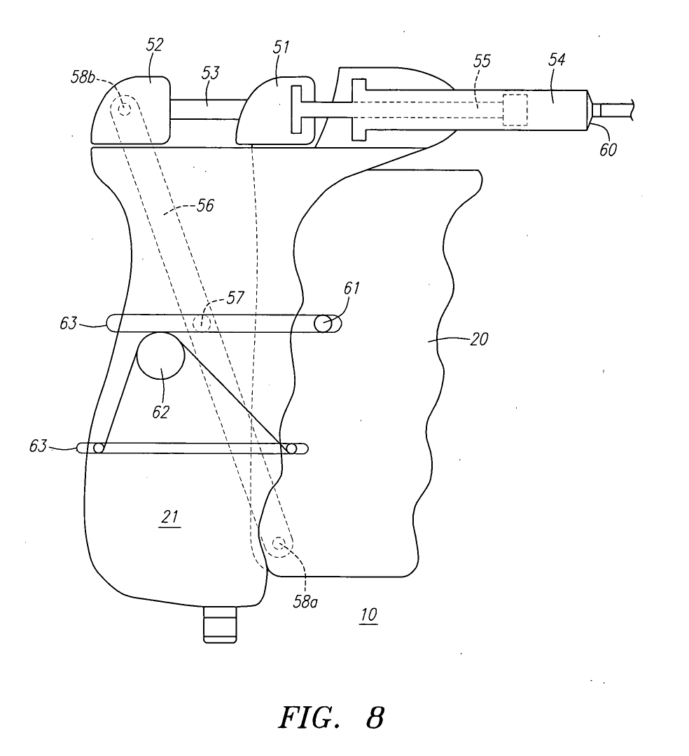

FIG. 8 is a preferred embodiment of the invention having a trigger 20 that is squeezed by the hand to operate a syringe that acts as the aspirant reservoir 54 and the irrigant reservoir (not shown). As the trigger 20 moves toward the body of the handle 21, the force is transmitted both to the piston 55 dedicated to the aspirant reservoir 54 and a separate piston (not shown) dedicated to the irrigant reservoir. Although the internal configurations can be varied to incorporate other mechanical expedients, the orientation of the lever 56 and pivot 62 of the present embodiment provide an advantageous mechanism for a 1:1 ratio fluid exchange. The action of trigger 20 is communicated to a lever 56 that is connected to the trigger 20 by a first terminal lever connector 58a. When the trigger 20 moves toward the body of the handle 21, the force exerted on the lever 56 rotates the lever 56 around pivot 57 to exert a force, via a second terminal lever connector 58b that is attached to an irrigant carriage 52. Simultaneously, the motion of the trigger 20 exerts force on a second lever (not shown) that is connected to the aspirant carriage 51 in a similar matter as for the irrigant carriage 52. The motion of the trigger 20 provides a simultaneous but opposite force on the aspirant cartridge 51 compared to the irrigant cartridge 52. The simultaneous forces that are applied to the pistons dedicated to the irrigant reservoir and aspirant reservoir 54, respectively, occur in opposite directions to yield a substantially equivalent volume exchange into the aspirant reservoir 4 and out of the irrigant reservoir 1 via the aspirant and irrigant lumens 4, 2 respectively. The motion of the irrigant carriage 52 is translated to the piston dedicated to the irrigant reservoir by virtue of a connector 53 that is noncompressible and that is aligned with the length of the irrigant reservoir 1.

As noted specifically with the embodiments described at FIG. 4A herein, the irrigant and aspirant reservoirs 1, 4 may be interchangeable syringes or cartridges that can be inserted and removed to introduce specific solutions or fluid volumes. In a preferred embodiment, the irrigant and aspirant reservoir 1, 4 may be molded into the body of the device such that the fluid volumes for the irrigant and aspirant reservoirs are separately filled via a fixture that acts as an input valve to the irrigant and/or aspirant reservoir. The irrigant and aspirant reservoirs 1, 4 preferably have removable fixtures at the output 60 thereof for attachment of the respective lumens 2, 3.

The motion of the trigger 20 is rendered linear and reproducible by slots 61 cut into a portion of the trigger 20 that are engaged by the first pivot 57 and the second pivot 61 such that the body of the handle 21 and/or the trigger 20 slidingly move about either of the pivot structures. A second lever 63 operates parallel to the lever 56 to enable the trigger 20 to travel smoothly along its path. This configuration provides for reproducible motion of the trigger 20 relative to the body of the housing 21 and also facilitates attachment of a spring 62 that biases the trigger in the forward position so that actuation of the trigger 20 relative to the handle 21 produces a complete cycle that translates into a defined movement of both the irrigant cartridge 52 and the aspirant cartridge 51. The volume exchange ratio provided by the device of this invention may be altered by changing the relative lengths of the lever 56 relative to the pivot 57 or by altering a ratcheting mechanism disposed at the connection point between the lever 56 and the irrigant cartridge 52 such that a complete cycle of the trigger 20 from the forward most position when moved toward the body of the handle 21 constitutes a complete cycle that moves the irrigant 52 and/or aspirant cartridge by fixed distance. The spring tension automatically returns the trigger 20 to the forward most position to prepare for a second cycle.

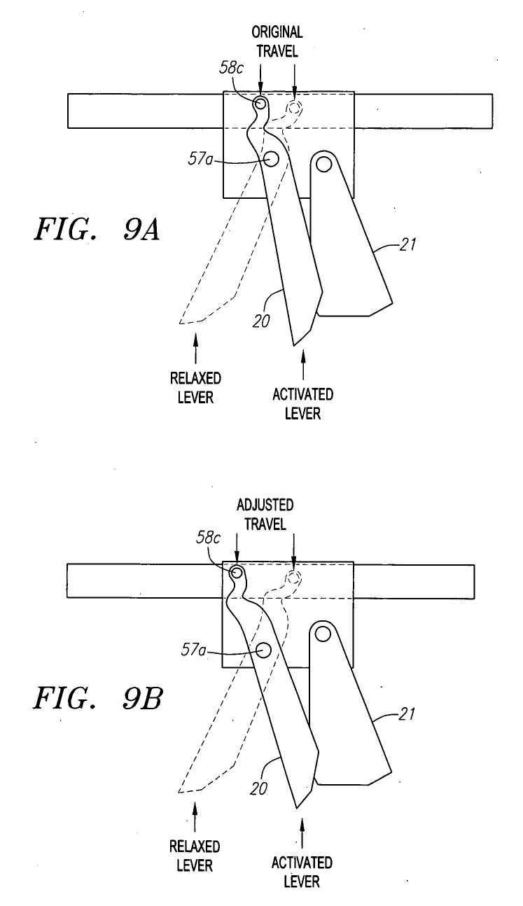

FIG. 9A is an embodiment where the travel of the lever in the fluid exchange device is adjustable so that the amount of fluid displaced in a single cycle can be controlled, and both the distance traveled and the force generated can be adjusted by relative positions of the trigger 20 and the handle body 21. The embodiments of FIGS. 9A and 9B illustrate the ability to alter the fluid flow parameters of the fluid exchange device by changing the configuration of the mechanical components that exert force on the irrigant reservoir 1 and aspirant reservoir 4, respectively. FIG. 9B illustrates the adjustment of the pivot point 57a to produce different flow ratios and peak pressures based on the relative position of the pivot point 57a about which the trigger 20 rotates. In such an embodiment, if more fluid flow is desired the apparatus can be easily adjusted to accomplish a variable number of flows for a given grip cycle. The travel distance provided by the motion of the trigger 20 as exerted at the point of attachment by the second terminal lever connector 58c dictates the amount of fluid flow expelled from the irrigant and/or aspirant reservoir 1, 4 based on the action by a syringe or aspirant reservoir piston or carriage as described above. Accordingly, an increase in the motion of a piston compressing fluid in an irrigant or aspirant reservoir or chamber, due to changing the pivot point, results in an increased exchange rate for a given activation of the trigger 20. As is shown in FIGS. 9A and 9B, the adjustment to the degree of travel of the trigger 20 relative to the handle 21, when combined with aspiration 51 and irrigant 52 carriages and reservoirs as described in, for example FIG. 8 above, produces the variable fluid flow of this embodiment. As with the embodiments described above, the mechanical movement of the trigger 20 relative to the handle 21 is translated into fluid flow from an irrigant reservoir 1, via irrigation lumen 2, aspiration lumen 3, and aspirant reservoir 4 by the configurations described herein.

FIG. 10 is a hand-held fluid exchange apparatus of the invention wherein a ratchet mechanism provides for incremental movement of a piston, in this embodiment, a general set of pistons 71, 71 a for driving fluid out of the irrigant reservoir 1 and into the aspirant reservoir 4, respectively. As in the embodiment of FIG. 8, the motion of a trigger 20 relative to a body handle 21 completes one cycle. This embodiment may also contain a mechanical or electrical counter that provides a readout indicating the number of cycles that have been performed, the volume of fluid introduced or removed, or the amount of fluid present, or remaining in either reservoir. In this embodiment, the motion of the dedicated, geared piston 71 in the irrigant reservoir 1 is controlled by the ratchet mechanism which is comprised of the trigger 20, a pivot 70, about which the trigger 20 rotates, and gear 70b that engages a first ratchet wheel 77. Preferably, the ratchet mechanism is one-way such that motion of the trigger 20 toward the body handle 21 rotates the first ratchet wheel 72 that rotates to advance or contract the piston 71. In the example of FIG. 10, actuation of the trigger 20 about pivot 70a translates to rotation of the first ratchet wheel 72 via gear 70b. The rotation of the first ratchet wheel 72 is translated to the geared piston 71 and this rotation is in turn translated to a second ratchet wheel 73 that rotates in the opposite direction to the first ratchet wheel 72 that is in turn connected to a geared piston 71a in the other reservoir.

In the embodiment of FIG. 10, the device is designed to be hand-operated such that the manual actuation of the trigger 20 causes automatic motion of the two ratchet wheels 72, 73 and the geared pistons 71. The equivalent dimensions of the reservoirs 1, 4, pistons 71, 71a, and the two ratchet wheels 72, 73 shown in FIG. 10 yields an approximate 1:1 fluid exchange ratio. In addition to altering the dimensions of the aspirant 4 or irrigant 1 reservoirs, the alteration of the fluid exchange ratio can be achieved by altering the dimensions of the ratchet wheels 72, 73.

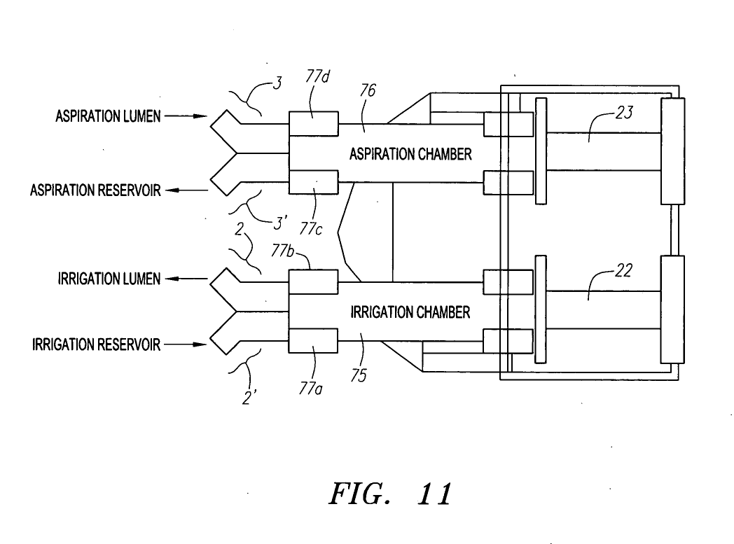

FIG. 11 shows the principles of a fluid exchange device with a segregated irrigant 75 and aspirant chambers 76 each having a dedicated inflow and outflow line. In this embodiment, the inflow line of the irrigation chamber 75 is an irrigation inflow line 2′ that communicates fluid held in the irrigation reservoir 1 to the irrigation chamber 75. The fluid is drawn into irrigation chamber 75 by the dedicated piston 22 and is subsequently expelled through the irrigation lumen 2 into the target site for fluid exchange as described previously. Similarly, fluid is drawn from the target site through the aspiration lumen 3 and into the aspiration chamber 76 by operation of the dedicated piston 23 whose motion both pulls fluid through the aspiration lumen 3 and into the aspiration chamber 76, but also expels fluid from the aspiration chamber 76 to the aspiration reservoir 3, via the aspiration reservoir outflow line 3′. This embodiment of the invention operates much like a two-stroke engine wherein fluid is pulled into the irrigation 76 and aspiration 75 chambers and subsequently expelled through the appropriate lumen. To control the flow of fluids, each of the dedicated inflow and outflow lines for each chamber have valves 77a, b, c, d that control the fluid flow. For example, when fluid is drawn into the irrigation chamber 75, a valve 77a on the chamber inflow line 2′ is opened while the piston 22 is pulled back. Subsequently, the inflow valve 77a closes and an outflow valve 77b that is in line with the irrigation lumen is opened while the irrigation chamber piston 22 is forced into the irrigation chamber 75 to expel fluids through the irrigation lumen 2. Similarly, when the action of the aspiration chamber piston 23 is used to draw out fluid into the aspiration chamber 70 via aspiration lumen 3, an inflow valve 77d on the aspiration chamber inflow line 3 is opened and the in-line valve 77b in the aspiration chamber outflow line 3′ is closed. To expel fluid from the aspiration chamber 76 through the outflow line 3′ and into the aspiration reservoir 4, the in-line valve 77d on the aspiration lumen 3 is closed and the in-line valve 77c on the aspiration reservoir outflow line 3′ is opened. As for the embodiments described above, the action of the individual pistons 22 and 23 used to cause the fluid flow throughout the system can be controlled manually by mechanical expedients affixed to the pistons. Alternatively, electronic circuitry can control the speed motion and cycle parameters of both pistons such that the fluid flow is electronically controlled according to a user interface or a predetermined fluid exchange profile. As will be apparent to one of skill in the art, the cycling action of this embodiment produces a pulsatile flow with the relative motion of both pistons 22, 23. Moreover, the particular minimum and maximum pressures in each pulsatile flow can be controlled by the relative action of the pistons 22, 23.

In another embodiment, the in-line valves are not actively controlled, but are provided as simple one-way valves that only allow fluid inflow from the irrigation 1 reservoir into the irrigation chamber 75 and, likewise only allow fluid outflow from the irrigation chamber 75 through the irrigation lumen 2. On the aspiration side of the system, one-way valves allow fluid flow only from the aspiration lumen 3 to the aspiration chamber 76, and from the chamber 76 to the aspiration reservoir 4. In use, when the device is activated, the piston plunger in either chamber will produce a positive flow through the lumen. When the lever begins to relax, the one-way valve will close and the irrigation reservoir 1 will fill the chamber. On the aspiration side, one-way valves on-both the lumen 3 and the reservoir 4 ensures that the aspirant fluid is purged into the reservoir and, during relaxation, the aspirant is extracted from the exchange site via the aspiration lumen 3. Actuation of the pistons simultaneously causes simultaneous fluid flow to and from the target site while a ½ cycle out of phase yields a transient pressure increase within the system.

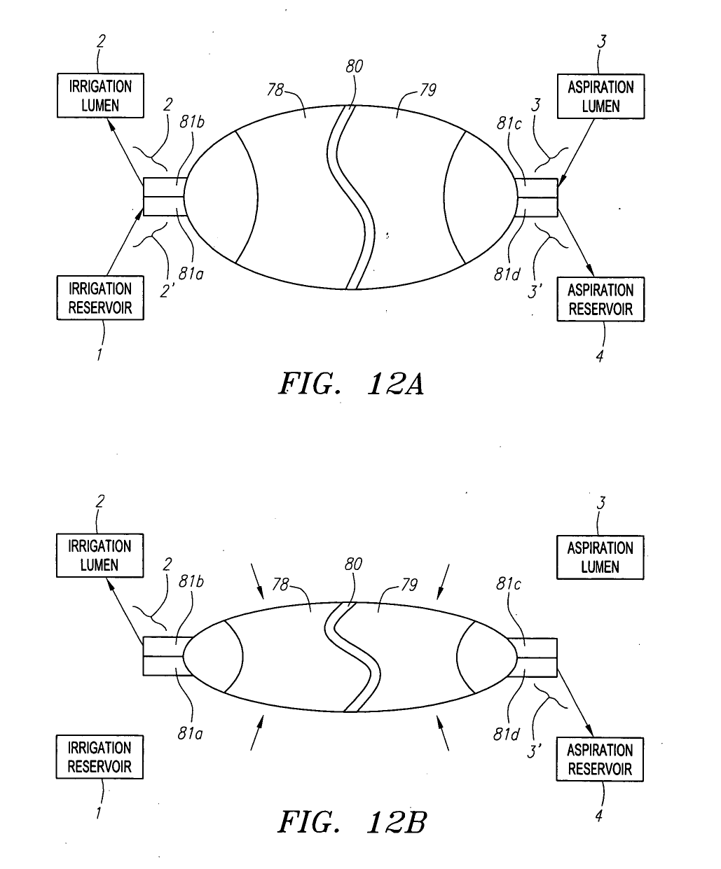

FIGS. 12A and 12B show a hand-held fluid exchange apparatus configured as a compressible handball with the internal volume divided into irrigant and aspirant aspirant chambers 78, 79 in series with dedicated inflow and outflow lines connecting irrigation 1 and aspiration 4 reservoirs, respectively. With a fluid impermeable wall disposed between the irrigant 78 and aspirant 79 chambers, the collapse of the ball under force will circulate the fluids appropriately. Referring to FIG. 12A, the apparatus is divided into an irrigation chamber 78 and an aspiration chamber 79 by a fluid impermeable barrier 80 that completely segregates the two chambers 78, 79 within the device. The expansion and contraction of the irrigant chamber 78 causes fluid flow through a dedicated inflow line 2′ between the irrigation reservoir 1 and the irrigant chamber 78 and out to the target exchange site via the irrigation lumen 2 and terminates at the target site as in the other embodiments described herein. Similarly, aspirant fluid is drawn in through the aspiration lumen 3 into the aspiration chamber 79 and out through the dedicated aspiration chamber outflow line 3′ and into the aspiration reservoir 4. As in the embodiment of FIG. 11, one-way flow valves are advantageously disposed in each inflow and outflow line between the lumen and chamber, and chamber and reservoir. Thus, a one-way flow valve 81a allows fluid flow only in the direction from the irrigation reservoir, via inflow line 2′, into the irrigation chamber 78. The fluid inside the irrigation chamber 78 may only flow in the direction through one-way valve 81b and out through the irrigation lumen 2. Aspiration fluid entering aspiration chamber 79 via aspiration lumen 3 may enter only in the direction through one-way valve 81c and aspiration fluid inside the aspiration chamber 79 may pass only in the direction of the aspiration reservoir 4 through one-way valve 81d.

Referring to FIG. 12B, pressure exerted on the compressible structure of the device, as indicated by the bold arrows in FIG. 12B, compresses both irrigant chamber 78 and aspirant chamber 79 such that fluid flows in the direction of the arrows i.e. irrigant fluid flows through one-way valve 81b, through irrigation lumen 2 and to the target exchange site. Aspirant fluid flows from the aspiration chamber 79 through the one-way valve 81d and into the aspiration reservoir 4. Fluid flow is prevented by one-way valves 81c and 81a from entering either the aspiration lumen 3 or the irrigation reservoir 1. Upon relaxation, the outer surface of the handball moves in a direction opposite to the bold arrows in FIG. 12B and the flow is reversed. Thus, fluid flows from the irrigation reservoir 1 through the one-way valve 81a and into the irrigation chamber 78. Likewise, fluid flows from the aspiration lumen 3, through one-way valve 81c, and into the aspiration chamber 79. This configuration is similar to the embodiment of FIG. 11 because a chamber 78 or 79 is provided at an intermediate position between the exchange site and the reservoir such that a volume of fluid is held at an intermediate position between each reservoir 78, 79 and the exchange site for purposes of exerting control over a discrete volume of fluid separate from the irrigation and aspiration reservoirs 1, 4.

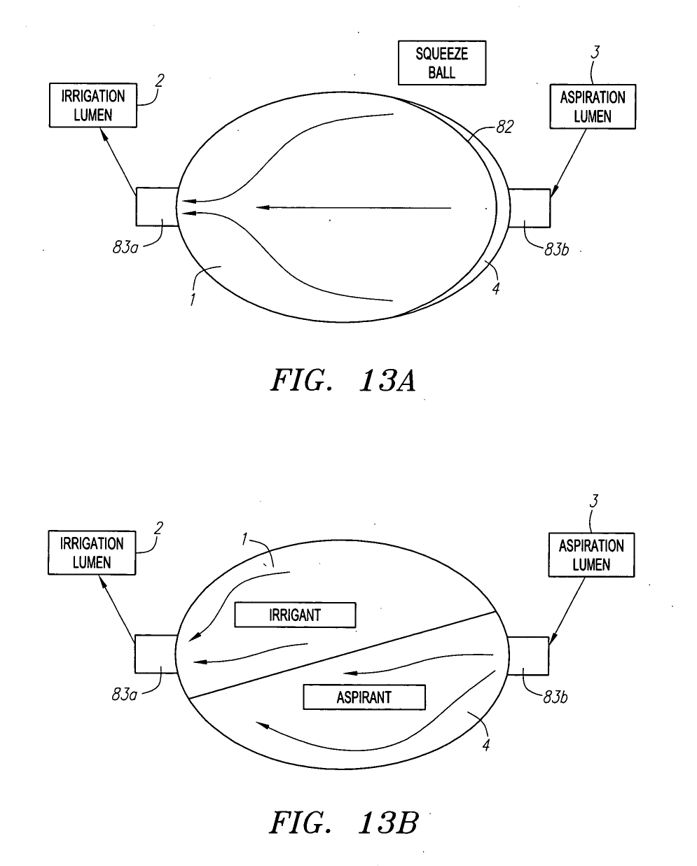

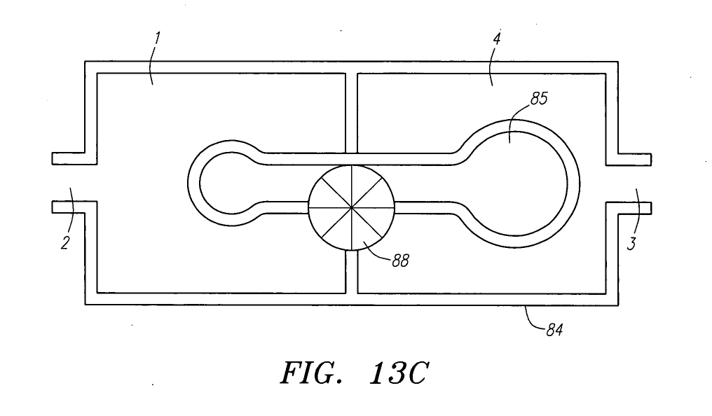

However, the compressible handball configuration can be constructed to allow direct manipulation of the irrigation reservoir 1 to expel fluid while simultaneously collecting aspirant fluid within the discrete structure of the handball itself. FIGS. 13A and 13B show a handball pump configured with an internal reservoir of irrigant and a flexible barrier 82 to separate the irrigant and aspirant reservoirs 1, 4, which are disposed inside the handball. Referring to the embodiment of FIG. 13A, prior to connection of this embodiment of the invention to a catheter element, the irrigant reservoir 1 is preferably filled with fluid to substantially encompass the entire internal volume of the handball. The flexible and fluid impermeable barrier 82 deforms towards the outer wall of the handball to accept irrigant solution and to simultaneously minimize the internal volume of the aspirant reservoir 4. When used in a clinical setting, the irrigant reservoir 1 is filled with the pharmaceutically acceptable composition to be used as the irrigant and the apparatus is sealed and may be sterilized while intact. Before using, the device is connected to the irrigation lumen 2 and aspiration lumen 3 which may be filled with fluid to establish the substantially closed loop as described previously. As in the embodiment of FIGS. 12A and 12B, one-way valves 83a, 83b are positioned in-line between the irrigant reservoir 1 and the irrigation lumen 2, and between the aspiration lumen 3 and the aspirant reservoir 4. As the handball is compressed, fluid flow generally occurs in the area of the arrows to force fluid out of the irrigant reservoir 1, through the irrigation lumen 2 and into the target site while any backflow is prevented by the one-way valve 83a. Accordingly, aspiration fluid is drawn through the aspiration lumen 3 and collects in the aspirant reservoir 4. FIG. 13B shows an embodiment of the invention wherein approximately half of the irrigant solution has been expelled through the irrigation lumen 2, exchanged at the target site, and collected back in the aspirant reservoir 4 via aspiration lumen 3. As above, fluid flow generally occurs in the direction of the arrows as the internal irrigant volume is exchanged between the irrigant reservoir 1 and the aspirant reservoir 4.

As noted above, the principal of the invention may be achieved by both user operated, generally mechanically controlled embodiments of the invention, or through electronically controlled apparatus that usually require electronically controlled pumps and/or valves. In the embodiment of FIG. 13C, a volume metric pump 86 with an internal balloon 85 is provided to achieve the fluid exchange function of the invention. Generally, the device is comprised of a housing 84 that is preferably substantially rigid and which contains an internal irrigant reservoir 1 and aspirant reservoir 4 connected to dedicated irrigation and aspiration lumens 2, 3, as described previously. Volumetric control is achieved by selectively expanding an internal balloon 85 within the housing 84 to be positioned in either the irrigant reservoir 1 or aspiration reservoir 4. As with the embodiments of FIGS. 13A and 13B, at a preliminary point in the use of the device the irrigant reservoir 1 is generally full and the internal volume balloon 85 is confined in the aspirant reservoir such that the internal volume of the balloon 85 is maximized within the aspiration reservoir4 and does not displace a substantial volume of the irrigant reservoir 1. This allows the maximum amount of irrigation fluid to exist within the irrigant reservoir 1 prior to use of the device. As the fluid exchange process occurs, the volumetric pump 86 functions by forcing a portion of the internal volume of the balloon 85 into the irrigant reservoir 1. The volumetric pump 86 may be controlled by the user or through an electrical circuitry that provides an output reading to dictate the volumes or relative percentage volumes between the reservoirs 1, 4. As the volume exchange process continues, the internal volume of the balloon 85 is transferred to a greater and greater degree from the aspirant reservoir 4 to the irrigant reservoir 1 to displace the internal volume of the irrigation fluid. At a half-way point, the internal volume of the balloon is equally disposed between the two reservoirs (assuming that the beginning volume of the two reservoirs is equal) and the volumes of the fluid contained in both the irrigant 1 and aspirant 4 reservoirs is equal. As described previously, a simple modification of the dimensions of the apparatus allow variation of the volume exchange ratio from a 1:1 value to any prescribed ratio dictated by the clinical circumstances.

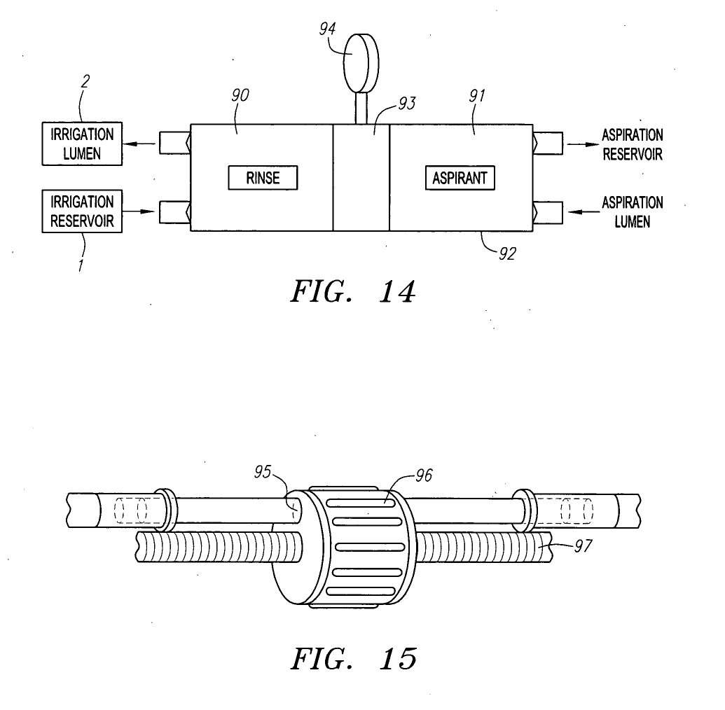

FIG. 14 shows a side view of the device where the irrigation 90 and aspiration 91 fluid impermeable chambers are contained in the same, preferably rigid housing 92 and are separated by a centrally disposed piston 93 that engages the interior of the housing 92 about the entire periphery thereof to segregate the irrigant fluid from the aspirant fluid and allows the piston 93to slide within the housing 92. By moving the piston 93 within the interior of the housing, typically from one extreme end to another, the irrigant is forced out of the irrigant chamber 90 and into the irrigation lumen 2. Fluid exchanged at the target site is collected through the aspiration lumens and into the aspirant chamber 91. Thus, in the example of FIG. 14, when the piston 93 slides from one end to the other, the irrigant chamber 90 expels irrigant, while the aspirant chamber 91 simultaneously draws in aspirant fluid. Then, as the piston 93 is moved back in the other direction, the irrigant chamber 91 refills itself with fluid from the irrigant reservoir 1 while the aspirant chamber 91 expels its contents into the aspiration reservoir 4. As in other embodiments described herein, this simple, compact arrangement allows for simultaneous irrigation and aspiration and yield a pulsatile flow. Although shown as a cylindrical housing 92, the construction and arrangement of the input, output, reservoir and piston elements could be altered without departing from the spirit of the invention. In the embodiment of FIG. 14, the piston is designed to move repeatedly and reproducibly within the housing to expel and collect a defined volume of fluid with each operation cycle.

The volume of fluid exchanged at the target site with each cycle of the piston 93 is substantially equivalent to the internal volume of the housing 92 assuming that the piston 93 is moved from one extreme to another extreme inside the housing 92 during each cycle of the operation of the device. This embodiment also demonstrates, as in the foregoing embodiments, that the fluid exchange device of the invention is readily adapted to be controlled either manually, in this case through the application of force to a handle 94 attached to the piston 93, or by electronic control, which in this embodiment would be provided by a simple pump or electrical or magnetic force to move the piston 91 within the housing 92. The separation of the irrigant and aspirant reservoirs 1, 4 from an irrigant and aspirant chamber 90, 91 permits the device to be repeatedly cycled to draw a defined volume into each chamber 90, 91 for propulsion through the irrigation lumen 2 and collection through the aspiration lumen 3. In an alternate embodiment, the entirety of the irrigant fluid to be exchanged at the target site would begin contained within an aspirant reservoir that is entirely located within the housing such that movement of the piston 91 from one extreme of the housing 92 to the other would communicate the entire volume of the irrigant reservoir 1 through the irrigation lumen 2, to the target exchange site, and back into the aspirant reservoir 4 via the aspiration lumen 3. A further example of this embodiment is shown in FIG. 15 below, having an alternate mechanical expedient for propelling fluid from the irrigant reservoir 1 into an aspirant reservoir 4.

In the embodiment of FIG. 15, the irrigant and aspirant reservoirs 1,4 are separated by a fluid impermeable barrier 95 that is movable about a threaded axis 97 or other structure that passes within a slidable member 96 that rotates and slides about the threaded axis 97 to move the barrier 95 along the axis 97 to propel the irrigant fluid. Ideally, the slidable member 96 provide for a high rate of translation, while the member 97 provides for fine travel about the threaded axis 97. The sliding element can be selectively disengaged from the threads to allow it to slide rapidly along the threaded axis for gross adjustment. When engaged, the sliding element can be rotated for fine adjustment. Interior to the sliding element is a mechanism which permits this selective thread engagement by retracting the thread contact when activated.

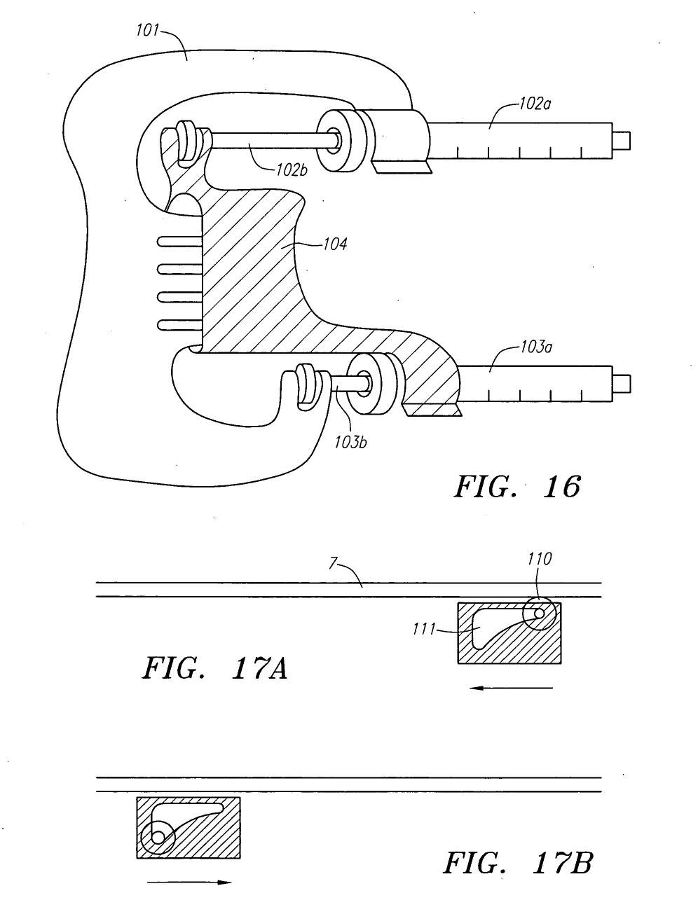

Referring to FIG. 15, this embodiment of the fluid exchange device is comprised of two main elements to achieve a configuration that allows for the body or cylinder actuation of both syringes in the desired and opposite manner. Essentially, a unitary body 101 connects of one syringe element 102a and is connected rigidly to the piston 103b of the other syringe element. A slidable element 104 engages the unitary body 101 and slides reproducibly in engagement therewith. As shown in FIG. 16, the slidable element 104 is also attached to the cylinder 103a of one syringe and the piston 102b of the other. Motion of the slidable element 104 exerts a force withdrawing one piston while advancing the other and braces the application of force by the attachment of the body 101 or element 104 to the cylinder or body of each syringe 102a, 103a. The design could incorporate existing syringes or have the syringe elements molded into the piece. There are several distinct advantages to this embodiment. One is that it ensures a 1:1 exchange ratio in terms of travel distance between the syringes. Another is that the geometric arrangement allows for a balancing of the forces involved in the device. Finally, the realization of the complex mechanics through just two moving parts is a significant advantage for the manufacturing and efficiency of the device.

As described above, the element of turbulence is important to the efficacy of the device. Since fluids tend to assimilate to laminar flow, proximity of the irrigant ports or perforations that facilitates turbulence is important for optimal rinsing of the interior of a body structure. For this reason, translation of the catheter element may accompany the irrigation or aspiration or both. All embodiments described herein can be manually translated by means of the operator's hand. Additionally, the catheter can be translated using an automated translation system similar to those used in IVUS and similar applications. Alternatively, the catheter could be translated by an element incorporated into the fluid delivery device. Referring to FIG. 17A a simple mechanism that could be used to realize this self-advancing aspect. When the catheter 7 element is moved to the left in the direction of the arrows in FIG. 17A, the round engaging element 110 slides up in the slot 111 and engages the catheter 7 to move it to the left as well.

FIG. 17B shows the same mechanism. Once the catheter element 7 is slid to the right the round engaging element 110 slides down in the slot 11 and allows the catheter element 7 to slide freely to the right in the direction of the arrow without interacting or affecting the catheter's position. This allows for the selective retraction or advancement of the catheter 7 by a predetermined amount with each squeeze of the device. There are many ways in which this element could be realized. The simplest would be an apparatus that selectively grasps the catheter when moving one direction and idles or does not grasp when moving in the opposite direction. A guiding track that biases the element could be used to apply pressure and grasp the catheter moving in one direction and then release and allow idle sliding to the reset position in the other direction. This element could be selectively engaged by the operator when needed, and could be developed to allow for selection between advancement and retraction of the catheter.

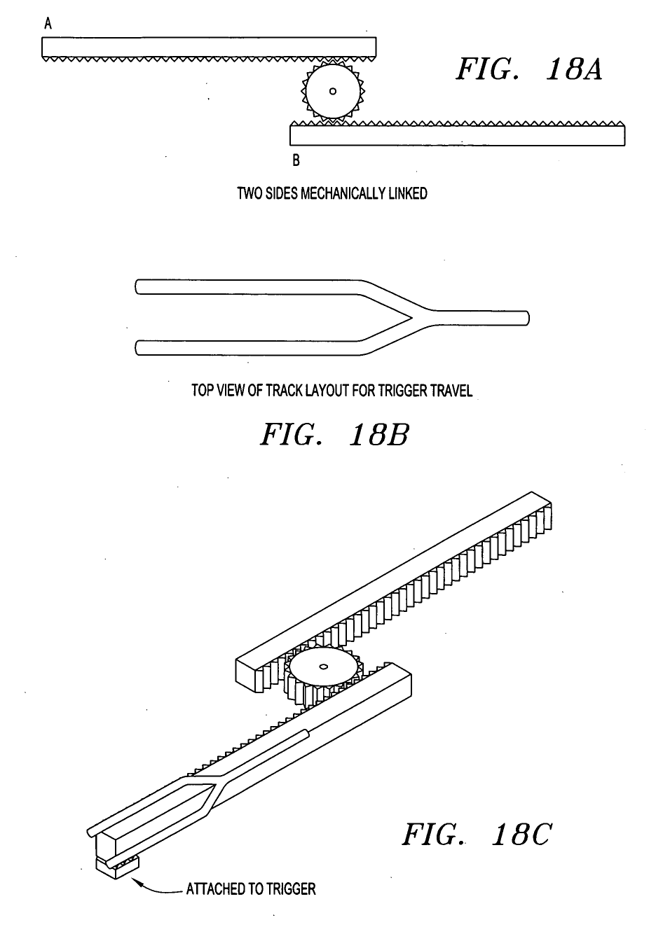

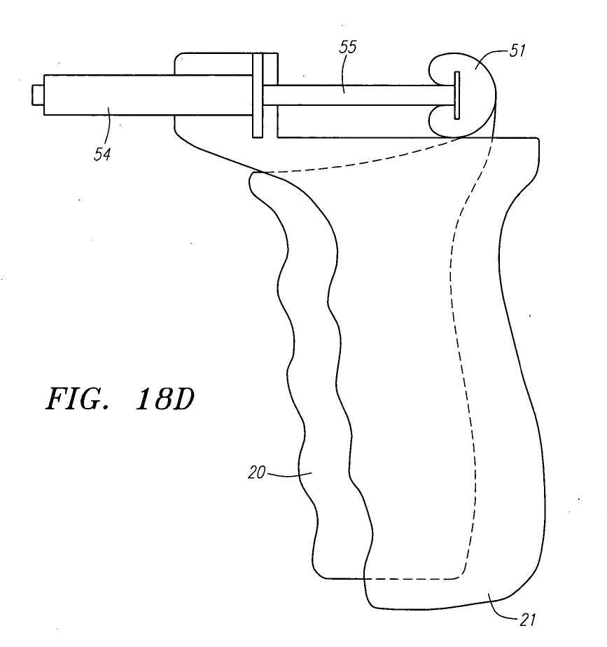

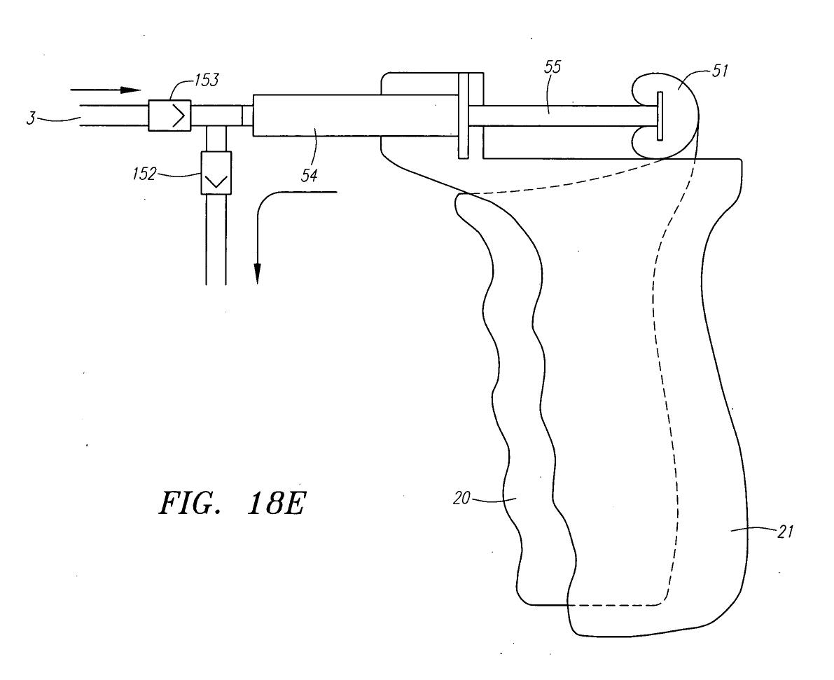

In the present preferred embodiment of the fluid exchange device, it is necessary to have a reset force supplied by an element such as a spring inherent in the device. This reset force is added to the resistance in the system that must be overcome by the operator to utilize the device. In some cases, an embodiment where this force was minimized or eliminated would allow more of the force generated by the operator to be directed to the work the device is performing and not to overcoming the reset force element. Referring to FIGS. 18A-18C, this function could be achieved through the use of a staged device. FIG. 18A shows a simple mechanical way in which the two sides of the device could be linked mechanically. It is important in this embodiment that the two sides be linked mechanically so that they behave in an equal and opposite manner. This is necessary so that the trigger can be actuated repeatedly in the same manner but engage just one of the sides while still driving the entire system. This allows the benefit of having the operator not realize the changes occurring internally in the device. The squeezes would not feel substantially different. In this embodiment, the first squeeze would activate the two chambers and the second squeeze would reset the two chambers. A simple mechanical setup could achieve this result. Similar mechanisms are commonly used in objects such as retractable ball point pens. Essentially, an element attached to the trigger element would be slightly biased to selectively engage one side or the other of the device. FIG. 18B shows a top view of the track layout that would guide the selectively engaging element of the trigger. With the two sides linked mechanically to travel in equivalent and opposite manners as described elsewhere, the force of the trigger element could always be applied in the same manner with varying effect. With the aid of the minimal return force element, the trigger is brought back to its full and extended position and biased to one side so that it will slip into the opposite track for the next actuation of the trigger. After that actuation, as the trigger is returning to its default position, it will be biased to one side of the device and slip easily into the track of the opposite side.

FIG. 18C is a diagram of how the system could be achieved such that each time the trigger is expanded, it engages the other side of the device and pulls it back when squeezed.

Referring to FIGS. 18D and 18E, there may be circumstances which render desirable the opportunity to operate either the irrigation or the aspiration side of the device independently. Since the operation of the device withdraws aspiration fluid by a predetermined amount for each actuation or cycle of operation, and infuses irrigation fluid by a predetermined amount for each actuation or cycle of operation, each “side” of the device, either the irrigation or aspiration side may be operated independently. In such cases, the non-elected function is simply disconnected. This could be achieved easily with the present embodiments described herein by removing or disengaging one of the syringe elements. Alternatively, an embodiment of the device could be constructed to isolate a single function. This could be a device that simply infuses fluid in a stepwise fashion, or one that'simply aspirates fluid in a stepwise fashion. In a preferred embodiment of this aspect of the invention, an aspiration only device is provided. The utility of this device improves upon a simple aspirating syringe or other vacuum-creating apparatus because the amount of vacuum created in an aspiration lumen accumulates in a stepwise fashion and is cumulative with each operative cycle of the device. A limitation of straight aspiration with a simple, non-valve enhanced, syringe is that the vacuum created by pulling on the syringe is greatest when the plunger is first pulled. As the syringe fills with aspirate, the strength of the vacuum aspiration pull degrades. With the invention herein described, the valved system allows for superior aspiration, because with each actuation of the device, the vacuum in the system is increased. In the example of a large, organized thrombus at the tip of the aspiration lumen 3, straight aspiration will simply pull a given vacuum pressure that quickly degrades over time. Stepwise, or valve-assisted aspiration as described herein will pull a greater and greater vacuum with each actuation of the device. This use pattern results in more advantageous aspiration.

The device includes at least one in-line valve in the aspiration lumen 3 and preferably includes a second one-way valve in the fluid line that transfers aspirant fluid to the aspiration reservoir 4 or waste. Referring to FIGS. 18D and 18E, the fluid exchange device is comprised of a simple syringe 54, 55 removably attached to a hand-held actuation of the type shown in FIG. 4B and accompanying text, but modified as described here. Actuating the device by squeezing the trigger 20, retracts the syringe 55 and creates a vacuum in the aspiration lumen 3. Fluid is drawn back through the aspiration lumen 3 in a quantity proportional to the distance withdrawn on the plunger 55 of the syringe and the internal bore 54 of the syringe. Upon release of the trigger 20, a constant force bias, such as a spring (not shown) or other stored energy source, causes the syringe to move back to its original position at the beginning of the cycle, while the one-way valve 152 in line with the aspiration lumen 3 prevents the back flow of fluid towards the patient. At the same time, the fluid pressure created by the constant force bias forces the aspirated fluid down the aspiration lumen 3 and through the second one-way valve 153. In this manner, a stepwise aspiration can be repeated as many times as desired with the advantage that the vacuum force created on the aspiration fluid is controlled, additive, and increases with each actuation of the device. The mechanical expedients by which the device operates to create the vacuum pull can be achieved in several different configurations. In one embodiment, a spring force can be applied to return the handle and trigger to the original or default positions. The force to exert the fluid during the first cycle of the operation is provided by the user. The actuating force applied to the syringe 54, 55 can be provided by the gear mechanism disclosed above in FIG. 18A and the accompanying text in a lever or pulley and drive cable configuration as disclosed above and FIG. 8 and accompanying text or with a conventional hydraulic system where actuation of the handle of the device creates a fluid pressure that withdraws the plunger 55 of the syringe to create a vacuum in the aspiration lumen 3.

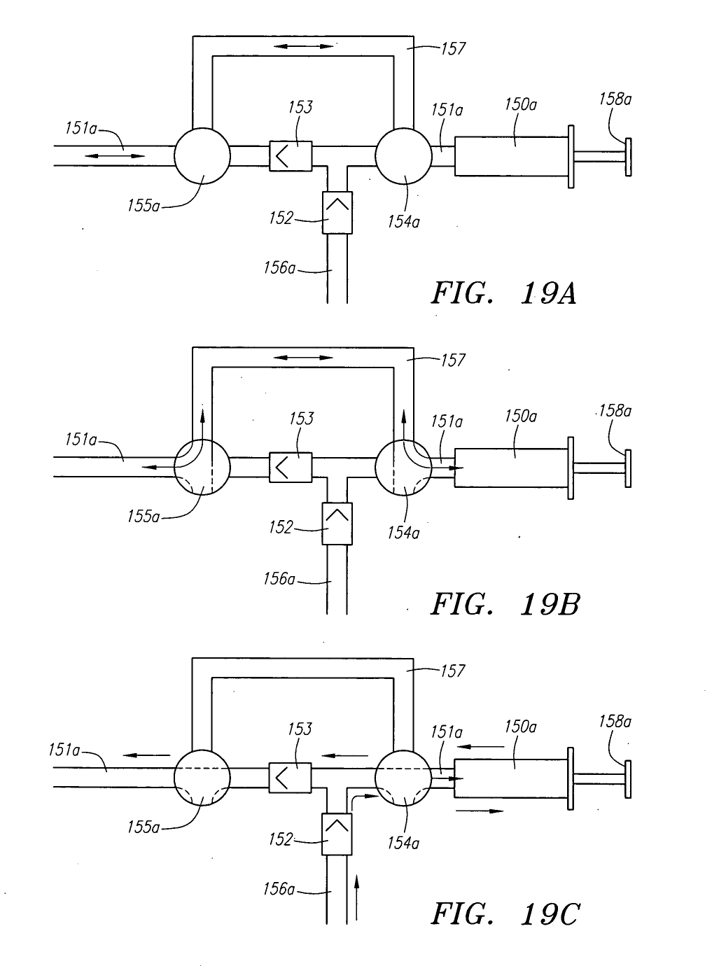

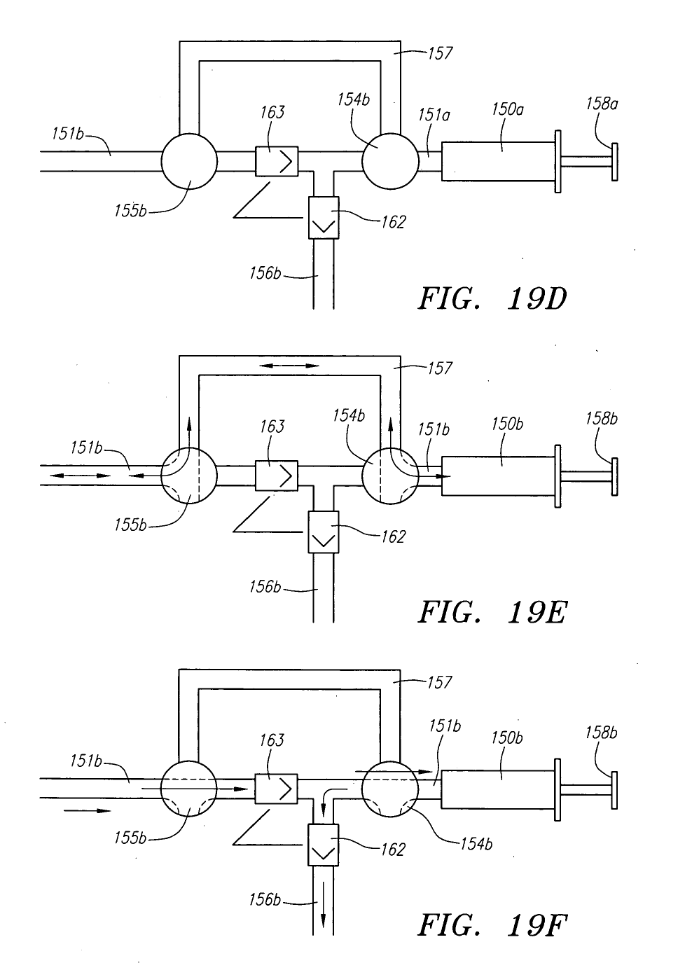

FIGS. 19A through 19F are an embodiment of the invention that permits a closed circuit recirculation of small fluid volumes through a treatment site without large volume fluid exchange. As noted above, certain clinical indications benefit from the recirculation of a small volume of fluid, usually containing an active, high value pharmaceutical, through a treatment site. For this purpose, the irrigation and aspiration fluid pathways may be altered to establish a smaller volume fluid circuit wherein fluid is recirculated to concentrate a portion of the fluid at the treatment site. This configuration adds a bypass loop and additional three-way valves on both the irrigation and aspiration side of the fluid exchange system. Referring to FIG. 19A, the closed fluid circuit is provided on the irrigation side by an irrigation fluid chamber 150a which may comprise any fluid reserve component as described herein. In the embodiment of FIG. 19A, the irrigation chamber 150a comprises a simple syringe 150a with a plunger 158a in fluid communication with a branched irrigation lumen 151a having two conventional one-way valves 152, 153 and two conventional rotating three-way valves 154a, 155a. In this configuration, a first one-way valve 152 is located in a branch of the lumen 151a and between the irrigation fluid reservoir (not shown), the lumen 151a and the remainder of the fluid circuit. A second one-way valve 153 is located between the three-way valves 154a, 155a. On the irrigation side, this first one-way valve 152 permits flow only in the direction from the irrigation reservoir, through the lumen side branch 156a and to the remainder of the system. The second one-way valve 153 permits flow only down the branched lumen 151a, between the three-way valves 154a, 155a, and down the irrigation lumen to the patient. The two three-way valves 154a, 155a operate to direct fluid flow into and out of the bypass loop 157 and are disposed in-line of the branched lumen 151a.

Referring to FIG. 19B, the recirculation mode on the irrigation side is achieved by rotating three-way valves 154a, 155a to direct fluid flow through the bypass loop 157 by rotating the valves to bypass the second one-way valve 153. Referring to FIG. 19C, following the use of the recirculation mode, the system may be returned to fluid exchange mode by rotating the three-way valves 154a, 155a to produce fluid flow along the path of branch lumen 151a while avoiding the bypass loop 157.

Referring to FIG. 19D, the aspiration side of the system is essentially the same as the irrigation side in a mirror image, with the syringe 150b operating in the opposite direction. However, a third one-way valve 162 permits flow only in the direction from the branched lumen 151b, through lumen side branch 156b, to the aspiration reservoir (not shown). The fourth one-way valve 163 is located between the two three-way valves 154b, 155b and permits flow only in the direction from the patient to the aspiration reservoir or syringe 150b.

On the aspiration side, the recirculation mode is achieved in the same manner as on the irrigation side, namely, the three-way valves 154a and 154b are rotated to direct fluid flow through the bypass loop 157. Fluid flow is shunted around the third and fourth one-way valves 162, 163 and into the aspiration chamber of syringe 150b which is in fluid communication with the branched lumen 151a. Again, as with the irrigation side, the fluid exchange mode is re-established by simply rotating the three-way valves to avoid the bypass loop 157 and to direct fluid through the fourth one-way valve 163.

Referring again to FIGS. 19C and 19F, in ordinary fluid exchange operation, with the three-way valves rotated to avoid the bypass loop 157, the fluid drawn from the irrigation reservoir passes up the branch of branch lumens 151a, 151b and into the chambers comprising the syringes 150a, 150b upon actuating the plungers 158a, b. Advancing the plunger causes fluid to pass through the second one-way valve 153 and through the branched lumen 151a, down the infusion lumen of the catheter to the treatment site. Simultaneously, referring to FIG. 19F, the fluid is drawn through the aspiration lumen and into the branched lumen 151a, through the fourth one-way valve 163 and into the aspiration chamber comprising the syringe 150b. In the context of recirculation of a small volume of a therapeutic agent, this embodiment operates by introducing the therapeutic agent into the infusion side of the system, typically from the irrigation reservoir 1 or the syringe to permit circulation within the bypass loop 157.

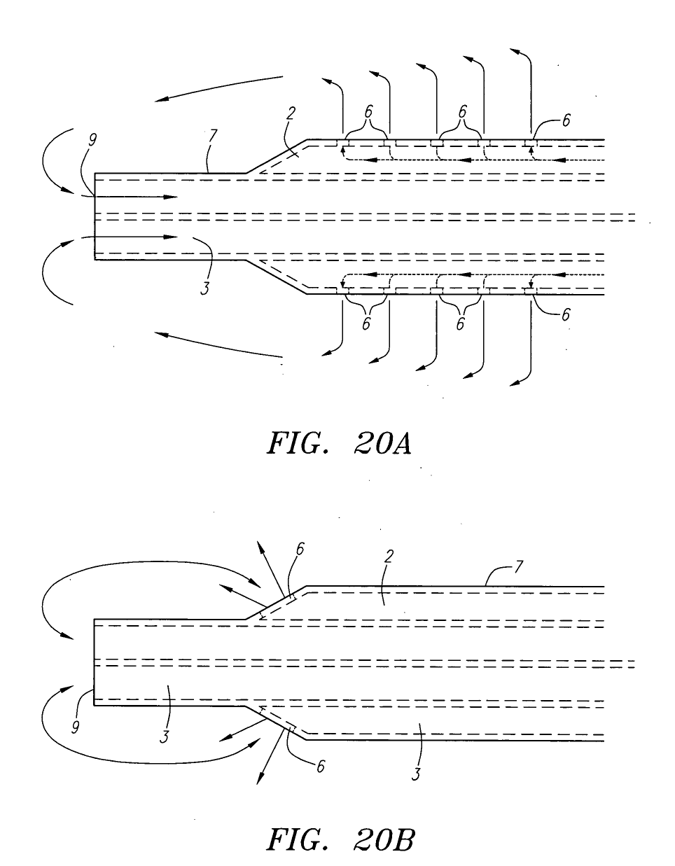

As noted above in connection with the discussion of the embodiment of FIG. 20, the fluid exchange device of the invention can be used either to irrigate or aspirate fluids independent of fluid exchange. Accordingly, several different designs for the distal portion of the catheter element 7 are desired. This distal portion, sometimes referred to as a rinse tip or rinse nozzle, is designed to provide desired fluid flow and turbulence parameters depending on the clinical indication. Although these designs are described principally as providing a plurality of irrigation ports 6 in varying geometries, as described above, the ports could serve as aspiration ports by simply reversing the dedicated lumens. Also, as described above in FIG. 20, the irrigation ports 6 could be oriented distal of the aspiration port(s) 7 or vice versa. Accordingly, these openings are simply termed “ports” 200 to indicate dual function.

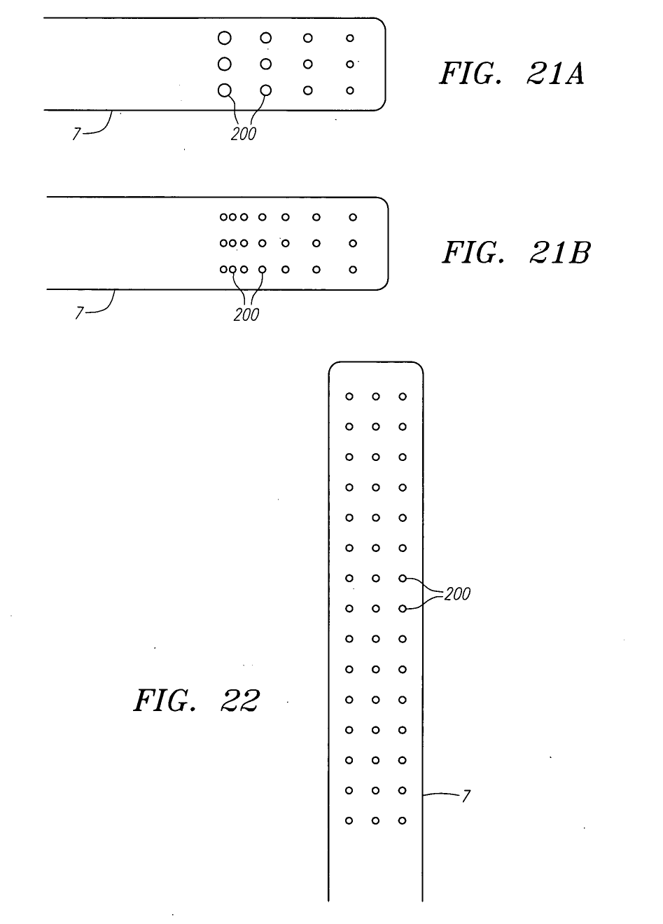

FIG. 21a shows a series of ports 200 in the distal tip of the catheter 7 which vary in size. The size of the ports 200 increase toward the proximal end of the catheter 7. This arrangement allows for fluid to exit the catheter 7 at a greater volume and lower velocity at the proximal end of the rinsing region. The inverse arrangement, with the ports 20 increasing in size toward the distal tip of the catheter 7 is also contemplated. Such an arrangement would allow for a greater volume, lower velocity of fluid to leave at the distal end of the catheter 7. Other possible arrangements of variations in port 200 size could include holes increasing in size as one approaches the midpoint of the rinse region. Such variations are easy to envision and each offers a slightly differing and therefor advantageous flow result.

FIG. 21b shows an arrangement of ports 200 that increase in density toward the proximal end of the rinsing region. The denser region will have more fluid ejecting. Likewise, these ports 200 could be arranged such that the increased density was at the distal tip of the catheter 7. Additionally, the density of ports 200 could be arranged to be centered or nearly centered in the rinsing region. The density could also be varied in a circumferential instead of longitudinal manner.

FIG. 22 shows an arrangement of ports 200 illustrating the potential for holes creating an extended region of rinse. Such an arrangement allows for more generalized, diffuse fluid introduction. Such flows could be less traumatic and more gentle on the vessel. In contrast, there may exist circumstances where a more localized, specific flow is desired. For such instances, fewer ports of defined geometries as detailed elsewhere in the drawings would be useful.

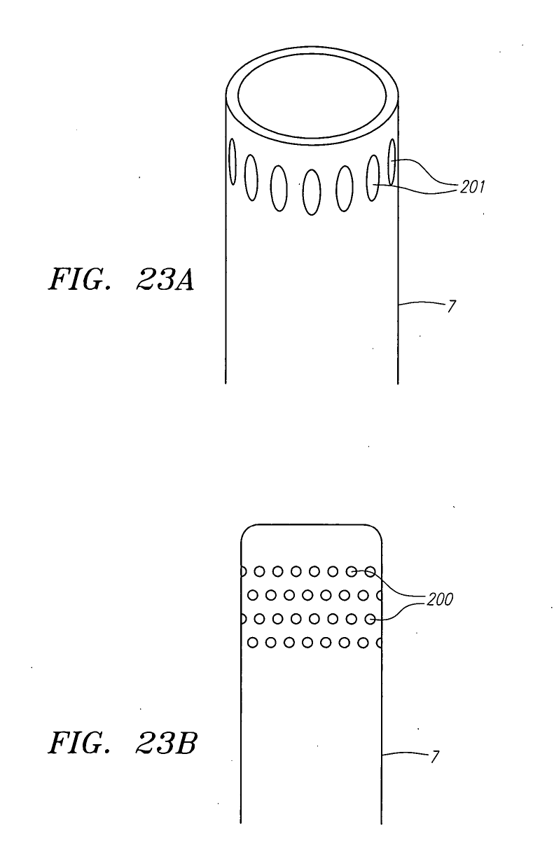

FIG. 23A shows longitudinally oriented ports 201. It may be that such a geometry is found to be advantageous for fluid introduction. FIG. 23B shows a multitude of closely set ports 200. Such an arrangement allows for a larger volume of fluid to be transported to or from a more specific, localized region.

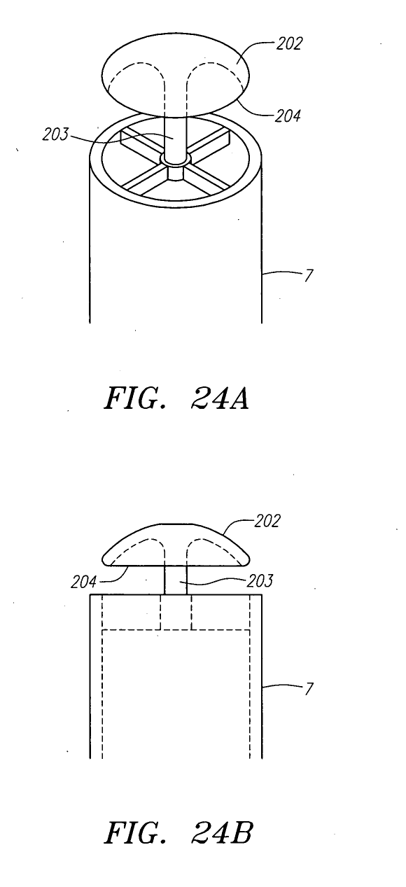

FIG. 24 shows an extension piece 202 adapted for insertion into the distal tip of the catheter 7 to effect a reversal of flow back in the proximal direction. The extension piece 202 attached or comolded to the distal tip of the catheter 7 may have a post 203 and a deflection disk 204 to redirect the fluid flow. This configuration offers a large area for the ejection of fluid, thereby reducing the flow resistance within the lumen. Such a configuration could allow for a smaller lumen catheter to be used to achieve flow levels similar to those of larger catheters with smaller areas of fluid ejection.

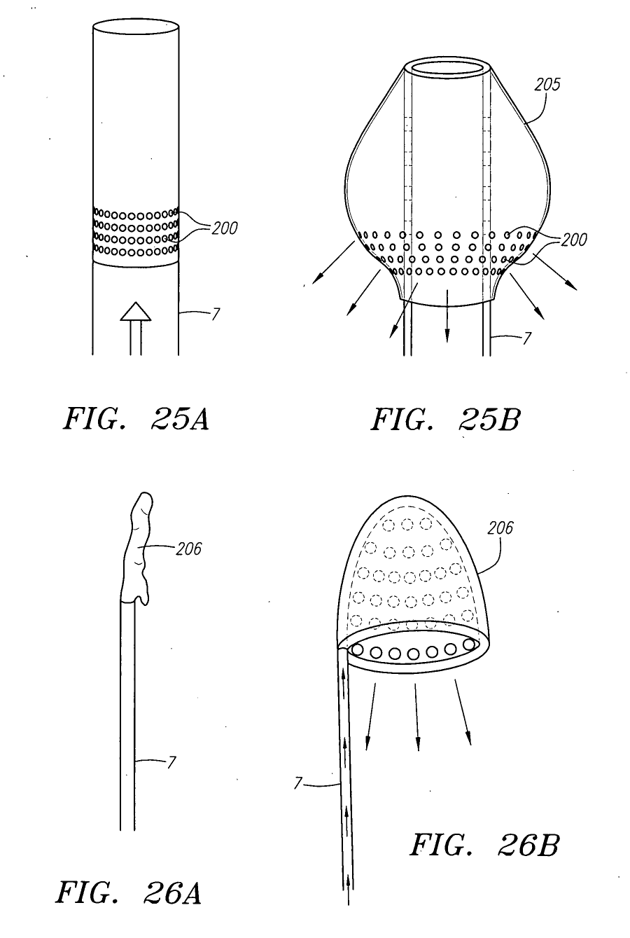

Given advances in balloon and similar technologies, FIG. 25 may offer a feasible arrangement for achieving the goal of directing fluid in the proximal direction. FIG. 25A shows the distal tip in its deflated mode for introduction to and removal from the site of interest. FIG. 25B shows how the fluid sent down the irrigation lumen 2 of the catheter 7 would fill the balloon or occlusive element 205 and be expelled in the desired direction dictated by the placement and geometry of the ports 200. Here it is shown ejecting fluid in the proximal direction. The rinse element could be constructed of balloon like materials such as latex, polyurethane or other such polymers. The material could be of either a compliant or a non-compliant nature. Care in construction would be taken for the creation of the holes in a manner such that they are not-vulnerable to tearing or enlarging in an undesired manner.

FIG. 26 shows another embodiment which allows for a minimal profile for insertion and removal. An expandable shell 206 deploys under the force of the fluid being introduced. Due to the geometric arrangement of the ports 200 on the inside of the created shell 206, the fluid flow would be directed in the proximal direction. The deployable shell device 206 could be realized using materials such as those used in medical balloons: latex, polyurethane, silicone, and others.

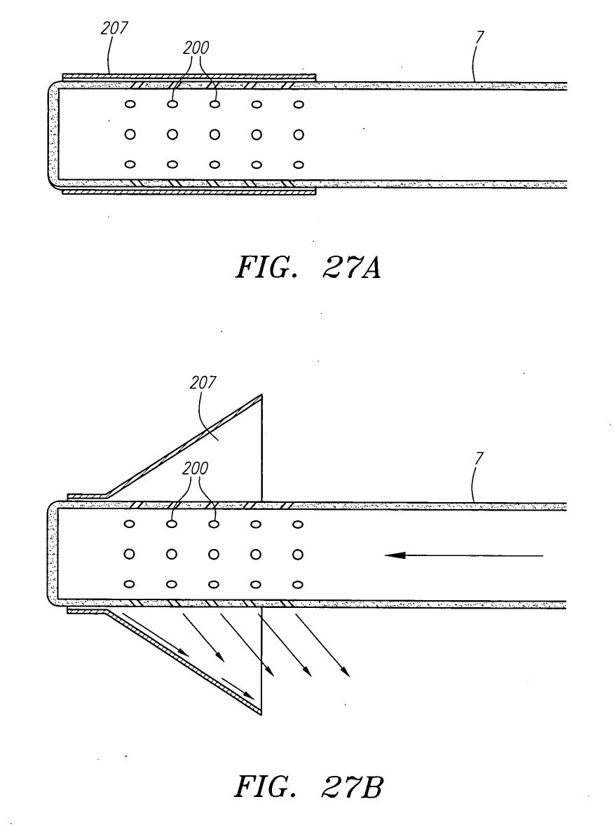

FIG. 27 illustrates a configuration of the distal region of the catheter 7 achieved by placing a piece of material as a cover flap 207 over the ports 200. FIG. 27A shows the distal tip of the catheter 7 with the flap 207 in the default and undeployed configuration for ease of insertion and removal. When fluid pressure is applied to the catheter 7 the fluid being ejected through the ports 200 forces the flap 207 away from the catheter 7, creating a structure that directs fluid flow in the desired direction (here the proximal). FIG. 27B shows the fluid exiting the catheter 7 and holding the flap 207 structure open. The flap 207 could be constructed of an elastic material that retained its shape, or a non-elastic material with similar tendencies.

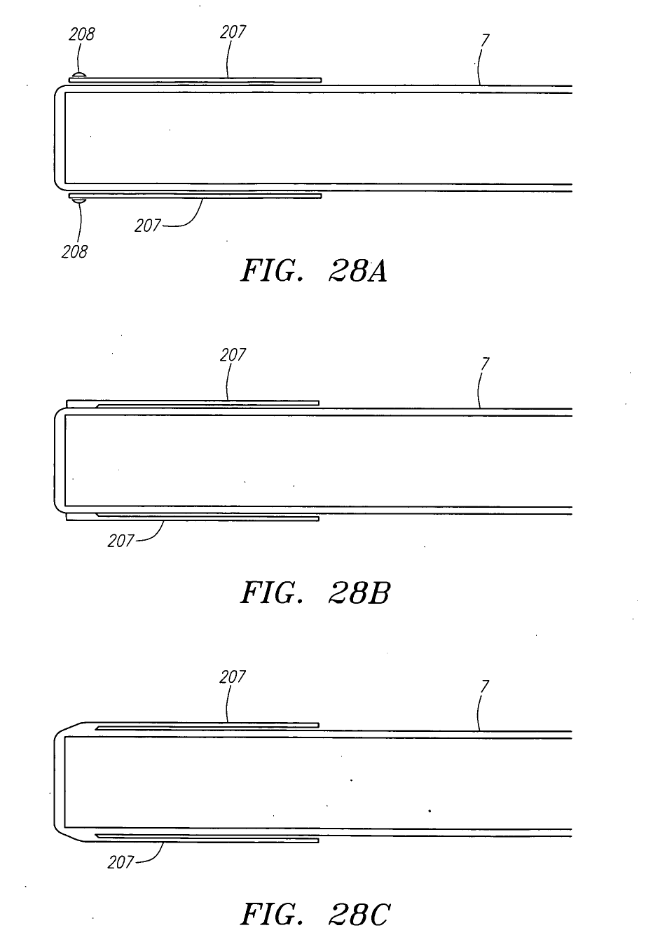

FIG. 28 shows a few construction techniques for attaching such a flap 207 to the catheter 7. FIG. 28A shows a small band 208 that could be used to hold the flap 207 to the catheter 7. FIG. 28B shows the flap 207 attached by adhesive or heat welding. The flap 207 could also be co-molded with the tip as shown in FIG. 28C. Other similar techniques for attachment include variations of heat bonding, adhesives, molding, and other commonly utilized processes in medical device manufacturing.

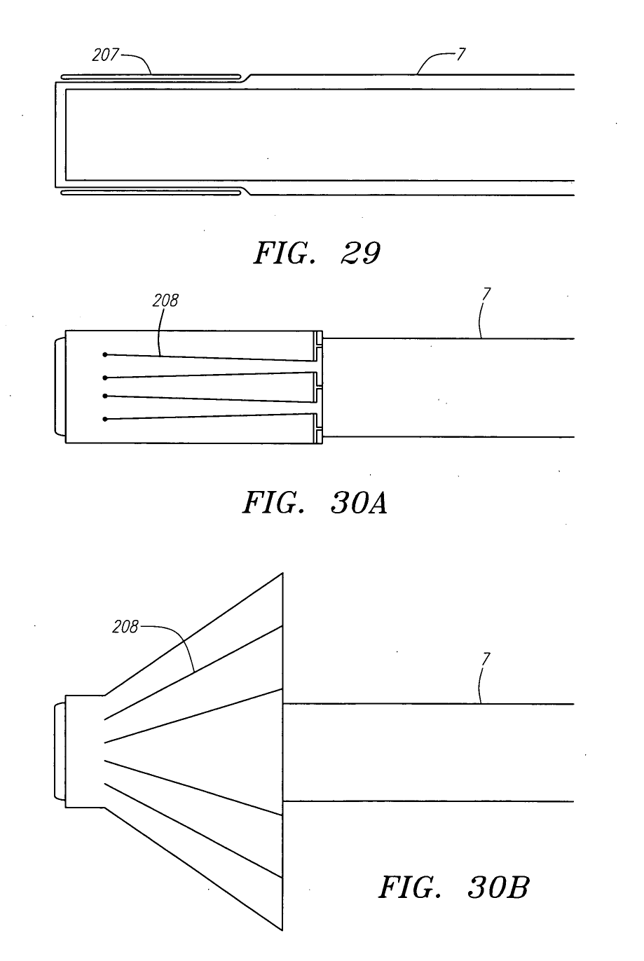

FIG. 29 shows a detail of how such a flap 207 could be inset into the catheter 7. This could be realized so that the flap 207 was either flush with the catheter 7, or slightly recessed, as desired. Such an arrangement could assist in securing the flap 207 in place for insertion and especially removal.

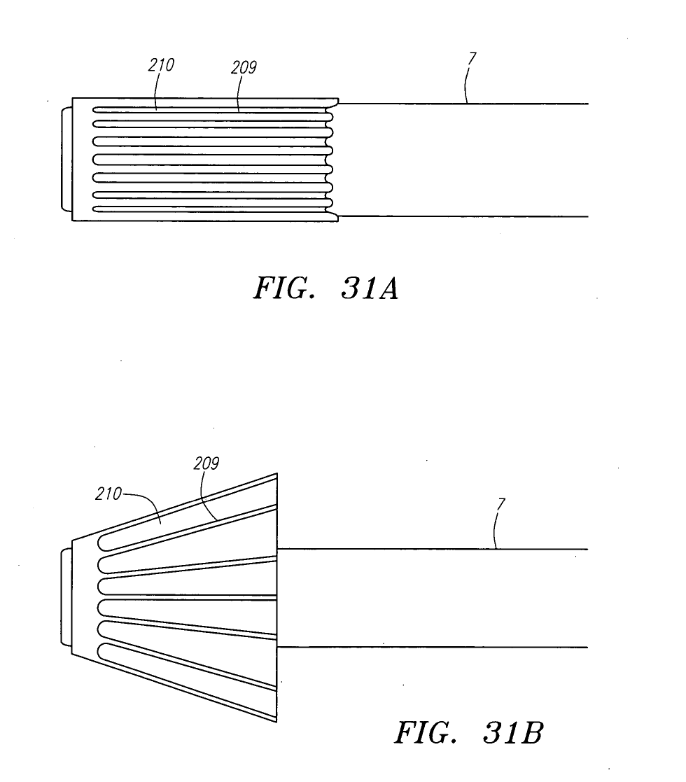

FIG. 30 shows an arrangement which would allow for the expansion and collapse of the flap material 207 through the use of pleats 208. In this embodiment, the flap 207 would be collapsed for insertion and removal as in FIG. 30A. Then, the flap 207 could be deployed by fluid or other means to resemble FIG. 30B. Another manner of achieving similar results is illustrated in FIGS. 31A and 31B. Here structural ribs 209 are used to support a flexible, elastic webbing material. FIG. 31A shows the low profile configuration for insertion/removal.

FIG. 31B shows the device deployed with the ribs 209 supporting the webbing material 210 and thereby creating a structure to direct fluid in the desired manner.

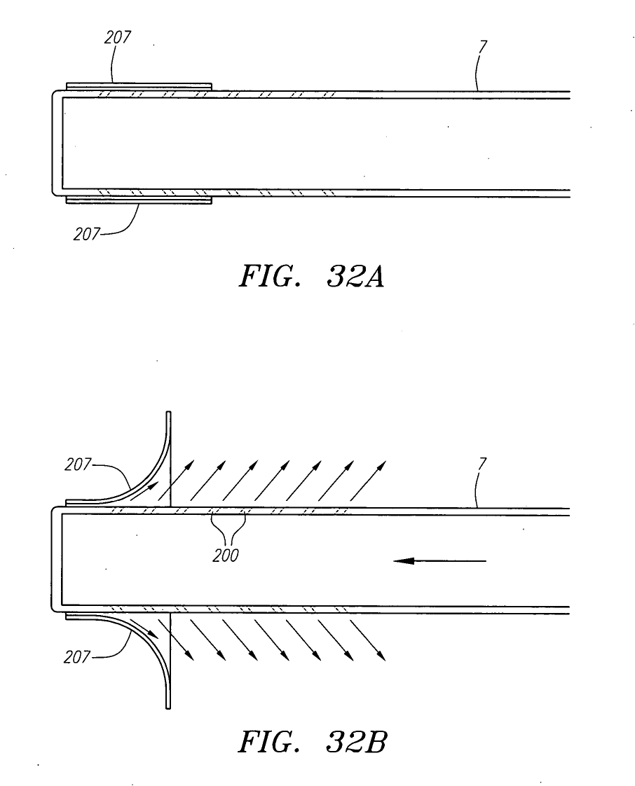

Full coverage of the ports by the flap 207 may not be necessary. FIG. 32 illustrates such a case where the flap 207 covers only a portion of the ports 200, which may be enough to create the desired fluid flows. FIG. 32A shows such an arrangement collapsed for easy insertion/removal. FIG. 32B shows the fluid exiting such an arrangement in the desired manner.

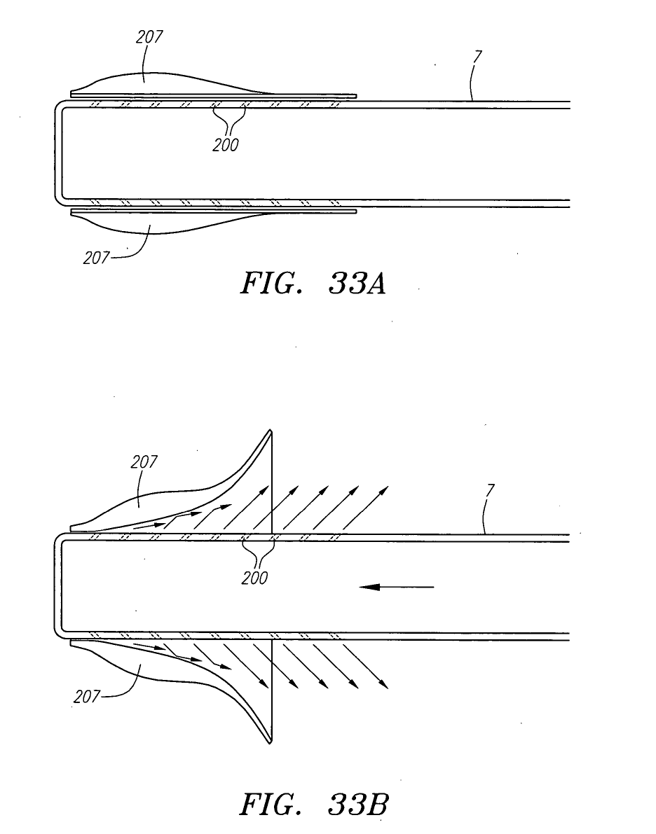

It may be desired to regulate the manner in which the cover flap 207 deforms under the pressure of the fluid being introduced. One such way of regulation is illustrated in FIG. 33. Variable deformation of the flap 207 can be achieved by varying the thickness of the flap 207 material. Where the material is thicker, the flap 207 will be more resistant to deformation.

This allows the outermost edge of the flap 207 to be made of very thin material that is very atraumatic to the vessel interior. It also allows the thicker region to provide a force on the flap 207 to cause it to regain its original, default shape. FIG. 33A shows the tip of the catheter 7 ready for low profile insertion/removal. FIG. 33B shows the flap 207 deployed by the fluid, with the varying thickness producing a non-regular shape of the deployed flap 207.

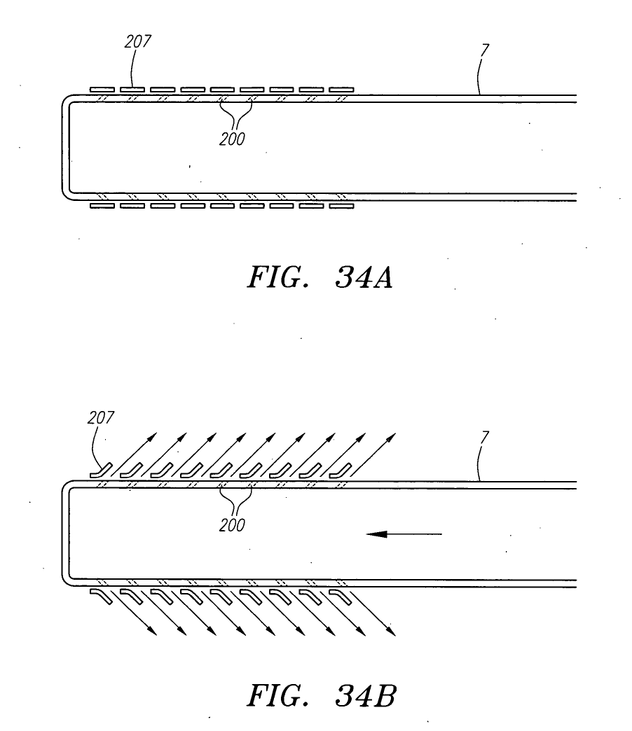

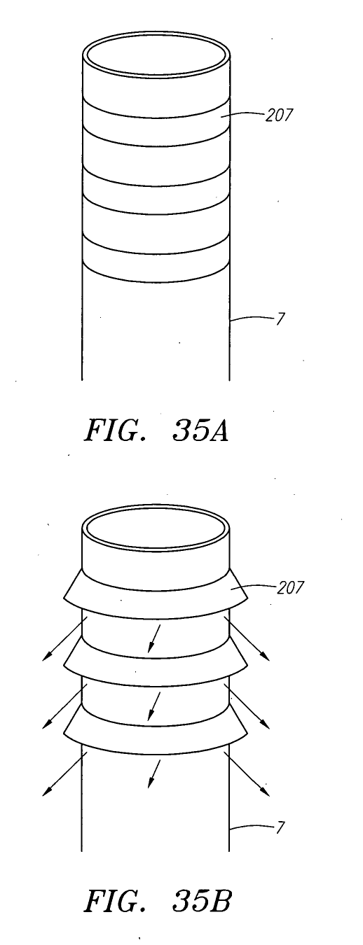

Optimal fluid flows may be achieved by placing a flap 207 or shield over each port 200 or layer of ports 200 as in FIG. 34. FIG. 34A shows the collapsed configuration offering a low profile for insertion/removal. FIG. 34B shows fluid ejecting from the ports 200, directed by the flaps 207. Such an arrangement allows for more direct interaction between the fluid and the directional flaps 207, which may achieve the desired fluid flow more effectively. This arrangement also limits the degree to which the flap 207 extends away from the catheter and may interact with the vessel wall. One such arrangement could be achieved with circumferential flaps 207 like those pictured in FIG. 35. FIG. 35A shows the collapsed form for insertion/removal. FIG. 35B shows the flaps 207 distended by the ejecting fluid.

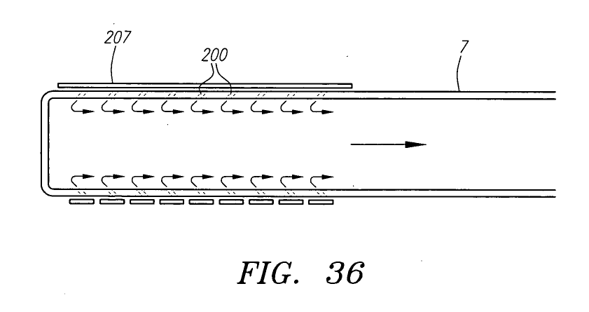

Another potential advantage of such a construction with exterior flap(s) 207 covering the ejection port(s) 200 is that a slight suction applied to the interior of the catheter lumen would serve to secure the flaps 207 to the catheter for removal. This simple arrangement would insure that the flaps 207 stay in the desired position as illustrated in FIG. 36.

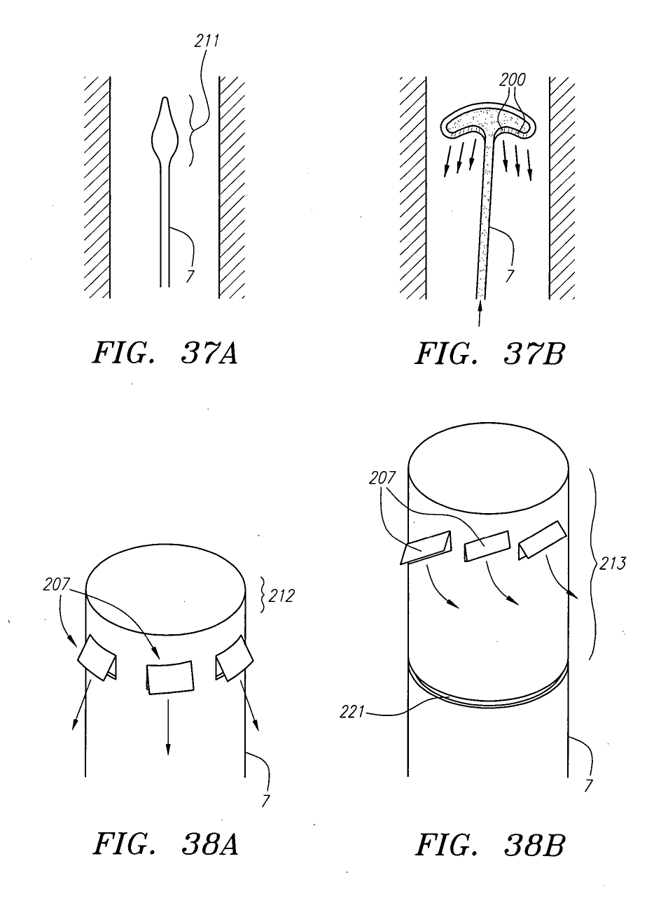

FIG. 37 shows a conformable distal tip 211 for the introduction of fluid into a vessel. FIG. 37A is the conformable distal tip 211 in the undeployed, low profile form for insertion and removal. FIG. 37B shows the tip 211 deployed and showering fluid proximally. The force of the fluid would expand the tip 211 and create the geometry which then expels the fluid in the proximal direction according to the arrangement of the ports 200.

FIG. 38 offers a construction that would enable the directing of fluid flow utilizing simple flaps 207 over the ports 200. FIG. 38A shows such flaps 207 pushed outward by the ejecting fluid. The flaps 207 direct the fluid proximally. FIG. 38B has similar flaps 207 except that they are angled to provide fluid flow in a circular manner. This could be achieved in two distinct manners. In one, the catheter tip 212 would be secured by being part of a 1:1 torqueable catheter and the fluid would flow around it in a circular pattern. Alternatively, the distal end of the catheter is a rotatable tip 213 powered by the rotational motion of the ejecting fluid.

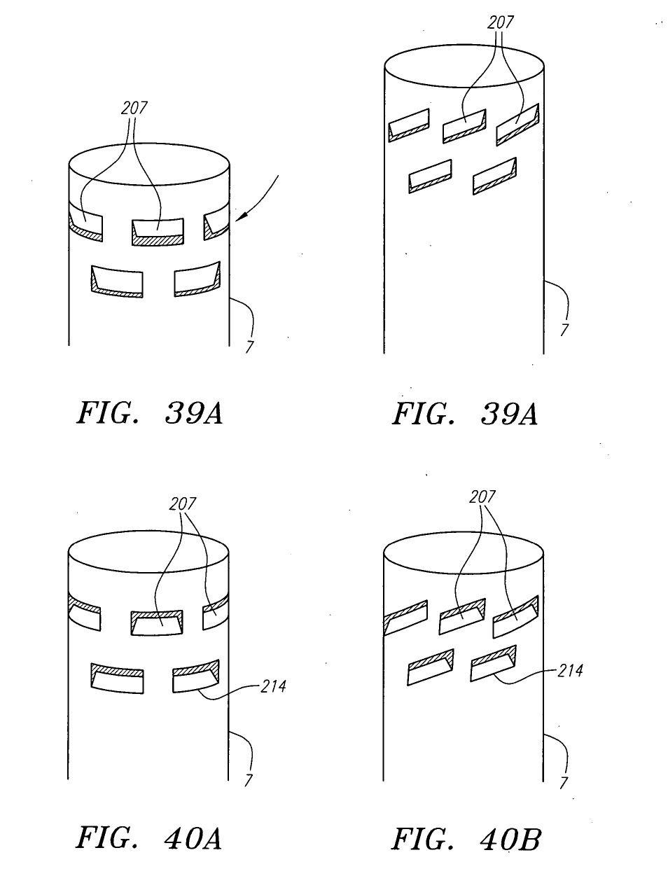

FIG. 39 shows a similar setup with the flaps 207 to the inside of the catheter. This gives the advantage of eliminating the possibility that the flaps 207 come into contact with the vessel walls. FIG. 39A shows the standard horizontal arrangement. FIG. 39B has the flaps 207 angled to produce the rotation of fluid as discussed previously.

FIG. 40 has the flaps 207 folded to the inside as well, except in this arrangement that are attached at the bottom, or proximal side 214. This configuration allows the internal flaps 207 to direct the fluid flow in the proximal direction. FIG. 40A shows the horizontal configuration while FIG. 40B shows the angled arrangement.

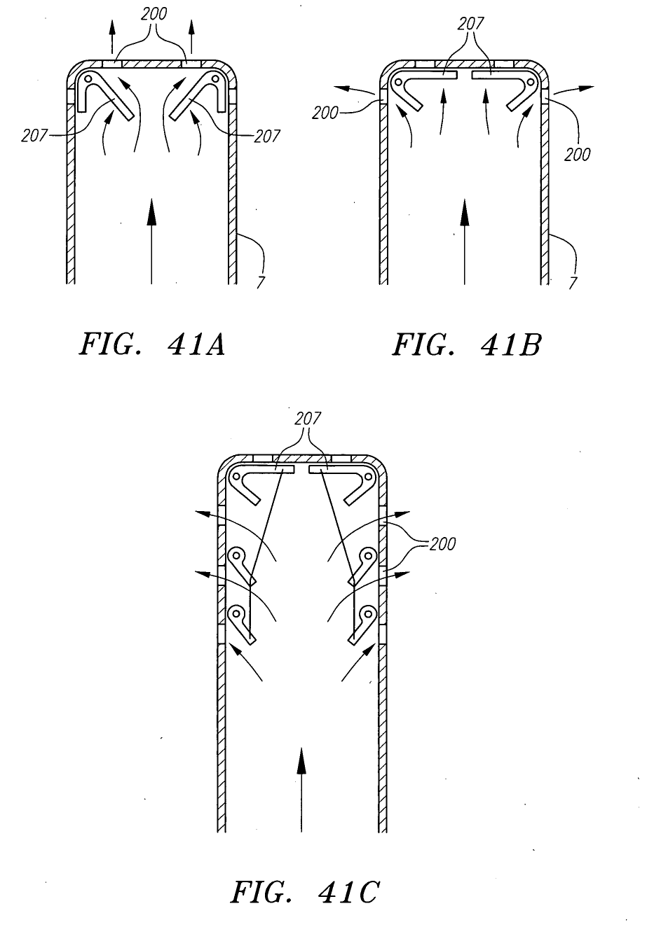

FIG. 41 shows a mechanism by which the internal flaps 207 could be regulated to selectively open and close if desired. The ejecting fluid itself is used to activate the system in this arrangement. FIG. 41A shows the default position of the flaps 207. The fluid progressing out the end of the catheter 7 would push the levers, thereby sealing the ports 200 at the end of the catheter 7 and opening those on the sides, as shown in FIG. 41B. FIG. 41C shows one way in which such a system could be linked to provide similar function to multiple ports 200.

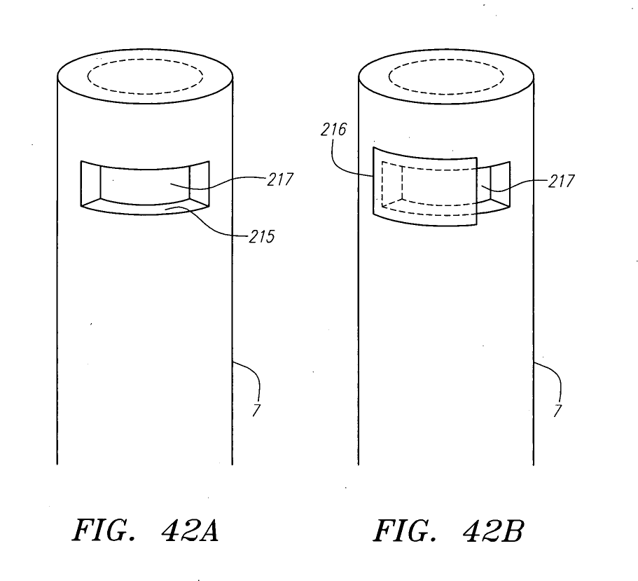

FIG. 42 shows the distal tip of the fluid introduction catheter with a single ejection port for simplicity. This simple construction technique could be used to produce directionality of flow for the ejecting fluid. In FIG. 42A, a notch 215 is made in the catheter 7 creating a port 217. FIG. 42B shows a cover piece 216 placed over a portion of the port 217. This arrangement directs the fluid as it is ejected from the port 217. The cover piece 216 could be attached using heat bonding, adhesives, or other methods common to medical device manufacturing. The port 217 and/or the cover 216 could also be angled to give the desired fluid directionality. This same technique could be used multiple times to produce several such ejection ports 217.

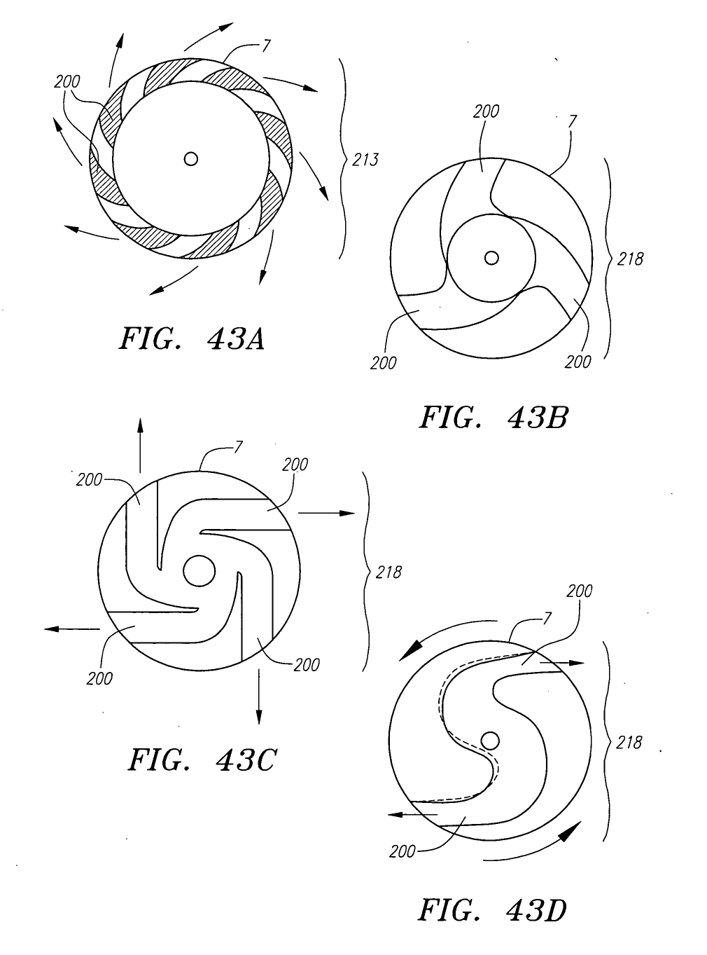

There are many other methods to achieve directionality of flow from the rinse tip. FIG. 43 shows several such techniques. These arrangements can be realized in two distinct manners. One where the distal tip 211 of the catheter 7 is a separate piece 213 that is allowed or caused to rotate by the ejection of fluid through off-center ports. The second has the distal tip 218 secured to the catheter 7 so that the ejection of fluid in a similar off-center or angled manner results in the creation of circular fluid dynamics in the fluid surrounding the catheter 7 but the catheter 7 itself does not rotate. FIG. 43A shows a top view of the distal tip 213 of the catheter 7 with multiple angled ports 200 for the ejection of fluid. FIG. 43B an arrangement 218 with three main ejection ports 200 giving direction to the fluid. FIG. 43C is a top view of the distal tip 218 of the fluid introduction catheter 7 showing four ejection ports 200. By varying the number, size, and arrangement of these ports 200, one can achieve a variety of fluid flow results. FIG. 43D shows a top view of the distal tip 218 of the fluid introduction catheter 7 with two ejection ports 200. With fewer ejection ports 200, the ejection flow can be more regulated and focused.

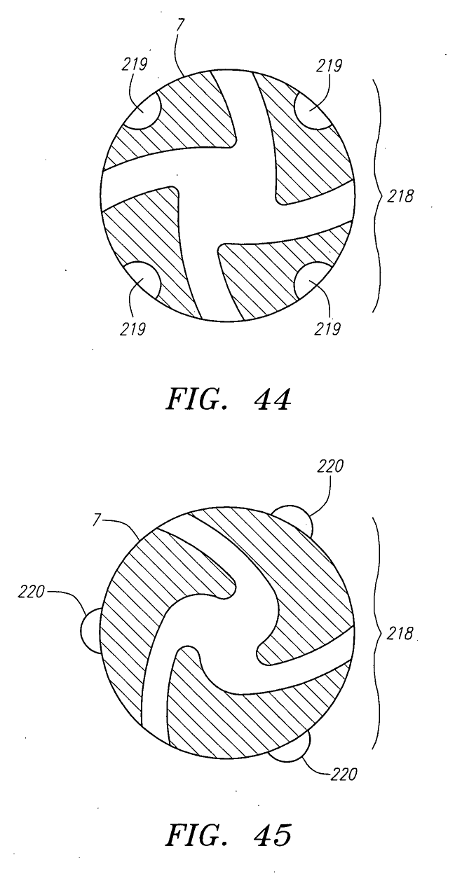

FIG. 44 shows an arrangement of the distal tip 218 of the fluid introduction catheter 7 with the addition of some details of small recessed areas on the exterior of the catheter 7 which serve to engage the surrounding fluid and produce the desired fluid flow response. Likewise, in FIG. 45 similar details are shown, this time protruding to the outside of the catheter 7 but serving a similar purpose of inducing desired flow effects in the surrounding fluid. These details could be added during an additional manufacturing step or could be co-molded with the tip 213.

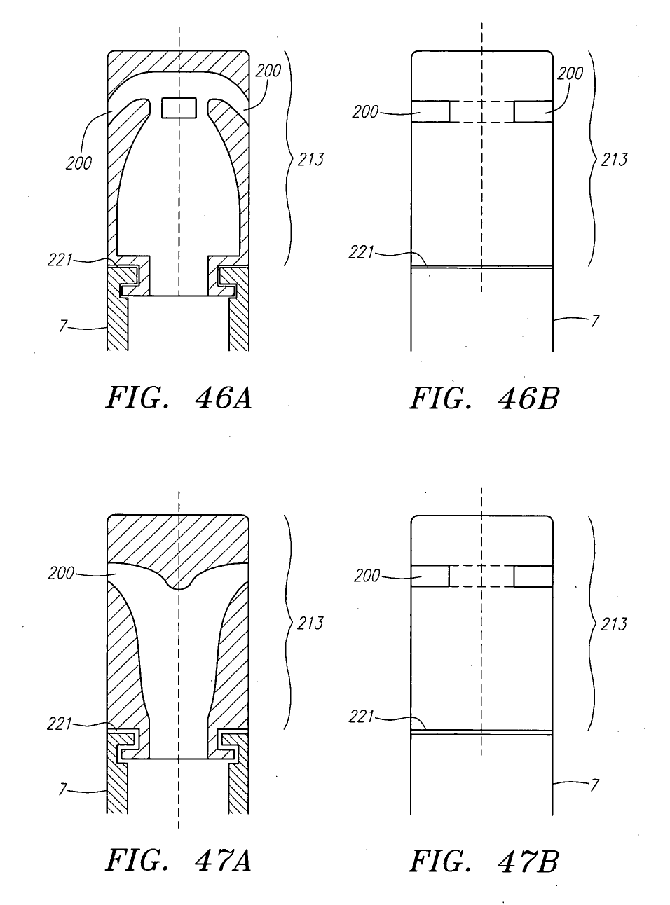

FIG. 46 shows side views of the distal tip of the catheter 7, with a special construction to allow for rotation of the tip 213. FIG. 46A is a cross-sectional drawing showing how the tip 213 could be constructed as two distinct pieces. The juncture 221 between the two would allow for rotation around the longitudinal axis. Such rotation could be driven by the offset ports 200 as discussed previously. FIG. 46B is a side view showing the ports 200 on the exterior of the tip 213. This configuration would allow for fluid to be ejected in the proximal direction and fully circumferentially by the rinse head 213. This would create the desired fluid flows to engage materials that may be partially attached to the walls of the vessel. FIG. 47 shows a similar arrangement with the distinct difference that the rinse tip 213 is constructed such that the fluid would be ejected in the distal direction primarily. This may be of use for the application of certain rinsing agents such as thrombolytic agents, oxygen-rich fluids, plasma, thermal agents as well as many others.

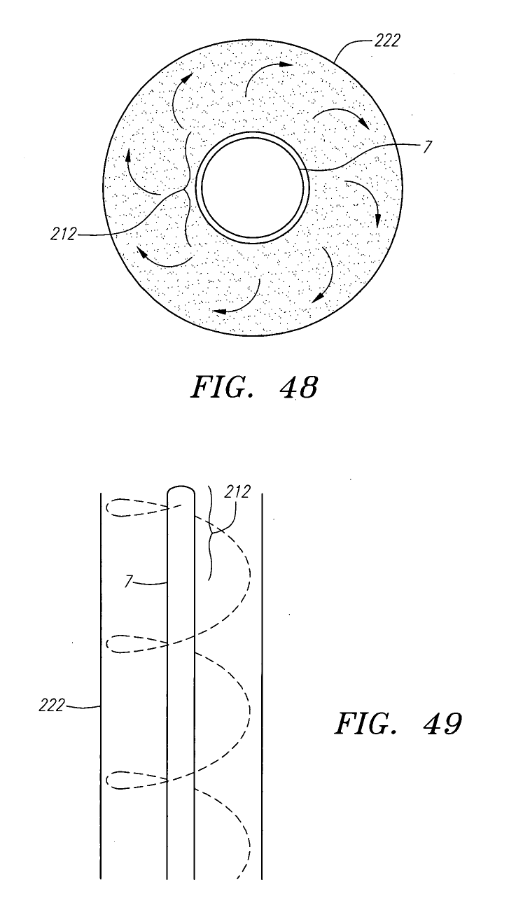

FIG. 48 is a top view of the distal tip of the catheter 212 surrounded by fluid within a vessel 222. A possible fluid flow pattern is indicated by the arrows. This would be the result if the rinse tip of the catheter 222 was held stationary while the fluid was ejected in an offset manner, inducing a circular flow in the surrounding fluid. This shows how such a fluid flow could be advantageous for dislodging emboli or other material that may be up against the vessel walls 222. Since typical laminar flow does not result in as much fluid movement at the walls, this arrangement could be very effective.

FIG. 49 is a side view diagram of the distal tip 212 of the catheter 7 within a vessel 222. The dotted line indicates a potential path of a molecule of the rinsing fluid being introduced. The design of the fluid ejection ports would induce the rotational component as well as send the fluid in the proximal direction. Additionally, the use of a port proximal to the rinse tip would help to create the desired rinse path. This arrangement enhances the contact of the fluid with the vessel wall, optimizing treatment and protection.

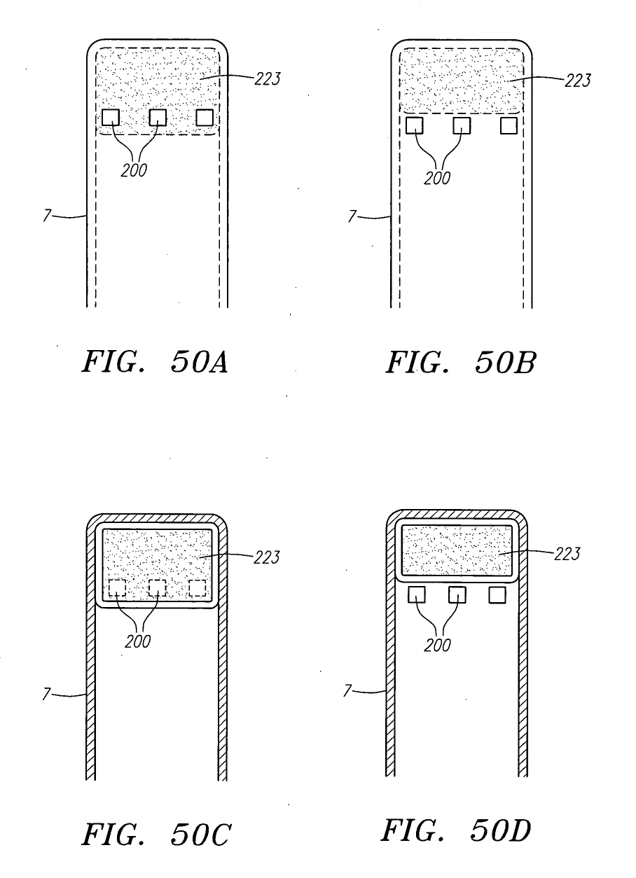

FIG. 50 shows a configuration involving a compressible material located within the distal tip 212 of the catheter 7 allowing for the selective opening and closing of the fluid ports 200. FIG. 50A is a side view showing the compressible material 223 blocking the fluid ports 200. In FIG. 50B, the pressure of the fluid has moved the compressible material so that the ports 200 are revealed and fluid is able to escape. FIGS. 50C and 50D are cross-sectional cut-aways showing similar arrangements. The compressible material 223 could be a sponge like material, coated or not, or an open cell foam piece covered with a thin coating of another material such as silicone or polyurethane.

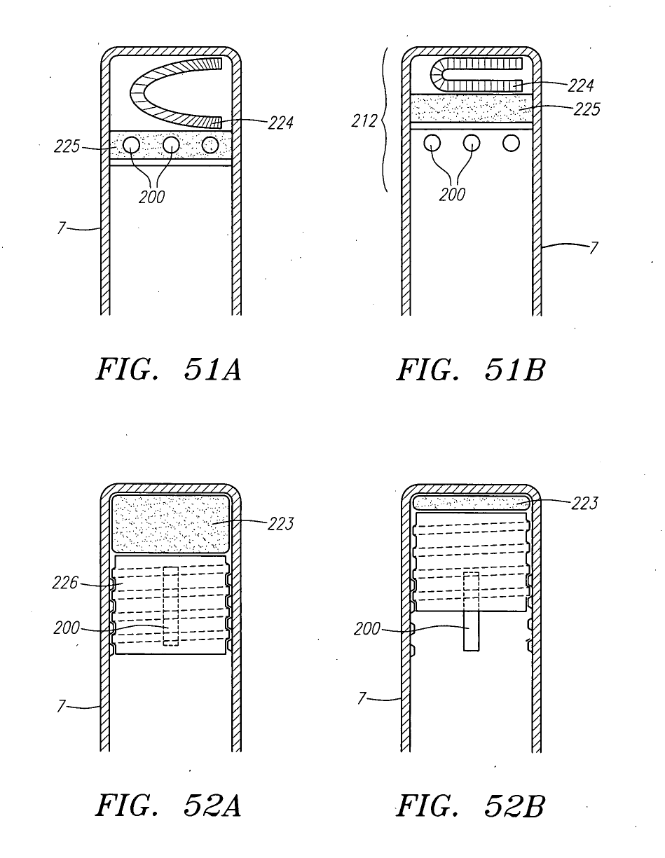

FIG. 51 shows a construction of the distal tip 212 of the fluid introduction catheter 7 designed to offer selective control of the ejection ports 200. FIG. 51A shows a spring 225, in this representation a leaf spring, holding a plug like element 225 over the ejection ports 200. FIG. 51B shows the spring 224 compressed and the plug 225 slid distally to allow fluid to exit the ejection ports 200. The spring 224 could be a leaf spring, simple leaf spring, coiled spring or similar such arrangement. The spring 224 could be made of polymer, metal, memory metal, or similar such material. The spring 224 and plug 225 could be assembled into the tip 212 using heat bonding or adhesive techniques or could be co-molded.

FIG. 52 shows another arrangement of the distal tip 212 of the fluid introduction catheter 7 allowing for the selective opening and closing of the port(s) 200 using a threaded plug 226. FIG. 52A shows the port covered by the plug. FIG. 52B shows the device after the fluid has driven the plug distally and exposed the port 200. A compressible material 223 similar to those discussed earlier is used to return the plug to its default position covering the port.

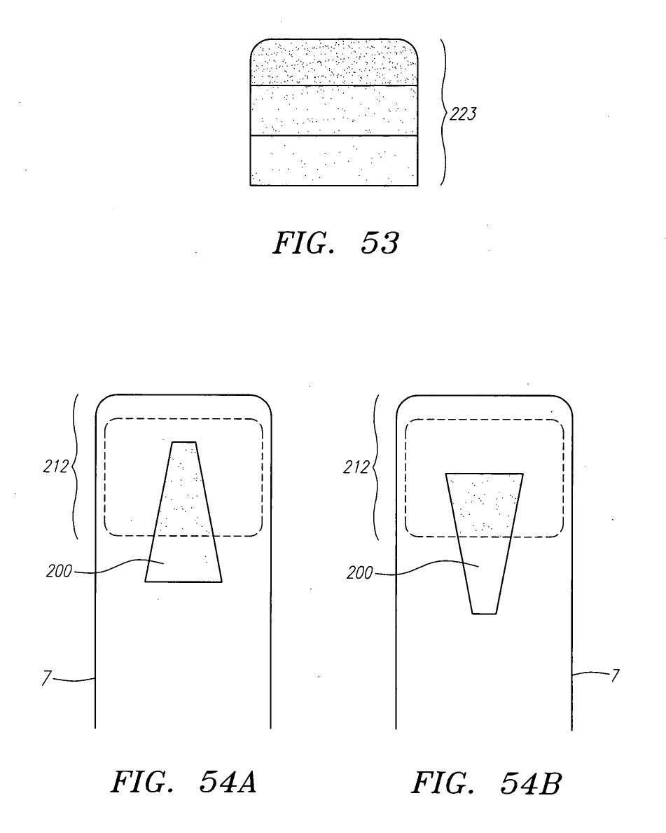

FIG. 53 shows a cross section of a compressible material 223 to indicate that any of the compressible materials referred to herein could be realized with layers of differing materials or densities to yield the desired material response. For example, it may be desired that the material be more resistant to compression as one compresses it more.

FIG. 54 shows side views of the distal tip 212 of the catheter 7 with a varying geometry of the port 200. FIG. 54A has the port 200 getting narrower as it approaches the distal tip 212. This means that as more fluid is introduced, the opening for its ejection is increased at a decreasing rate. FIG. 54B shows the ejection port 200 getting wider toward the distal tip 212. In this arrangement, as more fluid is introduced, the area through which it is ejected increases at an increasing rate. The considerations can be manipulated and combined to achieve the desired flow results.

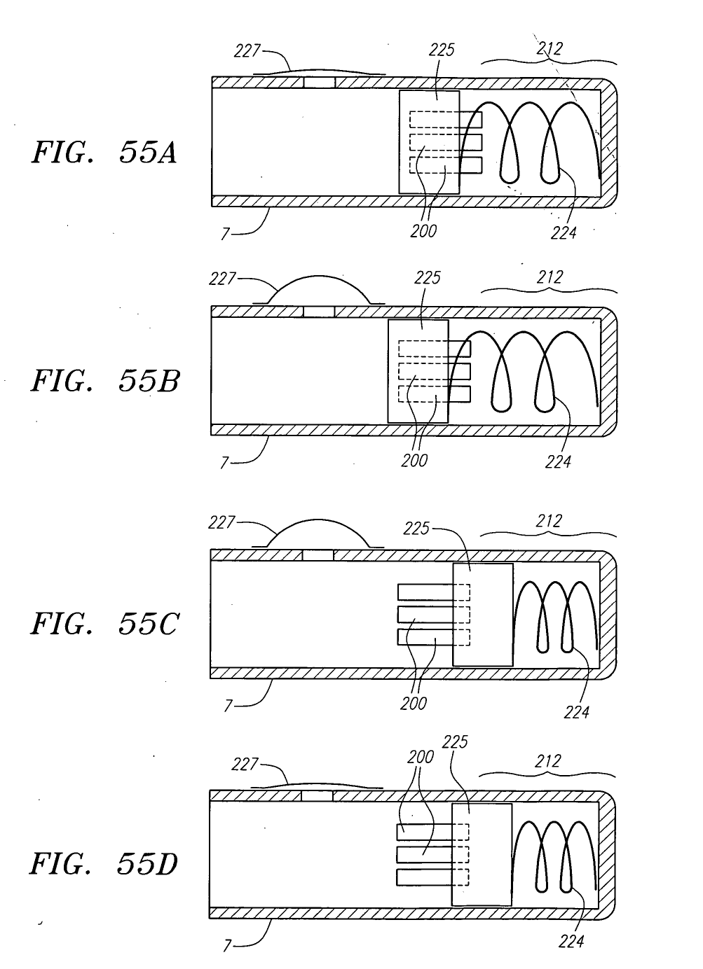

FIG. 55 illustrates a mechanical method for achieving a pulsatile flow at the distal tip 212 of the catheter 7. Essentially, fluid pressure is allowed to build using the side balloon 227. This pressure is then released when the ports 200 are exposed. This is a simple diagram of one method for achieving this concept. FIG. 55A shows the tip system in its default configuration. FIG. 55B shows the side balloon 227 filling with fluid as pressure builds within a lumen of the catheter. Then, when the pressure gets high enough, as in FIG. 55C, the plug 225 within the lumen is slid distal, revealing the ports 200. Fluid would escape through the ports 200 at this time. The pressure would be augmented by the additional fluid stored in the side balloon 227. The side balloon 227 would empty its contents as shown in FIG. 55D. Next, the system would return to its default state of FIG. 55A and the cycle would begin again. The spring 224 behind the plug could be a coil spring, a leaf spring, or simply a compressible material. The side balloon 227 could be manufactured out of materials typical to such an application such as latex, polyurethane, or silicone. The side balloon 227 could be made of a compliant or non-compliant material, depending upon the desired material properties.

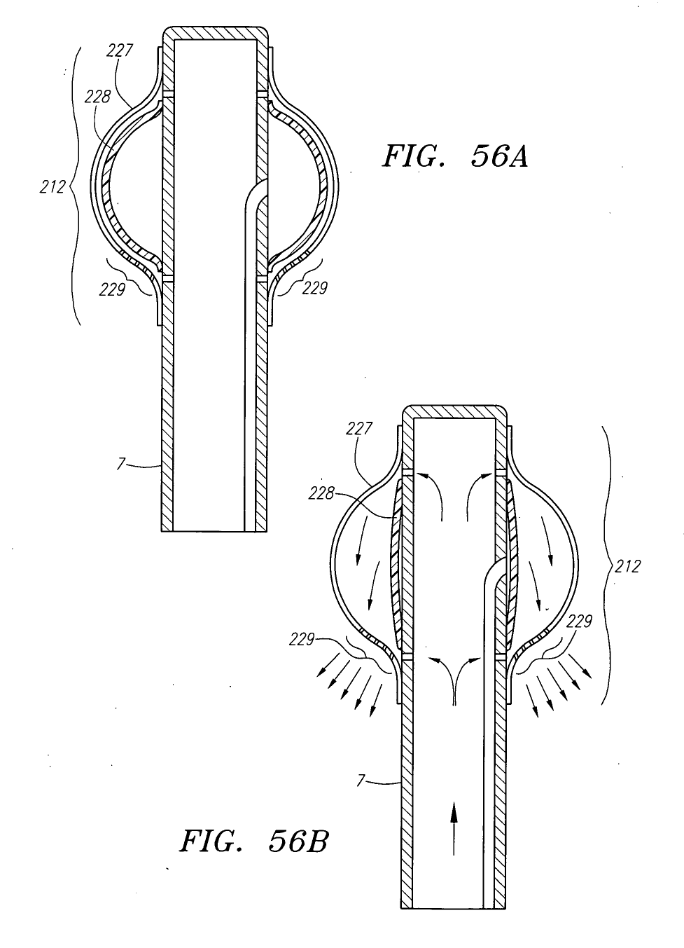

FIG. 56 shows a configuration of the distal tip 212 of the catheter 7 consisting of two balloons, one inside of the other. This allows for one catheter to be able to perform the function of both an angioplasty balloon and the rinse catheter. FIG. 56A shows the embodiment with both of the balloons 227, 229 inflated. This allows the device to be used for angioplasty or other similar procedures. The inner balloon 228 does not allow any of its inflation material to escape. It is filled through the provided inflation lumen. FIG. 56B shows the device with the interior balloon 228 deflated and the exterior balloon 227 full of fluid from the lumen of the catheter 7. Ports 229 in the proximal side of the exterior balloon 227 allow for the introduction of fluid into the vessel. The outer balloon 227 could be made of a compliant material with the inner balloon 228 made of a non-compliant material. This would allow the angioplasty function to be performed effectively, while also allowing the rinsing aspect to not have to occupy an equivalent volume within the vessel. Such a combination of tools would allow the interventionalist to complete the procedure in less time by eliminating an exchange. It could also reduce the cost of the procedure by eliminating a piece of equipment.

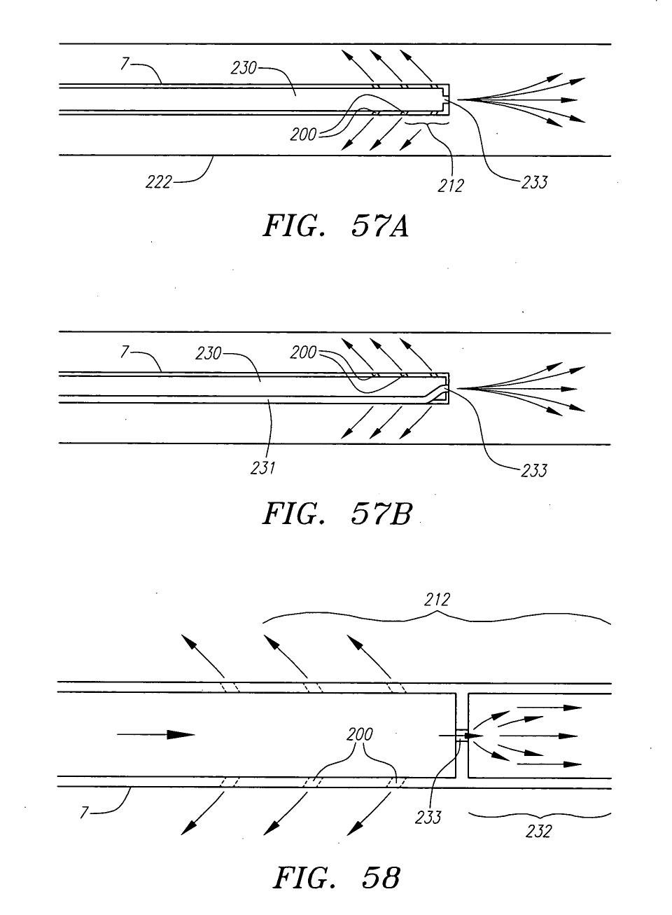

FIG. 57A shows the distal tip 212 of the catheter 7 within a vessel 222. The catheter 7 is a single lumen 230 with ports 200 facing the proximal direction as well as a port 200 to force fluid distally out of the catheter 7. This has several possible advantages. One, this arrangement allows for flow to be maintained distally while also directing fluid and debris or other matter in the proximal direction. If blood, plasma, or an oxygenated blood substitute were utilized as the rinse fluid this would effectively create a liquid shunt. A barrier of stable liquid would be established between the two directions at the tip 212 of the catheter 7. FIG. 57B shows the distal tip 212 of the catheter 7 within a vessel. The catheter 7 is dual lumen. One lumen 230 is dedicated to the fluid that is ejected through the distal port. The other lumen 231 is for the fluid that is ejected through the side ports 200 in a proximal direction. This arrangement allows for complete independence between the two fluids. Entirely different fluids could be introduced and in differing amounts as well. Such fluids include blood, plasma, thermal agents, oxygen-rich fluid, saline, heparinized saline, and thrombolytic agents as well as many others. For example, a blood or oxygen-rich blood substitute or other chemical agent could be sent distally while a saline or other chemical agent could be used to rinse the vessel in the proximal direction: The relative size of the two lumens could be adjusted to achieve the desired flow results.

FIG. 58 is a side view of the distal tip 212 of the fluid introduction catheter 7 detailing a structure extending past the distal port 233 for the purpose of helping to direct and possibly diffuse the fluid as it travels distally. This piece could be formed separately and added or could be co-molded with the catheter 7.

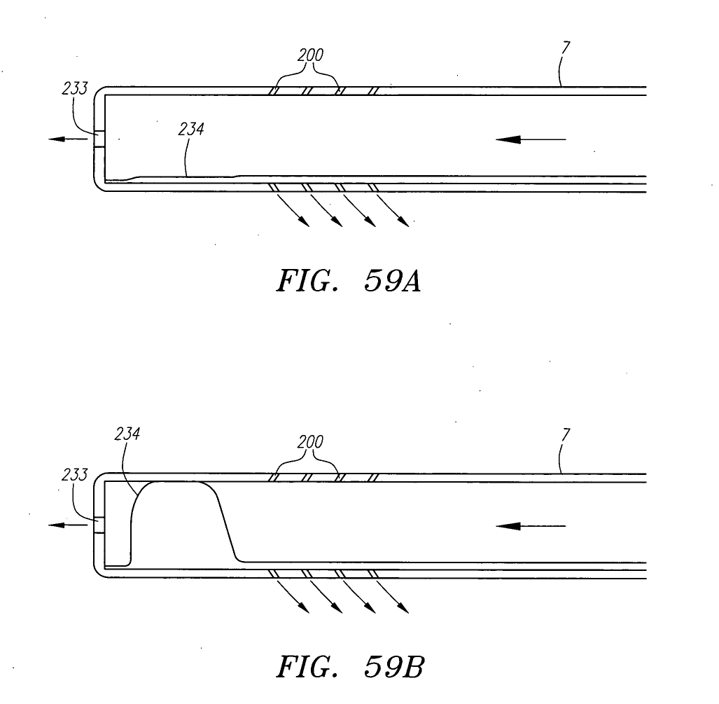

FIG. 59 illustrates a method of selectively opening and closing the distal port 233 of the rinsing catheter using a balloon 234. FIG. 59A shows the balloon 234 deflated and fluid flowing out through the ports 200 in both the distal and the proximal directions. FIG. 59B shows the tip 212 with the interior balloon 234 inflated to block the distal port 233. In this configuration, the fluid would only be able to exit the catheter 7 in the proximal direction. With such an arrangement, the operator could determine when to allow fluid to exit the catheter 7 in the distal direction.

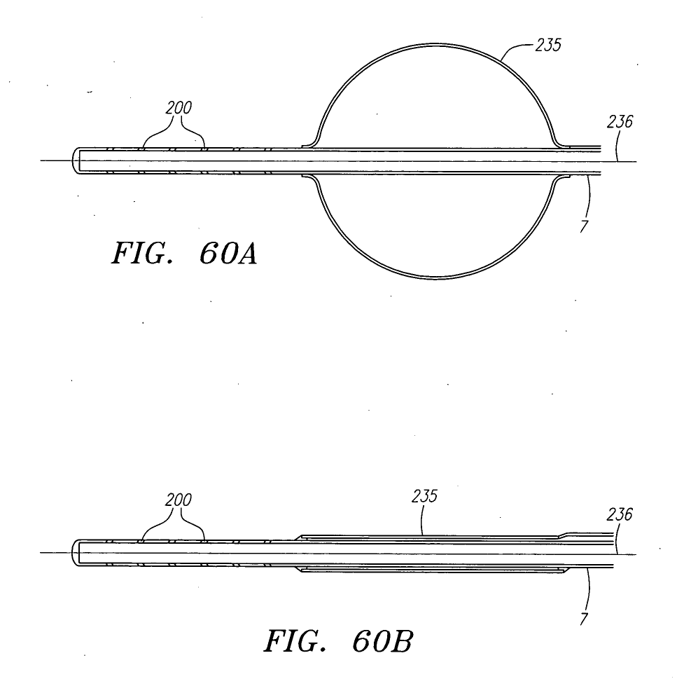

FIG. 60 illustrates another combination of a balloon 234 that could be used for purposes such as angioplasty with a rinsing catheter. FIG. 60A shows the balloon 234 in the inflated formation with the fluid ports 200 located distally to the balloon 234. FIG. 60B shows the balloon 234 deflated and ready for insertion, removal, or rinsing. Such a system reduces the cost and enhances the speed of the operation. Another similar arrangement easy to visualize but not pictured is having some or all of the fluid ports 200 located on the proximal side of the balloon. FIGS. 60A and 60B also show a guidewire running through the center of the rinse catheter.

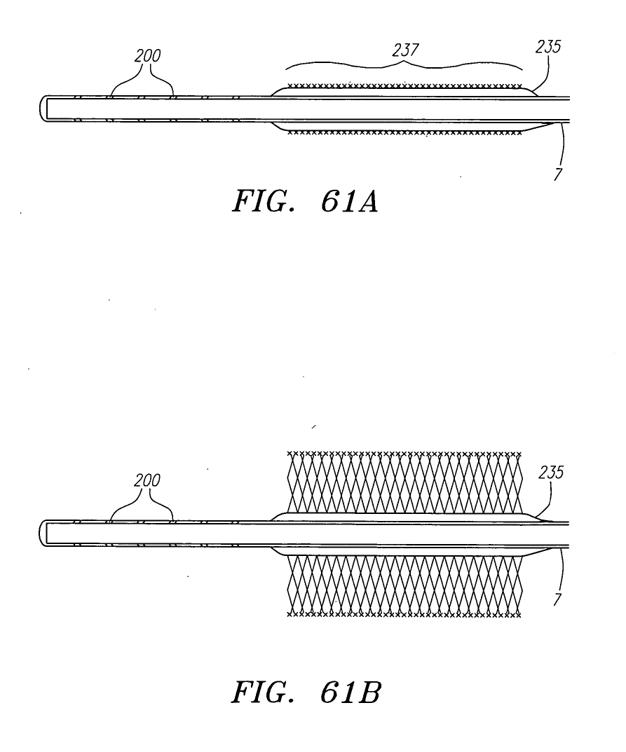

FIG. 61 illustrates one way to combine the rinsing capability with a stent delivery system. FIG. 61A shows the catheter 7 with the stent 237 before it is deployed. FIG. 61B shows the stent 237 deployed and possibly expanded by a balloon 235 as well. The fluid introduction portion of the catheter could be utilized before, during, or after the stenting procedure.

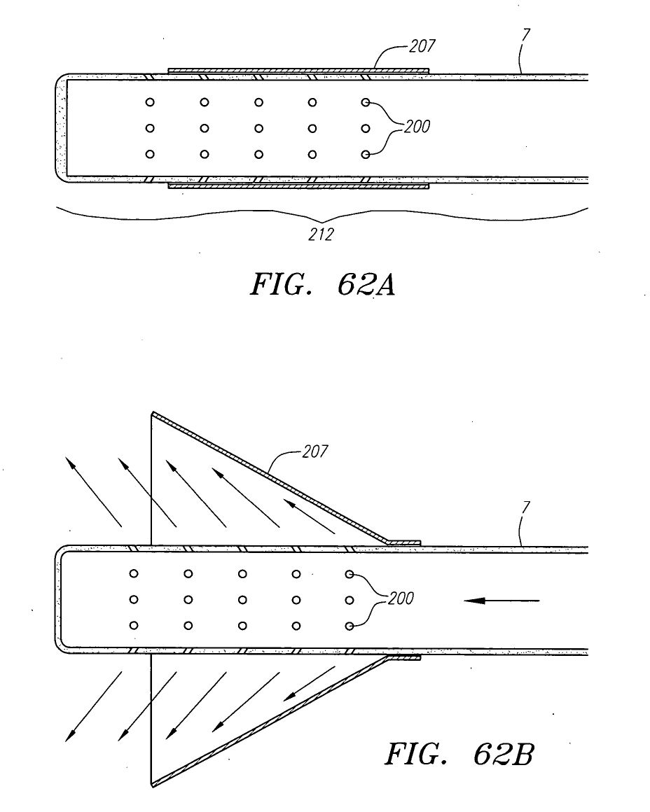

FIGS. 62A and 62B are side views of the distal tip 212 of the catheter 7 showing a configuration to send fluid significantly in the distal direction. FIG. 62A shows the device in its low-profile, insertion state. FIG. 62B shows the device deployed, the cover flap 207 expanded by the pressure of the ejecting fluid, with the cover flap sending fluid largely in the distal direction. Such an arrangement could be used to send a desired fluid distal to the brain, kidneys, heart, or other organ. For instance, an oxygenated fluid, blood, plasma or blood substitute may be desired to be introduced near the brain. The cover flap 207 could be constructed to extend out to the walls of the vessel, thereby isolating the area on its proximal side.

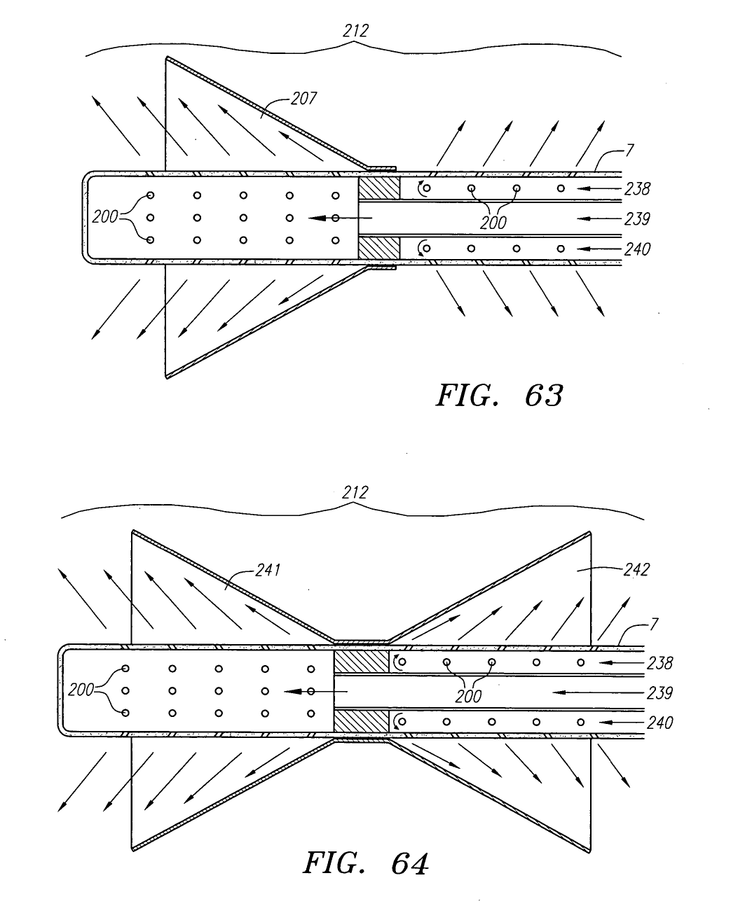

FIG. 63 is a side view showing the distal end 212 of the fluid introduction catheter 7. Here is a construction which allows for the direction of fluid in both the distal and the proximal direction. The multi-lumen construction 238, 239, 240 allows for different fluids to be sent in the two directions. For instance, it may be desired to send an oxygenated fluid, blood, plasma, or blood substitute distal to the brain or other organ while administering diagnostic or therapeutic agents proximal to the catheter tip. The cover flap 207 could be constructed to extend all the way out to the vessel walls to enhance the distinction between the two regions, proximal and distal.

FIG. 64 is a side view showing the distal end 212 of the catheter 7. This arrangement shows two flap structures 207, one for the distal side 241 and one for the proximal side 242. Multiple lumens 238, 239, 240 allow differentiation of the fluids sent proximally and distally.

This arrangement enables the delivery of different fluids in each direction. For instance, it may be desired to send an oxygenated fluid, blood, plasma, or blood substitute distal to the brain or other organ while administering diagnostic or therapeutic agents proximal to the catheter tip. The dual cover flaps 241, 242 work in concert to create a barrier between the proximal and distal regions. In some cases, the cover flaps 241, 242 could be extended to reach the vessel walls, thereby enhancing the barrier between the two regions.

In the recirculation mode, the irrigation and aspiration lumens are in direct fluid communication with the two chambers. Furthermore, the chambers are set to be isolated from fluid communication with either of the reservoirs. The chambers operate in the first half of a cycle with one set of chambers expanding and withdrawing contents from the set of lumens in fluid communication with it causing an aspiration of material from the region near their distal ends, while the other set of chambers shrinks and empties its contents into the other set of lumens causing an infusion of material into the treatment site near the distal end of the catheter 7. In the second half of a cycle, the irrigation chamber shrinks and the aspiration chamber expands, causing the opposite flow pattern as compared to the first half of the cycle. Preferably, the chambers are the same chambers used to produce the action of the first mode. By repeatedly and reversibly activating the plunger 158, the system freely recirculates the selected delivery fluid through the catheter, via lumen 151 and exposes the treatment site at the distal end of the catheter to the irrigation fluid, preferably containing the active pharmaceutical product, without expending additional irrigation fluid that would dilute the activity of the agent.

Thus, by successive, repetitive activations of the system, the constant volume of solution circulates throughout the bypass loop 157, through the lumens 151, and into the treatment site. By setting the two on/off valves 152, 153 and three-way valves 154, 155 to achieve the controlled recirculation, one establishes bi-directional flow through the lumens 151. Upon completion of the desired amount of recirculation, the valves are returned to the configuration appropriate for unidirectional fluid replacement. Activation of the aspiration portion of the system in the conventional configuration described herein then removes the fluid contained through the aspiration side of the system. Clearly, depending on the clinical indication, the foregoing steps can be repeated as often as desired. Alternatively, the control of flow between the chambers, lumens and reservoirs can be performed via one or more multi-port valves. In this instance, the term multi-port valve refers to a valve with a single control (such as a dial or other mechanical control) and four or more ports. The multi-port valves have at least two settings which can be selected by the control, one of which allows for the first mode of operation to take effect, which another of which allows for the second mode of operation to take effect. Each multi-port valve has ports connected to at least one reservoir, at least one chamber and at least one lumen. Each multi-port valve must either incorporate a one-way valve that lies between a chamber and a corresponding lumen when the fluid circuits are by the multi-port valve to be in the first mode of operation, or must have at least one additional port that connects to a one-way valve which lies between a chamber and a corresponding lumen when the fluid circuits are set by the multi-port valve to be in the first mode of operation. Alternatively, a single multi-port valve could be connected via separate ports to at least each of the chambers, each of the reservoirs and at least two sets of lumens. The advantage of the multi-port valves allows for the possibility of simplifying the control of the valves so that the user can set the multi-port valve to a setting corresponding to a desired mode of operation. This would be an improvement over having to ensure that a larger number of valves, each with its own control mechanism, are set properly such they allow the system to operate properly. For example, in transitioning between modes 1 and 2 as described above, the user of a system comprising multi-port valves may only need to change the setting of one or two multi-port valves, rather than have to ensure that several on/off valves and three-way valves are set in a manner that they produce the desired flow circuitry. A further advantage of the use of multi-port valves over a collection of 3-way and on/off valves is a potential reduction in the time required to switch between modes of operation. Optionally, the multi-port valves may allow for a third and/or fourth mode of operation, where an optional third mode of operation would allow for the direct infusion of a fluid into a set of lumens and the optional fourth mode of operation would allow for the direction aspiration of material from a set of lumens.

An example of a method of use of such a system is the following:
1) The therapeutic agent would be introduced into the irrigation chamber of the system.
2) The valve(s) would be set into the arrangement necessary for the system to operate in the fluid exchange mode as described herein.
3) The therapeutic agent would be delivered to the target site with a sufficient number of activations of the irrigation/aspiration system.
4) The valves would then be set into the arrangement necessary to cause recirculation without net fluid replacement.
5) The user would then activate the system for a sufficient number of cycles to achieve the desired therapeutic effect. This is the period in which substantially the same volume of fluid is removed, then reintroduced to produce a mixing effect.
6) The valves would then be returned to the arrangement necessary for them to operate in the fluid exchange mode.
7) Activation of the system could be used to removed the fluid laden with debris resulting from the action of the therapeutic agent, as well as potentially replacing the therapeutic fluid with saline or other rinsing fluid.
8) The system could then be removed.

Many features have been listed with particular configurations, options, and embodiments. Any one or more of the features described may be added to or combined with any of the other embodiments or other standard devices to create alternate combinations and embodiments. Although the examples given include many specificities, they are intended as illustrative of only a few possible embodiments of the invention. Other embodiments and modifications will, no doubt, occur to those skilled in the art. Thus, the examples given should only be interpreted as illustrations of some of the preferred embodiments of the invention.

As noted above, certain fluid flow parameters at the distal end of the catheter are dependent on the relative positioning and geometric arrangement of the infusion and aspiration ports in the distal region of the lumens. Referring to FIG. 20A, in certain indications, the aspiration port(s) 9 may be configured at the distal end of the catheter 7 to remove fluid from a point or points is/are distal to the port(s) 6. Internally, the catheter 7 may be configured such that the irrigation lumen 2 is oriented to be concentrically and annular about the aspiration lumen 3.

Of course, numerous other orientations will be available depending on the desired orientation of the irrigation and aspiration ports. This configuration having aspiration ports distal to irrigation ports 6 has the advantage of tending to promote fluid flow away from a more distally positioned occlusive member (not shown) because the irrigation ports 6 inject fluid at a point more removed from the occluder and the aspiration ports 9 remove fluid from a point or points more immediately adjacent thereto. In this configuration, it is particularly preferred that the catheter element 7 feature a plurality of aspiration ports 9 because this configuration improves fluid flow and turbulence, decreases the possibility that a single port will become clogged with debris, and increases the ability to remove debris at the most distal portion of the catheter 7. This latter attribute is uniquely valuable when the catheter component 7 of the invention is used on the proximal side of an occlusion. The occlusion may be provided by a filter or balloon or may be the result of a pathological condition, such as a total chronic occlusion resulting from disease.

Depending on the nature of the occlusion, in use, the catheter 7 can be advanced to a predetermined point proximal of the occlusion. This is particularly useful in situations such as the “rescue” of a clogged filter or the need to remove debris proximally of an occlusion without actually contacting the occlusion, particularly avoiding direct contact between occlusion and the aspiration ports 9. To achieve this, the catheter 7 may be affixed with a mechanical stop to avoid direct contact between the aspiration ports 9 and the occlusive member, or to fix the distance between the member and the aspiration ports. The mechanical stop prevents excessive suction pressure against the membrane of a filter or balloon in order to reduce the likelihood of rupturing the filtering or occlusive member and prevents the catheter 7 from being advanced too far into the filter.

As noted above, the designation of one lumen as an aspiration lumen 3 and one as an irrigation lumen 2 is essentially functional in nature and the reversal of fluid flow can readily be achieved to take advantage of any clinical situation that warrants altering the conventional irrigation/aspiration orientation. This is particularly true for the above embodiment when used to treat a total chronic occlusion—such as a thrombus. This catheter configuration also takes advantage of the use of an occluding guide having an aspiration lumen 3, to perform a two-stage process for thrombolysis. During a first stage, the aspiration occurs through an aspiration lumen 3 having aspiration ports 9 located at the distal most portion of the catheter 7 to permit intimate contact with the thrombus. Infusion occurs at a more proximate irrigation port 6 or ports in fluid connection with an infusion lumen 2. In this configuration, the extraction of clots and other materials is accomplished more effectively by putting the aspiration lumen 3 near or in direct contact with the material to be extracted. By having the aspiration ports 9 at the very distal tip of the catheter 7, this becomes possible, and effective opening of an occlusion can occur more easily. Once the occlusion is no longer total, the catheter or other devices can be delivered past the point of occlusion. It may then be desirable to switch the conduits used for irrigation and aspiration and perform aspiration using the occluding guide as the aspiration lumen 3, while still using the irrigation lumen 2 of the catheter 7 to deliver fluid to the site. This switching of locale of aspiration can be accomplished with a simple 3-way valve placed between the lumen of the guide catheter, the aspiration lumen 3 of the catheter 7, and the aspiration port 9 of the device that actuates the coordinated inspiration and aspiration (e.g. the fluid exchange device of FIG. 4A). Alternatively, it may be desirable to simultaneously aspirate through both the guide catheter and the aspiration lumen 3 of the catheter while infusing through the irrigation lumen 2.

In a variation of this embodiment, an irrigation lumen 2 may terminate in irrigation ports 6 that face distally rather than radially, to deliver thrombolytics or other fluids in the forward direction towards an occlusion or other target of therapy. The aspiration lumen 3 could then be comprised of the lumen of a second catheter whose distal end is disposed in the region close to the site of infusion, such as an occluding guide catheter.

In yet another variation, the removal of mural thrombi and other material from within blood vessels and body cavities is achieved with an aspiration lumen 3 and associated ports 9 that are steerable towards one side of a vessel wall. The steering capability enables more precise placement of the opening(s) of the aspiration lumens proximate to the material to be removed. As described herein, the aspiration port(s) 9 may face distally, or may face radially, with the specific configuration depending in part on the kind and geometry of the thrombus or other material to be removed. This steering capability is readily provided in the known catheter technology and can be implemented simply by placing a bend in the distal end of the catheter such that the distal tip of the catheter biased to one side, such that the orientation is controllable by simply rotating the catheter containing the aspiration lumen 3. Alternatively, the catheter 7 may incorporate one or more balloons placed asymmetrically around the circumference of the catheter, which, when inflated cause an asymmetric movement of the distal tip of the catheter to the side opposite of the most substantial inflation. Each balloon may be attached such that it does not entirely circumscribe the distal region of the catheter 7, or may be constructed and/or affixed to the catheter such that its expansion causes an asymmetric dilitation of the balloon, relative to the catheter. Alternatively, the catheter may incorporate one or more thin wires that travel substantially within separate lumens of the catheter, whose distal tip is more deformable then the rest of the catheter. By pushing and/or pulling these wires, the distal tip of the catheter can be deflected in a steerable fashion. Alternatively, the catheter may incorporate one or more thin wires that travel substantially within separate lumens of the catheter and are fixed to the distal end of the catheter, but do not travel within the confines of the catheter or any of its lumens for a portion of the distal region of the catheter. By pushing on these wires, they will be forced to buckle in a predictable direction away from the catheter within the distal region and could extend to the vessel wall or cavity wall, thus pushing the catheter towards the opposite wall.

1. A system for fluid exchange within a localized region of the body comprising:
an irrigation reservoir in fluid communication with a chamber and an irrigation lumen, wherein the chamber controls delivery of irrigant fluid from the irrigation reservoir through the irrigation lumen to a target site;
an aspiration lumen having means for controlled collection of aspirant fluid through an aspiration lumen; and
a catheter element comprised of the irrigation lumen terminating in at least one irrigation port, the aspiration lumen terminating in at least one aspiration port, wherein the at least one irrigation and aspiration ports are located at a distal end of the catheter element such that fluid volume exchange occurs between the at least one irrigation port and the at least one aspiration port; and
an occluding element proximal to the at least one irrigation port and the at least one aspiration port, and wherein the catheter lacks a more distal occluding element in the system.

2. The system of claim 1 wherein the aspiration lumen is further comprised of a branch establishing fluid connection with a second aspiration chamber.

3. The system of claim 1 wherein the aspiration lumen is a branched lumen further comprised of a three-way valve.

4. The system of claim 1 wherein the aspiration lumen is a branched lumen further comprised of a one-way valve.

5. The system of claim 1 wherein the aspiration lumen is a branched lumen further comprised of a one-way valve and a three-way valve.

6. The system of claim 5 wherein the aspiration lumen is further comprised of a bypass loop.

7. The system of claim 1 wherein the occluding element is a balloon.

8. The system of claim 1 wherein irrigation lumen is further comprised of a one-way valve.

9. The system of claim 1 wherein the irrigation lumen is further comprised of a three-way valve.

10. The system of claim 1 wherein the irrigation lumen is further comprised of a one-way valve.

11. The system of claim 1 wherein the irrigation lumen is further comprised of a one-way valve and a three-way valve.

12. The system of claim 11 wherein the branched lumen is further comprised of a bypass loop.

13. The system of claim 1 wherein the at least one aspiration port is distal to the at least one irrigation port.

14. The system of claim 13 wherein each at least one aspiration port is located distal to each at least one irrigation port.
